(19) United States
(12) Patent Application Publication
(10) Pub. No.: US 2013/0231373 A1
(43) Pub. Date: Sep. 5, 2013

(54) HYDROXAMATE-BASED INHIBITORS OF DEACETYLASES

(71) Applicants: Clinton Alan Brooks, Beaufort, NC (US); Christine Hiu-Tung Chen, Waltham, MA (US); Young Shin Cho, Cambridge, MA (US); Lei Jiang, Shanghai (CN); Gang Liu, Waltham, MA (US); Michael David Schultz, Lexington, MA (US)

(72) Inventors: Clinton Alan Brooks, Beaufort, NC (US); Christine Hiu-Tung Chen, Waltham, MA (US); Young Shin Cho, Cambridge, MA (US); Lei Jiang, Shanghai (CN); Gang Liu, Waltham, MA (US); Michael David Schultz, Lexington, MA (US)

(73) Assignee: Novartis AG, Basel (CH)

(21) Appl. No.: 13/818,779

(22) PCT Filed: Aug. 27, 2010
(86) PCT No.: PCT/EP10/62594

§ 371 Date: May 8, 2013

(51) Int. Cl. C07D 413/06; C07D 403/06; C07D 401/14

(52) U.S. Cl. CPC C07D 413/06; C07D 401/14; C07D 403/06
USPC 514/343; 435/375; 514/364; 514/406; 514/414; 514/422; 546/268.4; 548/131; 548/255; 548/364.1; 548/455; 548/466; 548/468

(57) ABSTRACT

The present teachings relate to compounds of Formula (I): and pharmaceutically acceptable salts, hydrates, esters, and prodrugs thereof, wherein R¹, R², R³, R⁴, R⁵, ring A, and Z are as defined herein. The present teachings also provide methods of preparing compounds of Formula (I) and methods of use compounds of Formula (I) in treating pathologic conditions or disorders mediated wholly or in part by deacetylases.

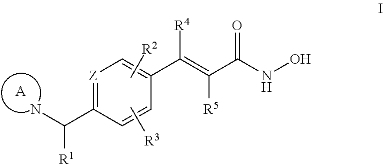

### BACKGROUND OF THE INVENTION

Deacetylation, catalyzed by deacetylases, relates to transcriptional regulation of proteins involved in signal transduction. Accordingly, deacetylase inhibitors can be used for the therapy of pathological conditions or disorders wholly or in part mediated by one or more deacetylases. These conditions or disorders can include retinopathies, age-related macula degeneration, psoriasis, haemangioblastoma, haemangioma, arteriosclerosis, muscle wasting conditions such as muscular dystrophies, cachexia, Huntington's syndrome, inflammatory diseases such as rheumatoid or rheumatic inflammatory diseases, and neoplastic diseases. More specifically, deacetylase inhibitors can be useful for treating arthritis and arthritic conditions (e.g., osteoarthritis, rheumatoid arthritis, and the like), other chronic inflammatory disorders (e.g., chronic asthma, arterial or post-transplantational atherosclerosis, endometriosis, and the like), solid tumors (e.g., cancers of the gastrointestinal tract, pancreas, breast, stomach, cervix, bladder, kidney, prostate, esophagus, ovaries, endometrium, lung, brain, melanoma, Kaposi's sarcoma, squamous cell carcinoma of head and neck, malignant pleural mesotherioma, lymphoma, multiple myeloma, and the like), and liquid tumors (e.g., leukemias).

More specifically, histone deacetylases remove an acetyl group from an N-acetyl lysine on a histone. In normal cells, histone deacetylase (HDAC) and histone acetyltransferase together control the level of acetylation of histones to maintain a balance. Reversible acetylation of histones is a major regulator of gene expression that acts by altering accessibility of transcription factors to DNA.

HDAC inhibitors have been studied for their therapeutic effect to proliferative diseases, including tumors, hyperproliferative conditions, neoplasias, immune diseases, and central and peripheral nervous system diseases. More specifically, HDAC inhibitors can be useful for their antitumor activities. For example, butyric acid and its derivatives, including sodium phenylbutyrate, have been reported to induce apoptosis in vitro in human colon carcinoma, leukemia, and retinoblastoma cell lines. However, butyric acid and its derivatives are not useful as pharmacological agents because they tend to be metabolized rapidly and have a very short half-life in vivo. Other HDAC inhibitors that have been studied for their anti-cancer activities include trichostatin A and trapoxin. Trichostatin A, an antifungal and antibiotic agent, is a reversible inhibitor of mammalian HDAC and trapoxin, a cyclic tetrapeptide, is an irreversible inhibitor of mammalian HDAC. Although trichostatin and trapoxin have been studied for their anti-cancer activities, the in vivo instability of these compounds makes them less suitable as anti-cancer drugs.

### SUMMARY

The present teachings relate to compounds of Formula I:

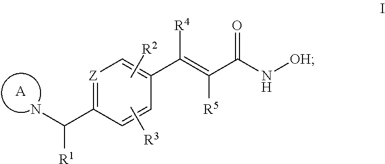

and pharmaceutically acceptable salts, hydrates, esters, and prodrugs thereof, wherein R¹, R², R³, R⁴, R⁵, ring A, and Z are as defined herein.

The present teachings also relate to methods of preparing compounds of Formula I, including pharmaceutically acceptable salts, hydrates, esters and prodrugs thereof, and methods of using compounds of Formula I, including pharmaceutically acceptable salts, hydrates, esters and prodrugs thereof, in treating pathologic conditions or disorders mediated wholly or in part by deacetylases, for example, including administering a therapeutically effective amount of a compound of Formula I to a patient, for example, a patient in need thereof. Examples of the pathologic conditions or disorders include undesired proliferative conditions, neurodegenerative diseases, cardiovascular diseases, strokes, autoimmune diseases, inflammatory diseases, undesired immunological processes, and fungal infections.

The foregoing as well as other features and advantages of the present teachings will be more fully understood from the following description and claims.

### DETAILED DESCRIPTION

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components.

The use of the term “include,” “includes,” “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±5% variation from the nominal value.

It should be understood that the order of steps or order for performing certain actions is immaterial so long as the present teachings remain operable. Moreover, two or more steps or actions may be conducted simultaneously.

As used herein, a “compound” refers to the compound itself and its pharmaceutically acceptable salts, hydrates, and esters, unless otherwise understood from the context of the description or expressly limited to one particular form of the compound, i.e., the compound itself, or a pharmaceutically acceptable salt, hydrate, or ester thereof.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. In some embodiments, an alkyl group can have from 1 to 10 carbon atoms (e.g., from 1 to 6 carbon atoms). Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. In some embodiments, alkyl groups optionally can be substituted with up to four groups independently selected from -L-R⁹ and -L-R¹³, where L, R⁹, and R¹³ are as described herein. A lower alkyl group typically has up to 4 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more carbon-carbon double bonds. In some embodiments, an alkenyl group can have from 2 to 10 carbon atoms (e.g., from 2 to 6 carbon atoms). Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups optionally can be substituted with up to four groups independently selected from -L-R⁹ and -L-R¹³, where L, R⁹, and R¹³ are as described herein.

As used herein, “alkynyl” refers to a straight-chain or branched alkyl group having one or more carbon-carbon triple bonds. In some embodiments, an alkynyl group can have from 2 to 10 carbon atoms (e.g., from 2 to 6 carbon atoms). Examples of alkynyl groups include ethynyl, propynyl, butynyl, pentynyl, and the like. The one or more carbon-carbon triple bonds can be internal (such as in 2-butyne) or terminal (such as in 1-butyne). In some embodiments, alkynyl groups optionally can be substituted with up to four groups independently selected from -L-R⁹ and -L-R¹³, where L, R⁹, and R¹³ are as described herein.

As used herein, “alkoxy” refers to an —O-alkyl group. Examples of alkoxy groups include methoxy, ethoxy, propoxy (e.g., n-propoxy and isopropoxy), t-butoxy groups, and the like.

As used herein, “alkylthio” refers to an —S-alkyl group. Examples of alkylthio groups include methylthio, ethylthio, propylthio (e.g., n-propylthio and isopropylthio), t-butylthio groups, and the like.

As used herein, “haloalkyl” refers to an alkyl group having one or more halogen substituents. In some embodiments, a haloalkyl group can have 1 to 10 carbon atoms (e.g., from 1 to 6 carbon atoms). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.” For example, a C₁₋₁₀ haloalkyl group can have the formula —CᵢH₂ᵢ₊₁₋ⱼXⱼ, wherein X is F, CI, Br, or I, i is an integer in the range of 1 to 10, and j is an integer in the range of 0 to 21, provided that j is less than or equal to 2i+1.

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups optionally can be substituted with up to four groups independently selected from -L-R⁹ and -L-R¹³, where L, R⁹, and R¹³ are as described herein. For example, cycloalkyl groups can be substituted with one or more oxo groups.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group that contains at least one (e.g., one, two, three, four, or five) ring heteroatom selected from O, N, and S, and optionally contains one or more (e.g., one, two, or three) double or triple bonds. A cycloheteroalkyl group, as a whole, can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). In some embodiments, nitrogen atoms of cycloheteroalkyl groups can bear a substituent, for example, a -L-R⁹ or -L-R¹³ group, where L, R⁹, and R¹³ are as described herein. Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, and the like. In some embodiments, cycloheteroalkyl groups optionally can be substituted with up to four groups independently selected from -L-R⁹ and -L-R¹³, where L, R⁹, and R¹³ are as described herein.

As used herein, “aryl” refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms. The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, each aryl group optionally can be substituted with up to four groups independently selected from -L-R⁹ and -L-R¹³, where L, R⁹, and R¹³ are as described herein.

As used herein, “heteroaryl” refers to an aromatic monocyclic ring system containing at least one ring heteroatom selected from O, N, and S or a polycyclic ring system where at least one of the rings in the ring system is aromatic and contains at least one ring heteroatom. A heteroaryl group, as a whole, can have from 5 to 14 ring atoms and contain 1-5 ring heteroatoms. In some embodiments, heteroaryl groups can include monocyclic heteroaryl rings fused to one or more aromatic carbocyclic rings, non-aromatic carbocyclic rings, or non-aromatic cycloheteroalkyl rings. The heteroaryl group can be covalently attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered and 6-membered monocyclic and 5-6 bicyclic ring systems shown below:

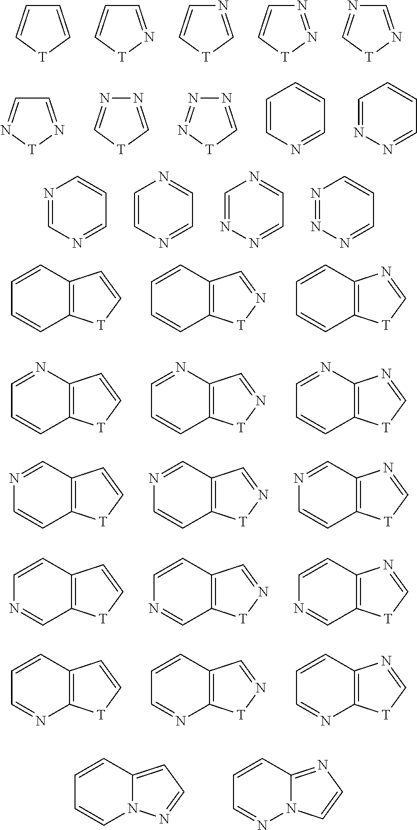

where T is O, S, NH, N-L-R⁹, or N-L-R¹³, where L, R⁹, and R¹³ are as defined herein. Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted with up to four groups independently selected from -L-R⁹ and -L-R¹³, where L, R⁹, and R¹³ are as described herein.

The compounds of the present teachings can include a “divalent group” defined herein as a linking group capable of forming a covalent bond with two other moieties. For example, compounds described herein can include a divalent C₁₋₁₀ alkyl group, such as, for example, a methylene group.

As used herein, a “leaving group” (“LG”) refers to a charged or uncharged atom (or group of atoms) that can be displaced as a stable species as a result of, for example, a substitution or elimination reaction. Examples of leaving groups include, but are not limited to, halide (e.g., Cl, Br, I), azide (N₃), thiocyanate (SCN), nitro (NO₂), cyanate (CN), tosylate (toluenesulfonate, OTs), mesylate (methanesulfonate, OMs), brosylate (p-bromobenzenesulfonate, OBs), nosylate (4-nitrobenzenesulfonate, ONs), water (H₂O), ammonia (NH₃), and triflate (trifluoromethanesulfonate, OTf).

At various places in the present specification, substituents of compounds are disclosed in groups or in ranges. It is specifically intended that the description includes each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 ring atoms; and the phrase “optionally substituted with 1-4 groups” is specifically intended to individually disclose a chemical group that can include 0, 1, 2, 3, 4, 0-4, 0-3, 0-2, 0-1, 1-4, 1-3, 1-2, 2-4, 2-3, and 3-4 groups.

Compounds described herein can contain an asymmetric atom (also referred as a chiral center) and some of the compounds can contain two or more asymmetric atoms or centers, which can thus give rise to optical isomers (enantiomers) and diastereomers (geometric isomers). Compounds of the present teachings include such optical isomers and diastereomers in their respective enantiomerically pure forms (i.e., (+) and (−) stereoisomers), in racemic mixtures, and in other mixtures of the (+) and (−) stereoisomers, as well as pharmaceutically acceptable salts, hydrates, and esters thereof. Optical isomers in pure form or in enantiomerically enriched mixture can be obtained by standard procedures known to those skilled in the art, which include, but are not limited to, chiral separation, diastereomeric salt formation, kinetic resolution, and asymmetric synthesis. The present teachings also encompass cis and trans-isomers of compounds containing alkenyl moieties (e.g., alkenes and imines). It is also understood that the present teachings encompass all possible regioisomers and mixtures thereof, which can be obtained in pure form or in substantially enriched mixture by standard separation procedures known to those skilled in the art, including, but are not limited to, column chromatography, thin-layer chromatography, simulated moving-bed chromatography, and high-performance liquid chromatography.

In one aspect, the present teachings provide compounds of Formula I:

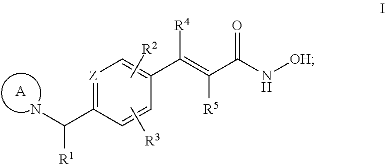

and pharmaceutically acceptable salts, hydrates, esters, and prodrugs thereof,

wherein:

ring A, including the nitrogen atom (N), is a 5 membered cycloheteroalkyl group optionally substituted with 1-4 —Y—R⁶ groups;

Y, at each occurrence, is a) a divalent C₁₋₁₀ alkyl group, b) a divalent C₂₋₁₀ alkenyl group, c) a divalent C₂₋₁₀ alkynyl group, or d) a covalent bond, wherein each of a)-c) optionally is substituted with 1-4 R⁹;

Z is a) CH or b) N;

R¹ is a) H, b) a C₁₋₁₀ alkyl group, c) a C₂₋₁₀ alkenyl group, d) a C₂₋₁₀ alkynyl group, e) a C₃₋₁₄ cycloalkyl group, or f) a 3-14 membered cycloheteroalkyl group, wherein each of b)-f) optionally is substituted with 1-4 -L-R⁹ groups;

R², R³, R⁴, and R⁵ independently are a) H or b) halogen;

R⁶, at each occurrence, is a) H, b) halogen, c) —OR′, d) —NR⁷R⁸, e) a C₁₋₁₀ alkyl group, f) a C₂₋₁₀ alkenyl group, g) a C₂₋₁₀ alkynyl group, h) a C₃₋₁₄ cycloalkyl group, i) a C₆₋₁₄ aryl group, j) a 3-14 membered cycloheteroalkyl group, or k) a 5-14 membered heteroaryl group, wherein each of e)-k) optionally is substituted with 1-4 -L-R⁹ groups, or

two —Y—R⁶ groups, taken together with the atom to which each —Y—R⁶ group is attached and any intervening ring atoms, form a) a C₃₋₁₄ cycloalkyl group or b) a 3-14 membered cycloheteroalkyl group, wherein each of a)-b) optionally is substituted with 1-4 R⁹ groups;

R⁷ and R⁸, at each occurrence, independently are a) H, b) —C(O)R¹¹, c) —S(O)ₘR¹¹, d) a C₁₋₁₀ alkyl group, e) a C₂₋₁₀ alkenyl group, f) a C₂₋₁₀ alkynyl group, g) a C₃₋₁₄ cycloalkyl group, h) a C₆₋₁₄ aryl group, i) a 3-14 membered cycloheteroalkyl group, or j) a 5-14 membered heteroaryl group, wherein each of d)-j) optionally is substituted with 1-4 -L-R⁹ groups;

R⁹, at each occurrence, is a) halogen, b) —CN, c) —NO₂, d) oxo, e) ═N-L-R¹⁰, f) —O-L-R¹⁰, g) —NR¹⁰-L-R¹⁰, h) a C₁₋₁₀ alkyl group, i) a C₁₋₁₀ haloalkyl group, j) a C₂₋₁₀ alkenyl group, k) a C₂₋₁₀ alkynyl group, l) a C₃₋₁₄ cycloalkyl group, m) a C₆₋₁₄ aryl group, n) a 3-14 membered cycloheteroalkyl group, or o) a 5-14 membered heteroaryl group, wherein each of h)-o) optionally is substituted with 1-4 -L-R¹³ groups;

R¹⁰, at each occurrence, is a) H, b) —OR¹¹, c) —NR¹¹R¹², d) —C(O)R¹¹, e) —S(O)ₘR¹¹, f) a C₁₋₁₀ alkyl group, g) a C₂₋₁₀ alkenyl group, h) a C₂₋₁₀ alkynyl group, i) a C₃₋₁₄ cycloalkyl group, j) a C₆₋₁₄ aryl group, k) a 3-14 membered cycloheteroalkyl group, or 1) a 5-14 membered heteroaryl group, wherein each of f)-l) optionally is substituted with 1-4 -L-R¹³ groups;

R¹¹ and R¹², at each occurrence, independently are a) H, b) a C₁₋₄₀ alkyl group, c) a C₂₋₁₀ alkenyl group, d) a C₂₋₁₀ alkynyl group, e) a C₃₋₁₄ cycloalkyl group, f) a C₆₋₁₄ aryl group, g) a 3-14 membered cycloheteroalkyl group, or h) a 5-14 membered heteroaryl group, wherein each of b)-h) optionally is substituted with 1-4 -L-R¹³ groups;

R¹³, at each occurrence, is a) halogen, b) —CN, c) —NO₂, d) oxo, e) —OH, f) —NH₂, g) —NH(C₁₋₁₀ alkyl), h) —N(C₁₋₁₀ alkyl)₂, i) —CHO, j) —C(O)—C₁₋₁₀ alkyl, k) —C(O)OH,

l) —C(O)—O(C₁₋₁₀ alkyl), m) —C(O)SH, n) —C(O)—SC₁₋₁₀ alkyl, o) —C(O)NH₂, p) —C(O)NH(C₁₋₁₀ alkyl), q) —C(O)N(C₁₋₁₀ alkyl)₂, r) —C(S)H, s) —C(S)—C₁₋₁₀ alkyl, t) —C(S)NH₂, u) —C(S)NH(C₁₋₁₀ alkyl), v) —C(S)N(C₁₋₁₀ alkyl)₂, w) —C(NH)H, x) —C(NH)(C₁₋₁₀ alkyl), y) —C(NH)NH₂, z) —C(NH)NH(C₁₋₁₀ alkyl), aa) —C(NH)N(C₁₋₁₀ alkyl)₂, ab) —C(NC₁₋₁₀ alkyl)H, ac) —C(NC₁₋₁₀ alkyl)-C₁₋₁₀ alkyl, ad) —C(NC₁₋₁₀ alkyl)NH(C₁₋₁₀ alkyl), ae) —C(NC₁₋₁₀ alkyl)N(C_{1 -10} alkyl)₂, af) —S(O)ₘH, ag) —S(O)ₘ—Cl₁₋₁₀ alkyl, ah) —S(O)₂OH, ai) —S(O)ₘ—OC₁₋₁₀ alkyl, aj) —S(O)ₘNH₂, ak) —S(O)ₘNH(C₁₋₁₀ alkyl), al) —S(O)ₘ—N(C₁₋₁₀ alkyl)₂, am) —Si(C₁₋₁₀ alkyl)₃, an) a C₁₋₁₀ alkyl group, ao) a C₂₋₁₀ alkenyl group, ap) a C₂₋₁₀ alkynyl group, aq) a C₁₋₁₀ alkoxy group, ar) a C₁₋₁₀ haloalkyl group, as) a C₃₋₁₄ cycloalkyl group, at) a C₆₋₁₄ aryl group, au) a 3-14 membered cycloheteroalkyl group, or av) a 5-14 membered heteroaryl group;

L, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, e) a divalent C₁₋₁₀ alkoxy group, or f) a covalent bond; and

m, at each occurrence, is 0, 1, or 2.

In various embodiments, two —Y—R⁶ groups, taken together with the atom to which each —Y—R⁶ group is attached and any intervening ring atoms, can form a C₃₋₁₄ cycloalkyl group or a 5-14 membered cycloheteroalkyl group, each of which optionally can be substituted with 1-4 R⁹ groups, where R⁹ is as defined herein. In some embodiments, the two —Y—R⁶ groups, taken together with the atom to which each —Y—R⁶ group is attached and any intervening ring atoms, can form a C₃₋₁₄ cycloalkyl group optionally substituted with 1-4 R⁹ groups, where R⁹ is as defined herein. For example, the C₃₋₁₄ cycloalkyl group can be a cyclopentyl group, a cyclohexyl group, or a cycloheptyl group. In certain embodiments, the C₃₋₁₄ cycloalkyl group, taken together with ring A, can be an octahydrocyclopenta[b]pyrrolyl group or an octahydroindoly group, each of which optionally can be substituted with 1-4 R⁹ groups, where R⁹ is as defined herein. In particular embodiments, ring A, taken together with the two —Y—R⁶ groups and optionally substituted with additional 1 or 2 —Y—R⁶ groups, can form an octahydrocyclopenta[b]pyrrolyl group optionally substituted with 1-4 R⁹ groups, where R⁹ is as defined herein.

In various embodiments, compounds of the present teachings can have Formula II:

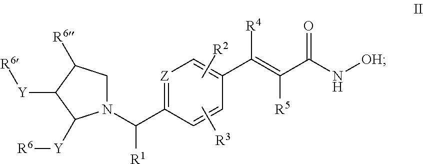

including pharmaceutically acceptable salts, hydrates, esters, and prodrugs thereof,

wherein:

R^{6′} and R^{6″} independently are a) H, b) halogen, c) —OR⁷, d) —NR⁷R⁸, e) a C₁₋₁₀ alkyl group, f) a C₂₋₁₀ alkenyl group, g) a C₂₋₁₀ alkynyl group, h) a C₃₋₁₄ cycloalkyl group, i) a C₆₋₁₄ aryl group, j) a 3-14 membered cycloheteroalkyl group, or k) a 5-14 membered heteroaryl group, wherein each of e)-k) optionally is substituted with 1-4 -L-R⁹ groups; and

R¹, R², R³, R⁴, R⁵, R⁶, R⁷, R⁸, R⁹, L, Y, and Z are as defined herein.

In some embodiments, Y, at each occurrence, can be a covalent bond. In some embodiments, Y, at each occurrence, can be a divalent C₁₋₁₀ alkyl group, a divalent C₁₋₈ alkyl group, a divalent C₁₋₅ alkyl group, or a divalent C₁₋₃ alkyl group, each of which optionally can be substituted with 1-4 R⁹ groups, where R⁹ is as defined herein. In some embodiments, Y, at each occurrence, can be a divalent C₁₋₃ alkyl group optionally substituted with 1-4 R⁹ groups, where R⁹ is as defined herein. In certain embodiments, Y can be selected from —CH₂—, —CH(OH)—, and —C(O)—.

In various embodiments, R⁶ and R^{6′} independently can be selected from H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, and a 5-14 membered heteroaryl group, where each of the C₁₋₁₀ alkyl groups, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally can be substituted with 1-4 -L-R⁹ groups, and L and R⁹ are as defined herein. For example, R⁶ and R^{6′} independently can be selected from H,

a C₁₋₁₀ alkyl group, a C₃₋₁₄ cycloalkyl group, a C₆₋₁₄ aryl group, a 3-14 membered cycloheteroalkyl group, and a 5-14 membered heteroaryl group, where each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group optionally can be substituted with 1-4 -L-R⁹ groups, where L and R⁹ are as defined herein. In some embodiments, R⁶ and R^{6′} independently can be H. In some embodiments, R⁶ can be a C₁₋₁₀ alkyl group optionally substituted with 1-4 -L-R⁹ groups, where L and R⁹ are as defined herein. For example, R⁶ and R^{6′} independently can be a methyl group, an ethyl group, a propyl group, a butyl group, or a hexyl group each optionally substituted with 1-4 -L-R⁹ groups, wherein L and R⁹ are as defined herein. In certain embodiments, R⁶ and R^{6′} independently can be a propyl group. In particular embodiments, R⁶ can be a propyl group.

In some embodiments, R⁶ and R^{6′} independently can be selected from a C₆₋₁₄ aryl group, a 3-14 membered cycloheteroalkyl group, and a 5-14 heteroaryl group, each of which optionally can be substituted with 1-4 -L-R⁹ groups, where L and R⁹ are as defined herein. In certain embodiments, R⁶ and R^{6′} independently can be a C₆₋₁₄ aryl group optionally substituted with 1-4 -L-R⁹ groups, where L and R⁹ are as defined herein. For example, R⁶ and R^{6′} independently can be a phenyl group optionally substituted with 1-4 -L-R⁹ groups, where L and R⁹ are as defined herein. In certain embodiments, R⁶ and R^{6′} independently can be a 3-14 membered cycloheteroalkyl group optionally substituted with 1-4 -L-R⁹ groups, where L and R⁹ are as defined herein. For example, R⁶ and R^{6′} independently can be a pyrrolidinyl group, a tetrahydrofuranyl group, a tetrahydrothiophenyl group, a piperidinyl group, a morpholinyl group, a piperazinyl group, or a hexahydropyrimidinyl group, each of which optionally can be fused to a C₆₋₁₄ aryl group or a 5-14 membered heteroaryl group and optionally can be substituted with 1-4 -L-R⁹ groups, where L and R⁹ are as defined herein. In particular embodiments, R⁶ and R^{6′} independently can be a pyrrolidinyl group or an indolinyl group optionally substituted with 1-4 -L-R⁹ groups, where L and R⁹ are as defined herein. In certain embodiments, R⁶ and R^{6′} independently can be a 5-14 membered heteroaryl group optionally substituted with 1-4 -L-R⁹ groups, where L and R⁹ are as defined herein. For example, R⁶ and R^{6′} independently can be a pyrrolyl group, a pyrazolyl group, a triazolyl group, a furanyl group, an oxazolyl group, an oxadiazolyl group, a thiophenyl group, a thiazolyl group, a thiadiazolyl group, or a tetrazolyl group, each of which optionally can be substituted with 1-4 -L-R⁹ groups, where L and R⁹ are as defined herein. In particular embodiments, R⁶ and R^{6′} independently can be selected from a pyrrolyl group, a pyrazolyl group, a triazolyl group, an oxadiazolyl group, a pyridyl group, an indolyl group, and an indazolyl group, each of which optionally can be substituted with 1-4 -L-R⁹ groups, where L and R⁹ are as defined herein.

In various embodiments, R⁶ and R^{6′} independently can be substituted with 1-4 -L-R⁹ groups, where R⁹ can be selected from halogen, —OH, —O—(C₁₋₁₀ alkyl), —O—(C₃₋₁₄ cycloalkyl), —O—C₆₋₁₄ aryl, —NH₂, —NH(C₁₋₁₀ alkyl), —N(C₁₋₁₀ alkyl)₂, a C₁₋₁₀ alkyl group, a C₁₋₁₀ haloalkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, and a 5-14 membered heteroaryl group, where each of the C₁₋₁₀ alkyl groups, the C₁₋₁₀ haloalkyl group, the C₂₋₁₀ alkenyl group, the C₂₋₁₀ alkynyl group, the C₃₋₁₄ cycloalkyl groups, the C₆₋₁₄ aryl group, the 3-14 membered cycloheteroalkyl group, and the 5-14 membered heteroaryl group optionally can be substituted with 1-4 -L-R¹³ groups, and L and R¹³ are as defined herein. For example, R⁹ can be selected from —OH, —O(C₁₋₁₀ alkyl), a C₁₋₁₀ alkyl group, a C₁₋₁₀ haloalkyl group, a C₃₋₁₄ cycloalkyl group, a C₆₋₁₄ aryl group, and a 5-14 membered heteroaryl group, wherein each of the C₁₋₁₀ alkyl groups, the C₁₋₁₀ haloalkyl group, the C₃₋₁₄ cycloalkyl group, the C₆₋₁₄ aryl group, and the 5-14 membered heteroaryl group optionally is substituted with 1-3 R¹³ groups, where R¹³ is as defined herein. In some embodiments, R⁶ and R^{6′} independently can be substituted with 1-4 groups independently selected from —(C₁₋₁₀ alkyl)-OH, —(C₁₋₁₀ alkyl)-(C₃₋₁₄ cycloalkyl), —(C₁₋₁₀ alkyl)-(C₆₋₁₄ aryl), —(C₁₋₁₀ alkyl)-(3-14 membered cycloheteroalkyl), —(C₁₋₁₀ alkyl)-(5-14 membered heteroaryl), a C₁₋₁₀alkyl group, a C₁₋₁₀ alkoxy group, a C₁₋₁₀ haloalkyl group, a C₆₋₁₄ aryl group, and a 5-14 membered heteroaryl group, each of the C₁₋₁₀ alkyl groups, the C₃₋₁₄ cycloalkyl groups, the C₆₋₁₄ aryl groups, the 3-14 membered cycloheteroalkyl group, and the 5-14 membered heteroaryl groups optionally can be substituted with 1-4 -L-R³ groups, where L and R¹³ are as defined herein. In certain embodiments, R⁶ and R^{6′} independently can be substituted with 1-4 groups independently selected from —CF₃, a methyl group, an ethyl group, an isopropyl group, a tert-butyl group, a cyclohexylmethyl group, a hydroxymethyl group, a 1-hydroxy-1-methylethyl group, a benzyl group, a phenyl group, and a pyridyl group.

In various embodiments, R^{6″} can be H, halogen, —OR⁷, or —NR⁷R⁸, where R⁷ and R⁸ are as defined herein. In some embodiments, R^{6″} can be H, F, Cl, Br, —OH, —O—C₁₋₁₀ alkyl, —NH₂, —NH(C₁₋₁₀ alkyl), or —N(C₁₋₁₀ alkyl)₂, where each of the C₁₋₁₀ alkyl groups optionally can be substituted with 1-4 -L-R¹³ groups, and L and R¹³ are as defined herein. In certain embodiments, R^{6″} can be H, F, —OH, —O(C₁₋₁₀ alkyl), or —NH₂. In particular embodiments, R⁶″ can be H, F, —OH, —OCH₃, or —NH₂.

In various embodiments, compounds of the present teachings can have Formula IIa or Formula IIb:

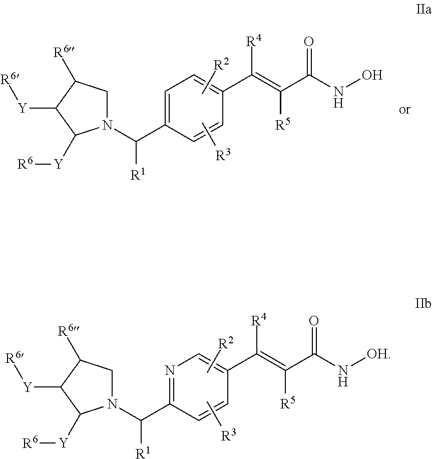

including pharmaceutically acceptable salts, hydrates, esters, and prodrugs thereof, where R¹, R², R³, R⁴, R⁵, R⁶, R^{6′}, R^{6″}, and Y are as defined herein.

In various embodiments, R⁴ can be selected from H, F, Cl, and Br. For example, R⁴ can be H. In various embodiments, R⁵ can be selected from H, F, Cl, and Br. For example, R⁵ can be H or F. In various embodiments R⁴ and R⁵ are both H.

In various embodiments, R² and R³ independently can be selected from H, F, Cl, and Br. In some embodiments, R² can be selected from H or F. In some embodiments, R³ can be selected from H or F. In some embodiments R² and R³ are both H.

In various embodiments, R¹ can be H, a C₁₋₁₀ alkyl group, a C₃₋₁₄ cycloalkyl group, or a 3-14 membered cycloheteroalkyl group, where each of the C₁₋₁₀ alkyl groups, the C₃₋₁₄ cycloalkyl group, and the 3-14 membered cycloheteroalkyl group optionally can be substituted with 1-4 -L-R⁹ groups, and L and R⁹ are as defined herein. For example, R¹ can be H or a C₁₋₁₀ alkyl group optionally substituted with 1-4 -L-R⁹ groups, where L and R⁹ are as defined herein. In some embodiments, R¹ can be H. In some embodiments, R¹ can be a methyl group, an ethyl group, an isopropyl group, a t-butyl group, a cyclopropyl group, a cyclopentyl group, or a cyclohexyl group, each optionally substituted with 1-4 groups independently selected from halogen. In certain embodiments, R¹ can be a methyl group.

In various embodiments compounds of the present teachings can have Formula IIIa or Formula IIIb:

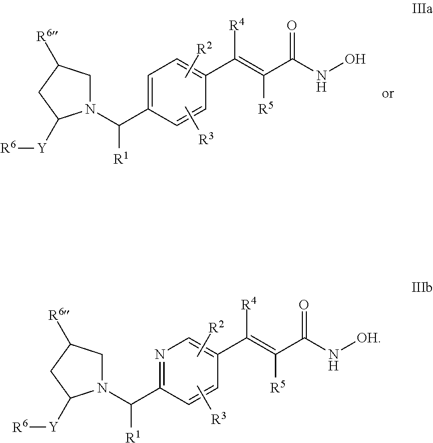

including pharmaceutically acceptable salts, hydrates, esters, and prodrugs thereof, where

R¹ is H or methyl;

R², R³, R⁴, and R⁵ independently are a) H or b) halogen;

Y is a) a divalent C₁₋₁₀ alkyl group, b) a divalent C₂₋₁₀ alkenyl group, c) a divalent C₂₋₁₀ alkynyl group, or d) a covalent bond, wherein each of a)-c) optionally is substituted with 1-4 R⁹;

R⁶ is a) H, b) halogen, c) —OR⁷, d) —NR⁷R⁸, e) a C₁₋₁₀ alkyl group f) a C₂₋₁₀ alkenyl group, g) a C₂₋₁₀ alkynyl group, h) a C₃₋₁₄ cycloalkyl group, i) a C₆₋₁₄ aryl group, j) a 3-14 membered cycloheteroalkyl group, or k) a 5-14 membered heteroaryl group, wherein each of e)-k) optionally is substituted with 1-4 -L-R⁹ groups;
R^{6″} is H, hydroxy, methoxy, NH₂, or fluoro;

R⁷ and R⁸, at each occurrence, independently are a) H, b) —C(O)R¹¹, c) —S(O)ₘR¹¹, d) a C₁₋₁₀ alkyl group, e) a C₂₋₁₀ alkenyl group, f) a C₂₋₁₀ alkynyl group, g) a C₃₋₁₄ cycloalkyl group, h) a C₆₋₁₄ aryl group, i) a 3-14 membered cycloheteroalkyl group, or j) a 5-14 membered heteroaryl group, wherein each of d)-j) optionally is substituted with 1-4 -L-R⁹ groups;

R⁹, at each occurrence, is a) halogen, b) —CN, c) —NO₂, d) oxo, e) ═N-L-R¹⁰, f) —O-L-R¹⁰, g) —NR¹⁰-L-R¹⁰, h) a C₁₋₁₀ alkyl group, i) a C₁₋₁₀ haloalkyl group, j) a C₂₋₁₀ alkenyl group, k) a C₂₋₁₀ alkynyl group, 1) a C₃₋₁₄ cycloalkyl group, m) a C₆₋₁₄ aryl group, n) a 3-14 membered cycloheteroalkyl group, or o) a 5-14 membered heteroaryl group, wherein each of h)-o) optionally is substituted with 1-4 -L-R¹³ groups;

R¹⁰, at each occurrence, is a) H, b) —OR¹¹, c) —NR¹¹R¹², d) —C(O)R¹¹, e) —S(O)ₘR¹¹, f) a C₁₋₁₀ alkyl group, g) a C₂₋₁₀ alkenyl group, h) a C₂₋₁₀ alkynyl group, i) a C₃₋₁₄ cycloalkyl group, j) a C₆₋₁₄ aryl group, k) a 3-14 membered cycloheteroalkyl group, or 1) a 5-14 membered heteroaryl group, wherein each of f)-l) optionally is substituted with 1-4 -L-R¹³ groups;

R¹¹ and R¹², at each occurrence, independently are a) H, b) a C₁₋₁₀ alkyl group, c) a C₂₋₁₀ alkenyl group, d) a C₂₋₁₀ alkynyl group, e) a C₃₋₁₄ cycloalkyl group, f) a C₆₋₁₄ aryl group, g) a 3-14 membered cycloheteroalkyl group, or h) a 5-14 membered heteroaryl group, wherein each of b)-h) optionally is substituted with 1-4 -L-R¹³ groups;

R¹³, at each occurrence, is a) halogen, b) —CN, c) —NO₂, d) oxo, e) —OH, f) —NH₂, g) —NH(C₁₋₁₀ alkyl), h) —N(C₁₋₁₀ alkyl)₂, i) —CHO, j) —C(O)—C₁₋₁₀ alkyl, k) —C(O)OH, l) —C(O)—O(C₁₋₁₀ alkyl), m) —C(O)SH, n) —C(O)—SC₁₋₁₀ alkyl, o) —C(O)NH₂, p) —C(O)NH(C₁₋₁₀ alkyl), q) —C(O)N(C₁₋₁₀ alkyl)₂, r) —C(S)H, s) —C(S)—C₁₋₁₀ alkyl, t) —C(S)NH₂, u) —C(S)NH(C₁₋₁₀ alkyl), v) —C(S)N(C₁₋₁₀ alkyl)₂, w) —C(NH)H, x) —C(NH)(C₁₋₁₀ alkyl), y) —C(NH)NH₂, z) —C(NH)NH(C₁₋₁₀ alkyl), aa) —C(NH)N(C₁₋₁₀ alkyl)₂, ab) —C(NC₁₋₁₀ alkyl)H, ac) —C(NC₁₋₁₀ alkyl)-C₁₋₁₀ alkyl, ad) —C(NC₁₋₁₀ alkyl)NH(C₁₋₁₀ alkyl), ae) —C(NC₁₋₁₀ alkyl)N(C₁₋₁₀ alkyl)₂, af) —S(O)ₘH, ag) —S(O)ₘ—C₁₋₁₀ alkyl, ah) —S(O)₂OH, ai) —S(O)ₘ—OC₁₋₁₀ alkyl, aj) —S(O)ₘNH₂, ak) —S(O)ₘNH(C₁₋₁₀ alkyl), al) —S(O)ₘN(C₁₋₁₀ alkyl)₂, am) —Si(C₁₋₁₀ alkyl)₃, an) a C₁₋₁₀ alkyl group, ao) a C₂₋₁₀ alkenyl group, ap) a C₂₋₁₀ alkynyl group, aq) a C₁₋₁₀ alkoxy group, ar) a C₁₋₁₀ haloalkyl group, as) a C₃₋₁₄ cycloalkyl group, at) a C₆₋₁₄ aryl group, au) a 3-14 membered cycloheteroalkyl group, or av) a 5-14 membered heteroaryl group;

L, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, e) a divalent C₁₋₁₀ alkoxy group, or f) a covalent bond; and

m, at each occurrence, is 0, 1, or 2.

In various embodiments Y is a covalent bond, —CH2-, —C(O)—, or —CH(OH)— and R6 is 1H-indol-3-yl, 2-methyl-1H-indol-3-yl; isopropyl; pyridinyl; phenyl; pyrrolidinyl; 2,3-dihydro-indolyl; 1,3,5-trimethyl-1H-pyrazol-4-yl; 3-phenyl-[1,2,4]oxadiazol yl; 4-phenyl-[1,2,3]triazolyl; 4-pyridinyl-[1,2,3]triazolyl; 4-cyclohexylmethyl-[1,2,3]triazolyl; 4-benzyl-[1,2,3]triazolyl; 4-(1-hydroxy-1-methyl-ethyl)-[1,2,3]triazolyl; 4-(4-hydroxy-tetrahydro-pyran-4-yl)-[1,2,3]triazol; 4-hydroxymethyl-[1,2,3]triazol, 2-indazol-1-yl; 2-pyrazol-1-yl; or 3,5-Bis-trifluoromethyl-pyrazol-1-yl.

Compounds of the present teachings can be selected from the compounds in Table 1.

TABLE 1

| Cpd | | |
|---|---|---|
| No. | Structure | Name |
| 1 | 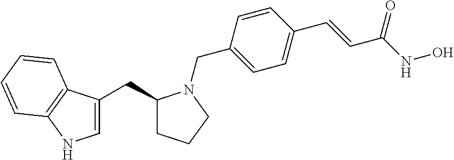 [SMILES: O=C(/C=C/c1ccc(CN2CCC[C@H]2Cc2c[nH]c3ccccc23)cc1)NO] | (E)-N-Hydroxy-3-{4-[(S)-2-(1H-indol- 3-ylmethyl)-pyrrolidin-1-ylmethyl]- phenyl}-acrylamide |
| 2 | 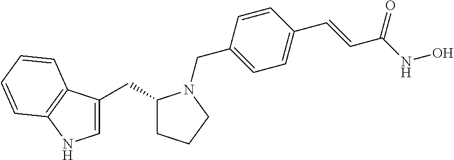 [SMILES: O=C(/C=C/c1ccc(CN2CCC[C@@H]2Cc2c[nH]c3ccccc23)cc1)NO] | (E)-N-Hydroxy-3-{4-[(R)-2-(1H-indol- 3-ylmethyl)-pyrrolidin-1-ylmethyl]- phenyl}-acrylamide |
| 3 | 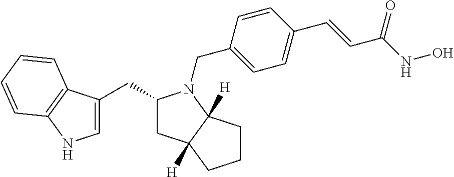 [SMILES: O=C(/C=C/c1ccc(CN2[C@@H](Cc3c[nH]c4ccccc34)C[C@H]3CCC[C@H]32)cc1)NO] | (E)-N-Hydroxy-3-{4-[(2R,3aR,6aR)-2- (2-methyl-1H-indol-3-ylmethyl)- hexahydro-cyclopenta[b]pyrrol-1- ylmethyl]-phenyl}-acrylamide |
| 4 | 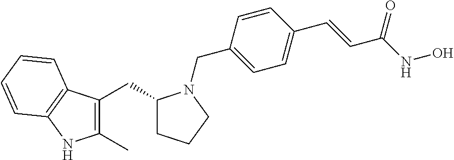 [SMILES: Cc1[nH]c2ccccc2c1C[C@H]1CCCN1Cc1ccc(/C=C/C(=O)NO)cc1] | (E)-N-Hydroxy-3-{4-[(R)-2-(2-methyl- 1H-indol-3-ylmethyl)-pyrrolidin-1- ylmethyl]-phenyl}-acrylamide |
| 5 | 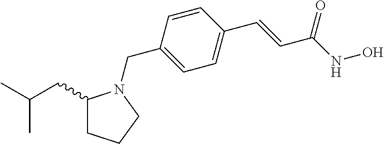 [SMILES: CC(C)CC1CCCN1Cc1ccc(/C=C/C(=O)NO)cc1] | (E)-N-Hydroxy-3-[4-(2-isobutyl- pyrrolidin-1-ylmethyl)-phenyl]- acrylamide |
| 6 | 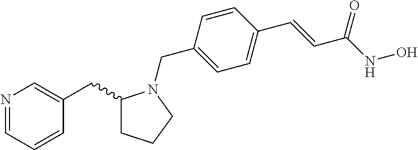 [SMILES: O=C(/C=C/c1ccc(CN2CCCC2Cc2cccnc2)cc1)NO] | (E)-N-Hydroxy-3-[4-(2-pyridin-3- ylmethyl-pyrrolidin-1-ylmethyl)- phenyl]-acrylamide |
| 7 | 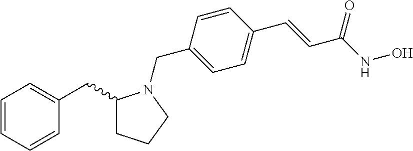 [SMILES: O=C(/C=C/c1ccc(CN2CCCC2Cc2ccccc2)cc1)NO] | (E)-3-[4-(2-Benzyl-pyrrolidin-1- ylmethyl)-phenyl]-N-hydroxy- acrylamide |
| 8 | 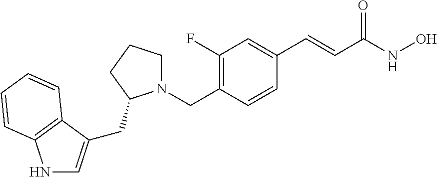 [SMILES: O=C(/C=C/c1ccc(CN2CCC[C@H]2Cc2c[nH]c3ccccc23)c(F)c1)NO] | (E)-3-{3-Fluoro-4-[(S)-2-(1H-indol-3- ylmethyl)-pyrrolidin-1-ylmethyl]- phenyl}-N-hydroxy-acrylamide |
| 9 | 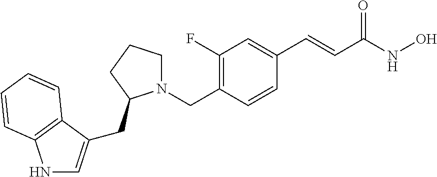 [SMILES: O=C(/C=C/c1ccc(CN2CCC[C@@H]2Cc2c[nH]c3ccccc23)c(F)c1)NO] | (E)-3-{3-Fluoro-4-[(R)-2-(1H-indol-3- ylmethyl)-pyrrolidin-1-ylmethyl]- phenyl}-N-hydroxy-acrylamide |
| 10 | 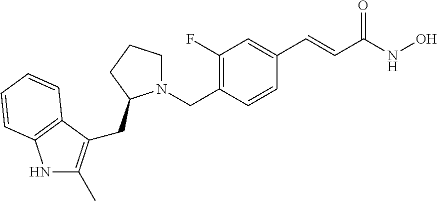 [SMILES: Cc1[nH]c2ccccc2c1C[C@H]1CCCN1Cc1ccc(/C=C/C(=O)NO)cc1F] | (E)-3-{3-Fluoro-4-[(R)-2-(2-methyl- 1H-indol-3-ylmethyl)-pyrrolidin-1- ylmethyl]-phenyl}-N-hydroxy- acrylamide |
| 11 | 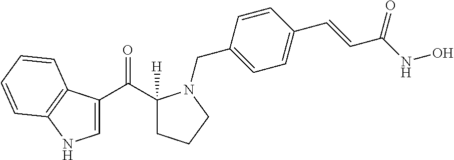 [SMILES: O=C(/C=C/c1ccc(CN2CCC[C@H]2C(=O)c2c[nH]c3ccccc23)cc1)NO] | (E)-N-Hydroxy-3-{4-[(S)-2-(1H- indole-3-carbonyl)-pyrrolidin-1- ylmethyl]-phenyl}-acrylamide |
| 12 | 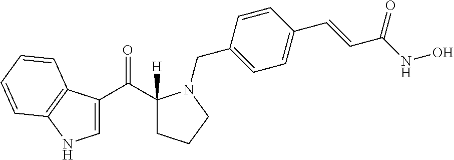 [SMILES: O=C(/C=C/c1ccc(CN2CCC[C@@H]2C(=O)c2c[nH]c3ccccc23)cc1)NO] | (E)-N-Hydroxy-3-{4-[(R)-2-(1H- indole-3-carbonyl)-pyrrolidin-1- ylmethyl]-phenyl}-acrylamide |
| 13 | 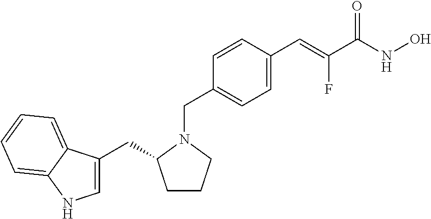 [SMILES: O=C(NO)/C(F)=C/c1ccc(CN2CCC[C@@H]2Cc2c[nH]c3ccccc23)cc1] | (Z)-2-Fluoro-N-hydroxy-3-{4-[(R)-2- (1H-indol-3-ylmethyl)-pyrrolidin-1- ylmethyl]-phenyl}-acrylamide |
| 14 | 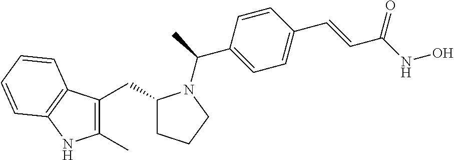 [SMILES: Cc1[nH]c2ccccc2c1C[C@H]1CCCN1[C@@H](C)c1ccc(/C=C/C(=O)NO)cc1] | (E)-N-Hydroxy-3-(4-{(S)-1-[(R)-2-(2- methyl-1H-indol-3-ylmethyl)- pyrrolidin-1-yl]-ethyl}-phenyl)- acrylamide |
| 15 | 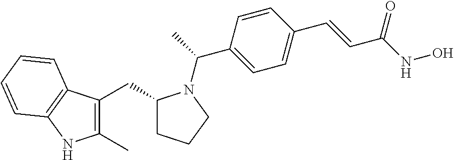 [SMILES: Cc1[nH]c2ccccc2c1C[C@H]1CCCN1[C@H](C)c1ccc(/C=C/C(=O)NO)cc1] | (E)-N-Hydroxy-3-(4-{(R)-1-[(R)-2-(2- methyl-1H-indol-3-ylmethyl)- pyrrolidin-1-yl]-ethyl}-phenyl)- acrylamide |
| 16 | 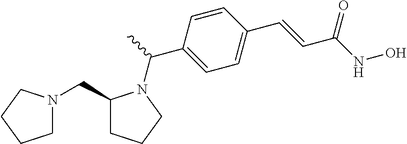 [SMILES: CC(c1ccc(/C=C/C(=O)NO)cc1)N1CCC[C@H]1CN1CCCC1] | (E)-N-Hydroxy-3-{4-[1-((S)-2- pyrrolidin-1-ylmethyl-pyrrolidin-1-yl)- ethyl]-phenyl}-acrylamide |
| 17 | 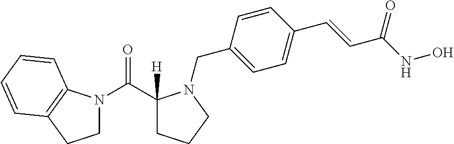 [SMILES: O=C(/C=C/c1ccc(CN2CCC[C@@H]2C(=O)N2CCc3ccccc32)cc1)NO] | (E)-3-{4-[(R)-2-(2,3-Dihydro-indole-1- carbonyl)-pyrrolidin-1-ylmethyl]- phenyl}-N-hydroxy-acrylamide |
| 18 | 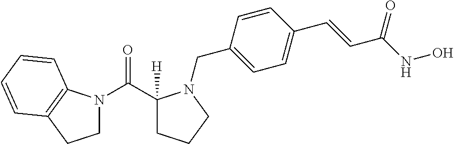 [SMILES: O=C(/C=C/c1ccc(CN2CCC[C@H]2C(=O)N2CCc3ccccc32)cc1)NO] | (E)-3-{4-[(S)-2-(2,3-Dihydro-indole-1- carbonyl)-pyrrolidin-1-ylmethyl]- phenyl}-N-hydroxy-acrylamide |
| 19 | 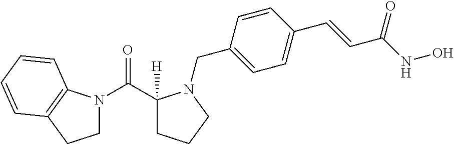 [SMILES: O=C(/C=C/c1ccc(CN2CCC[C@H]2C(=O)N2CCc3ccccc32)cc1)NO] | (E)-3-{4-[(S)-2-(2,3-Dihydro-indol-1- ylmethyl)-pyrrolidin-1-ylmethyl]- phenyl}-N-hydroxy-acrylamide |
| 20 | 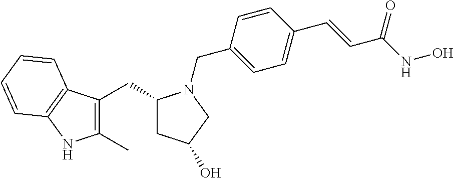 [SMILES: Cc1[nH]c2ccccc2c1C[C@H]1C[C@@H](O)CN1Cc1ccc(/C=C/C(=O)NO)cc1] | (E)-N-Hydroxy-3-{4-[(2S,4R)-4- hydroxy-2-(2-methyl-1H-indol-3- ylmethyl)-pyrrolidin-1-ylmethyl]- phenyl}-acrylamide |
| 21 | 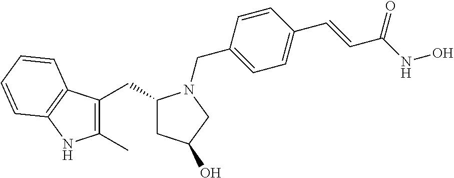 [SMILES: Cc1[nH]c2ccccc2c1C[C@H]1C[C@H](O)CN1Cc1ccc(/C=C/C(=O)NO)cc1] | (E)-N-Hydroxy-3-{4-[(2S,4S)-4- hydroxy-2-(2-methyl-1H-indol-3- ylmethyl)-pyrrolidin-1-ylmethyl]- phenyl}-acrylamide |
| 22 | 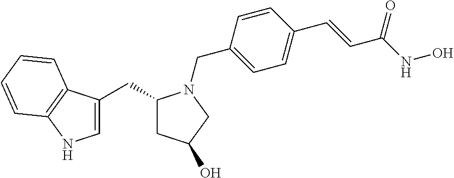 [SMILES: O=C(/C=C/c1ccc(CN2C[C@@H](O)C[C@@H]2Cc2c[nH]c3ccccc23)cc1)NO] | (E)-N-Hydroxy-3-{4-[(2S,4S)-4- hydroxy-2-(1H-indol-3-ylmethyl)- pyrrolidin-1-ylmethyl]-phenyl}- acrylamide |
| 23 | 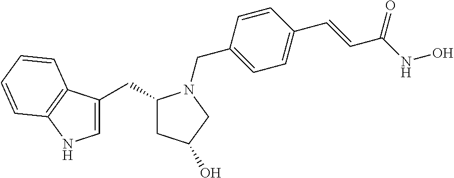 [SMILES: O=C(/C=C/c1ccc(CN2C[C@H](O)C[C@@H]2Cc2c[nH]c3ccccc23)cc1)NO] | (E)-N-Hydroxy-3-{4-[(2S,4R)-4- hydroxy-2-(1H-indol-3-ylmethyl)- pyrrolidin-1-ylmethyl]-phenyl}- acrylamide |
| 24 | 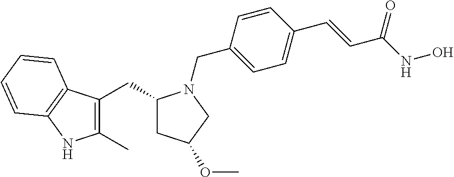 [SMILES: CO[C@@H]1C[C@H](Cc2c(C)[nH]c3ccccc23)N(Cc2ccc(/C=C/C(=O)NO)cc2)C1] | (E)-N-Hydroxy-3-{4-[(2S,4R)-4- methoxy-2-(2-methyl-1H-indol-3- ylmethyl)-pyrrolidin-1-ylmethyl]- phenyl}-acrylamide |
| 25 | 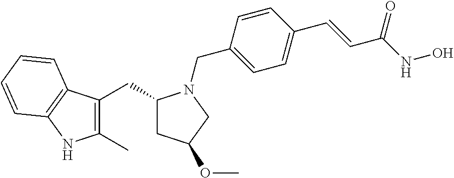 [SMILES: CO[C@H]1C[C@H](Cc2c(C)[nH]c3ccccc23)N(Cc2ccc(/C=C/C(=O)NO)cc2)C1] | (E)-N-Hydroxy-3-{4-[(2S,4S)-4- methoxy-2-(2-methyl-1H-indol-3- ylmethyl)-pyrrolidin-1-ylmethyl]- phenyl}-acrylamide |
| 26 | 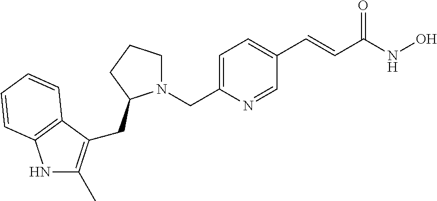 [SMILES: Cc1[nH]c2ccccc2c1C[C@H]1CCCN1Cc1ccc(/C=C/C(=O)NO)cn1] | (E)-N-Hydroxy-3-{6-[(R)-2-(2-methyl- 1H-indol-3-ylmethyl)-pyrrolidin-1- ylmethyl]-pyridin-3-yl}-acrylamide |
| 27 | 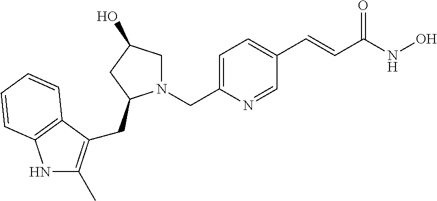 [SMILES: Cc1[nH]c2ccccc2c1C[C@H]1C[C@@H](O)CN1Cc1ccc(/C=C/C(=O)NO)cn1] | (E)-N-Hydroxy-3-{6-[(2S,4R)-4- hydroxy-2-(2-methyl-1H-indol-3- ylmethyl)-pyrrolidin-1-ylmethyl]- pyridin-3-yl}-acrylamide |
| 28 | 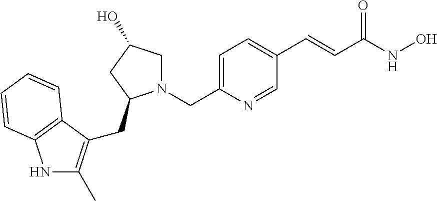 [SMILES: Cc1[nH]c2ccccc2c1C[C@H]1C[C@H](O)CN1Cc1ccc(/C=C/C(=O)NO)cn1] | (E)-N-Hydroxy-3-{6-[(2S,4S)-4- hydroxy-2-(2-methyl-1H-indol-3- ylmethyl)-pyrrolidin-1-ylmethyl]- pyridin-3-yl}-acrylamide |
| 29 | 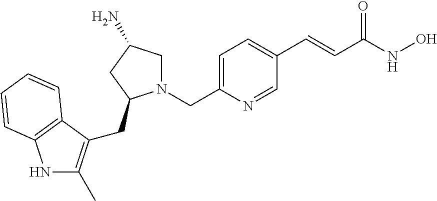 [SMILES: Cc1[nH]c2ccccc2c1C[C@H]1C[C@H](N)CN1Cc1ccc(/C=C/C(=O)NO)cn1] | (E)-3-{6-[(2S,4S)-4-Amino-2-(2- methyl-1H-indol-3-ylmethyl)- pyrrolidin-1-ylmethyl]-pyridin-3-yl}- N-hydroxy-acrylamide |
| 30 | 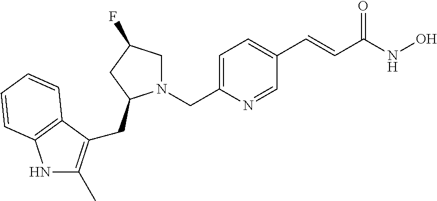 [SMILES: Cc1[nH]c2ccccc2c1C[C@H]1C[C@@H](F)CN1Cc1ccc(/C=C/C(=O)NO)cn1] | (E)-3-{6-[(S)-4-Fluoro-2-(2-methyl- 1H-indol-3-ylmethyl)-pyrrolidin-1- ylmethyl]-pyridin-3-yl}-N-hydroxy- acrylamide |
| 31 | 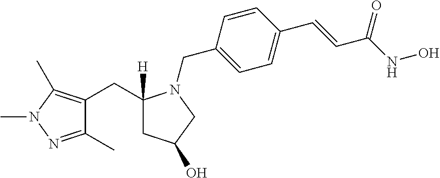 [SMILES: Cc1nn(C)c(C)c1C[C@H]1C[C@H](O)CN1Cc1ccc(/C=C/C(=O)NO)cc1] | (E)-N-Hydroxy-3-{4-[(2S,4S)-4- hydroxy-2-(1,3,5-trimethyl-1H- pyrazol-4-ylmethyl)-pyrrolidin-1- ylmethyl]-phenyl}-acrylamide |
| 32 | 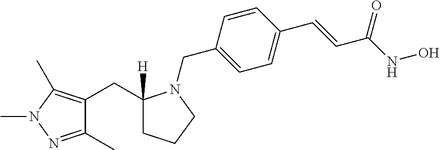 [SMILES: Cc1nn(C)c(C)c1C[C@H]1CCCN1Cc1ccc(/C=C/C(=O)NO)cc1] | (E)-N-Hydroxy-3-{4-[(R)-2-(1,3,5- trimethyl-1H-pyrazol-4-ylmethyl)- pyrrolidin-1-ylmethyl]-phenyl}- acrylamide |
| 33 | 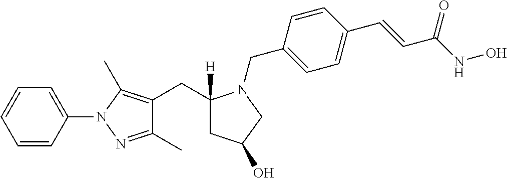 [SMILES: Cc1nn(-c2ccccc2)c(C)c1C[C@H]1C[C@H](O)CN1Cc1ccc(/C=C/C(=O)NO)cc1] | (E)-3-{4-[(2S,4S)-2-(3,5-Dimethyl-1- phenyl-1H-pyrazol-4-ylmethyl)-4- hydroxy-pyrrolidin-1-ylmethyl]- phenyl}-N-hydroxy-acrylamide |
| 34 | 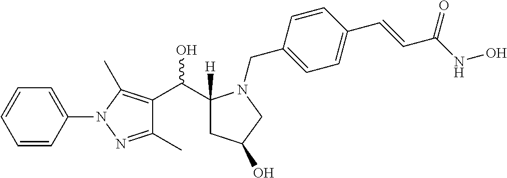 [SMILES: Cc1nn(-c2ccccc2)c(C)c1C(O)[C@H]1C[C@H](O)CN1Cc1ccc(/C=C/C(=O)NO)cc1] | (E)-3-(4-{(2R,4S)-2-[(3,5-Dimethyl-1- phenyl-1H-pyrazol-4-yl)-hydroxy- methyl]-4-hydroxy-pyrrolidin-1- ylmethyl}-phenyl)-N-hydroxy- acrylamide |
| 35 | 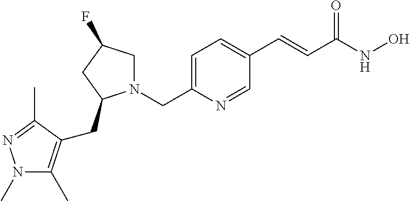 [SMILES: Cc1nn(C)c(C)c1C[C@H]1C[C@@H](F)CN1Cc1ccc(/C=C/C(=O)NO)cn1] | (E)-3-{6-[(2S,4R)-4-Fluoro-2-(1,3,5- trimethyl-1H-pyrazol-4-ylmethyl)- pyrrolidin-1-ylmethyl]-pyridin-3-yl}- N-hydroxy-acrylamide |
| 36 | 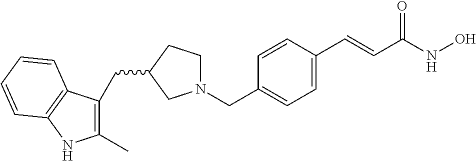 [SMILES: Cc1[nH]c2ccccc2c1CC1CCN(Cc2ccc(/C=C/C(=O)NO)cc2)C1] | racemic (E)-N-Hydroxy-3-{4-[3-(2- methyl-1H-indol-3-yl)-pyrrolidin-1- ylmethyl]-phenyl}-acrylamide |
| 37 | 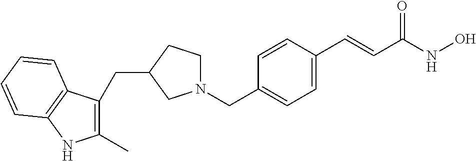 [SMILES: Cc1[nH]c2ccccc2c1CC1CCN(Cc2ccc(/C=C/C(=O)NO)cc2)C1] | chiral (E)-N-Hydroxy-3-{4-[3-(2- methyl-1H-indol-3-yl)-pyrrolidin-1- ylmethyl]-phenyl}-acrylamide |
| 38 | 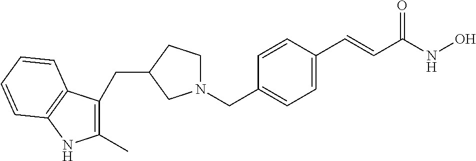 [SMILES: Cc1[nH]c2ccccc2c1CC1CCN(Cc2ccc(/C=C/C(=O)NO)cc2)C1] | chiral (E)-N-Hydroxy-3-{4-[3-(2- methyl-1H-indol-3-yl)-pyrrolidin-1- ylmethyl]-phenyl}-acrylamide |
| 39 | 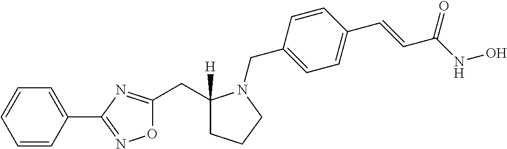 [SMILES: O=C(/C=C/c1ccc(CN2CCC[C@@H]2Cc2nc(-c3ccccc3)no2)cc1)NO] | (E)-N-Hydroxy-3-{4-[(R)-2-(3-phenyl- [1,2,4]oxadiazol-5-ylmethyl)- pyrrolidin-1-ylmethyl]-phenyl}- acrylamide |
| 40 | 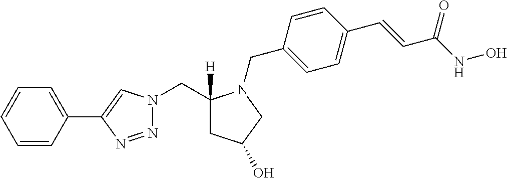 [SMILES: O=C(/C=C/c1ccc(CN2C[C@H](O)C[C@@H]2Cn2cc(-c3ccccc3)nn2)cc1)NO] | (E)-N-Hydroxy-3-{4-[(2R,4R)-4- hydroxy-2-(4-phenyl-[1,2,3]triazol-1- ylmethyl)-pyrrolidin-1-ylmethyl]- phenyl}-acrylamide |
| 41 | 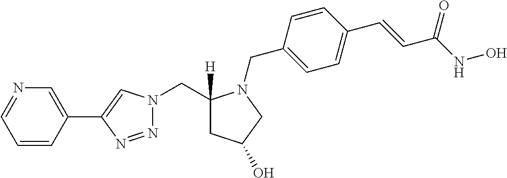 [SMILES: O=C(/C=C/c1ccc(CN2C[C@H](O)C[C@@H]2Cn2cc(-c3cccnc3)nn2)cc1)NO] | (E)-N-Hydroxy-3-{4-[(2R,4R)-4- hydroxy-2-(4-pyridin-3-yl- [1,2,3]triazol-1-ylmethyl)-pyrrolidin-1- ylmethyl]-phenyl}-acrylamide |
| 42 | 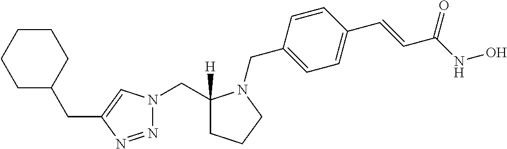 [SMILES: O=C(/C=C/c1ccc(CN2CCC[C@@H]2Cn2cc(CC3CCCCC3)nn2)cc1)NO] | (E)-3-{4-[(R)-2-(4-Cyclohexylmethyl- [1,2,3]triazol-1-ylmethyl)-pyrrolidin-1- ylmethyl]-phenyl}-N-hydroxy- acrylamide |
| 43 | 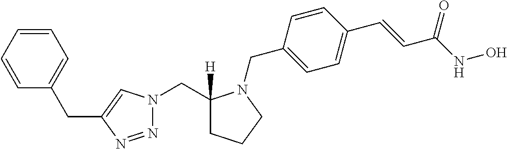 [SMILES: O=C(/C=C/c1ccc(CN2CCC[C@@H]2Cn2cc(Cc3ccccc3)nn2)cc1)NO] | (E)-3-{4-[(R)-2-(4-Benzyl- [1,2,3[triazol-1-ylmethyl)-pyrrolidin-1- ylmethyl]-phenyl}-N-hydroxy- acrylamide |
| 44 | 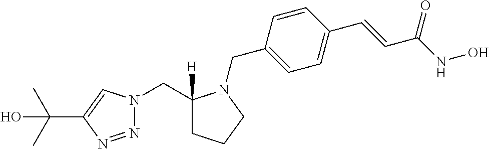 [SMILES: CC(C)(O)c1cn(C[C@H]2CCCN2Cc2ccc(/C=C/C(=O)NO)cc2)nn1] | (E)-N-Hydroxy-3-(4-{(R)-2-[4-(1- hydroxy-1-methyl-ethyl)-[1,2,3]triazol- 1-ylmethyl]-pyrrolidin-1-ylmethyl}- phenyl)-acrylamide |
| 45 | 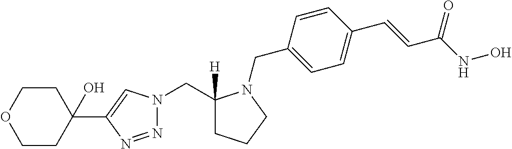 [SMILES: O=C(/C=C/c1ccc(CN2CCC[C@@H]2Cn2cc(C3(O)CCOCC3)nn2)cc1)NO] | (E)-N-Hydroxy-3-(4-{(R)-2-[4-(4- hydroxy-tetrahydro-pyran-4-yl)- [1,2,3]-triazol-1-ylmethyl]-pyrrolidin-1- ylmethyl}-phenyl)-acrylamide |
| 46 | 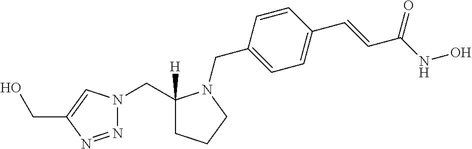 [SMILES: O=C(/C=C/c1ccc(CN2CCC[C@@H]2Cn2cc(CO)nn2)cc1)NO] | (E)-N-Hydroxy-3-{4-[(R)-2-(4- hydroxymethyl-[1,2,3]triazol-1- ylmethyl)-pyrrolidin-1-ylmethyl]- phenyl}-acrylamide |
| 47 | 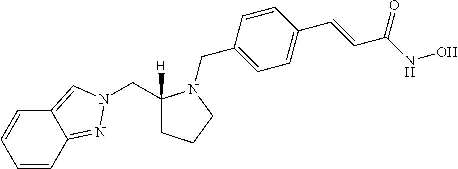 [SMILES: O=C(/C=C/c1ccc(CN2CCC[C@@H]2Cn2cc3ccccc3n2)cc1)NO] | (E)-N-Hydroxy-3-[4-((R)-2-indazol-1- ylmethyl-pyrrolidin-1-ylmethyl)- phenyl]-acrylamide |
| 48 | 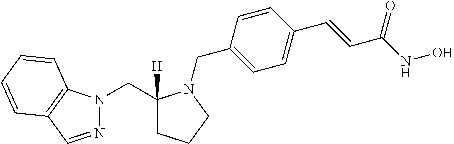 [SMILES: O=C(/C=C/c1ccc(CN2CCC[C@@H]2Cn2ncc3ccccc32)cc1)NO] | (E)-N-Hydroxy-3-[4-((R)-2-indazol-2- ylmethyl-pyrrolidin-1-ylmethyl)- phenyl]-acrylamide |
| 49 | 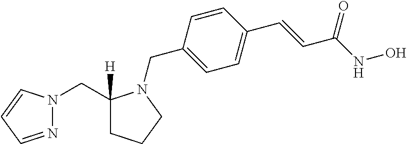 [SMILES: O=C(/C=C/c1ccc(CN2CCC[C@@H]2Cn2cccn2)cc1)NO] | (E)-N-Hydroxy-3-[4-((R)-2-pyrazol-1- ylmethyl-pyrrolidin-1-ylmethyl)- phenyl]-acrylamide |
| 50 | 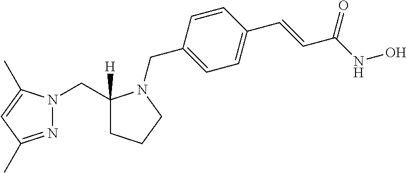 [SMILES: Cc1cc(C)n(C[C@H]2CCCN2Cc2ccc(/C=C/C(=O)NO)cc2)n1] | (E)-3-{4-[(R)-2-(3,5-Dimethyl- pyrazol-1-ylmethyl)-pyrrolidin-1- ylmethyl]-phenyl}-N-hydroxy- acrylamide |
| 51 | 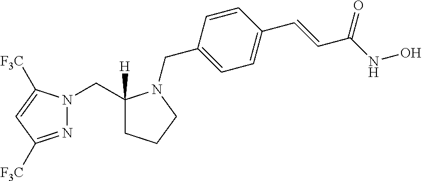 [SMILES: O=C(/C=C/c1ccc(CN2CCC[C@@H]2Cn2nc(C(F)(F)F)cc2C(F)(F)F)cc1)NO] | (E)-3-{4-[(R)-2-(3,5-Bis- trifluoromethyl-pyrazol-1-ylmethyl)- pyrrolidin-1-ylmethyl]-phenyl}-N- hydroxy-acrylamide |
| 52 | 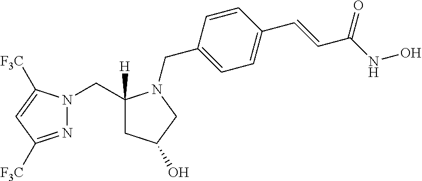 [SMILES: O=C(/C=C/c1ccc(CN2C[C@H](O)C[C@@H]2Cn2nc(C(F)(F)F)cc2C(F)(F)F)cc1)NO] | (E)-3-{4-[(2R,4R)-2-(3,5-Bis- trifluoromethyl-pyrazol-1-ylmethyl)-4- hydroxy-pyrrolidin-1-ylmethyl]- phenyl}-N-hydroxy-acrylamide |
| 53 | 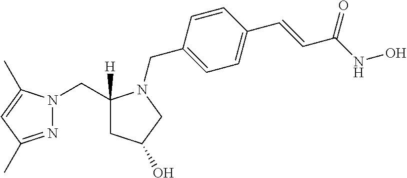 [SMILES: Cc1cc(C)n(C[C@H]2C[C@@H](O)CN2Cc2ccc(/C=C/C(=O)NO)cc2)n1] | (E)-3-{4-[(2R,4R)-2-(3,5-Dimethyl- pyrazol-1-ylmethyl)-4-hydroxy- pyrrolidin-1-ylmethyl]-phenyl}-N- hydroxy-acrylamide |

Also provided in accordance with the present teachings are prodrugs of the compounds disclosed herein. As used herein, “prodrug” refers to a compound (“parent compound”) having a moiety that produces, generates, or releases a compound of the present teachings (“active compound”) when administered to a mammalian subject. Prodrugs can be prepared by modifying functional groups present in the active compounds in such a way that the modifications can be removed, either by routine manipulation or in vivo, from the parent compounds. Examples of prodrugs include compounds that contain one or more molecular moieties that are appended to a hydroxyl, amino, sulfhydryl, or carboxyl group of the active compounds, and that, when administered to a mammalian subject, is/are cleaved in vivo to form the free hydroxyl, amino, sulfhydryl, or carboxyl group, respectively, and to release the active compound. Examples of prodrugs can include acetate, formate, and benzoate derivatives of hydroxy and amino functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Ester forms of the compounds according to the present teachings include pharmaceutically acceptable esters known in the art that can be metabolized into the free acid form, such as a free carboxylic acid form, in a mammal body. Examples of such esters include alkyl esters (e.g., alkyls of 1 to 10 carbon atoms), cycloalkyl esters (e.g., cycloalkyls of 3-10 carbon atoms), aryl esters (e.g., aryls of 6-14 carbon atoms, including of 6-10 carbon atoms), and heterocyclic analogues thereof (e.g., heterocyclics of 3-14 ring atoms, 1-3 of which can be selected from O, N, and S) and the alcoholic residue can carry further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, hexyl esters, cyclopropylmethyl esters, and benzyl esters, C₃₋₁₀ cycloalkyl esters, such as cyclopropyl esters, cyclobutyl esters, cyclopentyl esters, and cyclohexyl esters, or aryl esters, such as phenyl esters and tolyl ester.

Pharmaceutically acceptable salts of compounds of the present teachings, which can have an acidic moiety, can be formed using organic or inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di-, or tri-lower alkylamine (e.g., ethyl-tert-butylamine, diethylamine, diisopropylamine, triethylamine, tributylamine, or dimethylpropylamine), or a mono-, di-, or trihydroxy lower alkylamine (e.g., mono-, di- or triethanolamine). Non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from any of the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, as well as other known pharmaceutically acceptable acids.

In another aspect, the present teachings provide pharmaceutical compositions including at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington: The Science and Practice of Pharmacy, 20th edition, Alfonoso R. Gennaro (ed.), Lippincott Williams & Wilkins, Baltimore, Md. (2000), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be useful for inhibiting a deacetylase in a cell. Accordingly, another aspect of the present teachings includes a method of contacting a cell with one or more compounds of the present teachings (or a salt, hydrate, ester, or prodrug thereof) or a composition that includes one or more compounds of the present teachings. In certain embodiments, the composition can further include one or more pharmaceutically acceptable carrier or excipients.

Compounds of the present teachings can be useful for the treatment, inhibition, prevention, or diagnosis of a pathological condition or disorder in a mammal, for example, a human. Accordingly, another aspect of the present teachings includes a method of providing to a mammal a compound of the present teachings (or its pharmaceutically acceptable salt, hydrate, ester, or prodrug) or a pharmaceutical composition that includes one or more compounds of the present teachings in combination or association with a pharmaceutically acceptable carrier. Compounds of the present teachings can be administered alone or in combination with other therapeutically effective compounds or therapies for the treatment, inhibition, prevention, or diagnosis of the pathological condition or disorder. As used herein, “therapeutically effective” refers to a substance or an amount that elicits a desirable biological activity or effect.

In various embodiments, the present teachings can further include use of the compounds disclosed herein as active therapeutic substances for the treatment or inhibition of a pathological condition or disorder, for example, a condition mediated wholly or in part by one or more deacetylases, such as an undesired proliferative condition; a neurodegenerative disease, including Alzheimer's disease, Hungtington's disease, Rubenstein-Taybis syndrome, Parkinson's disease, muscular dystrophy, spinal muscular atrophy, Rett's syndrome, and the like; a cardiovascular disease, including heart failure, cardiac hypertrophy, thrombosis, and the like; an autoimmune disease, including Lupus, atherosclerosis, scleroderma, and the like; an inflammatory disorder, including arthritis and arthritic conditions (e.g., osteoarthritis, rheumatoid arthritis, and the like), and other chronic inflammatory disorders (e.g., chronic asthma, arterial or post-transplantational atherosclerosis, endometriosis, and the like); an undesired immunological process; stroke; and an fungal infection. In some embodiments, the undesired proliferative condition includes a cancer (e.g., brain cancer, kidney cancer, liver cancer, adrenal gland cancer, bladder cancer, breast tumor, stomach cancer including gastric tumors, esophagus cancer, ovarian cancer, colon cancer, rectum cancer, prostate cancer, pancrea cancer, lung cancer including small cell lung cancer, vagina cancer, thyroid cancer, sarcoma, glioblastomas, multiple myeloma, gastrointestinal cancer, lung cancer, colon cancer, breast cancer, ovarian cancer, bladder cancer), a tumor, a fibrosis, and the like; a neoplasia, including mammary carcinoma, leukemia, and the like; and an epidermal hyperproliferation, including psoriasis, prostate hyperplasia, and the like. In certain embodiments, the present teachings can provide methods of treating these pathological conditions and disorders using the compounds described herein. As used herein, “treating” refers to partially or completely alleviating and/or ameliorating the condition or symptoms thereof. In particular embodiments, the methods can include identifying a mammal having a pathological condition or disorder mediated by deacetylases, and providing to the mammal a therapeutically effective amount of a compound as described herein. In some embodiments, the method can include administering to a mammal a pharmaceutical composition that can include a compound disclosed herein in combination or association with a pharmaceutically acceptable carrier.

In various embodiments, the present teachings can further include use of the compounds disclosed herein as active therapeutic substances for the prevention of a pathological condition or disorder, for example, a condition mediated wholly or in part by one or more deacetylases, such as an undesired proliferative condition; a neurodegenerative disease, including Alzheimer's disease, Hungtington's disease, Rubenstein-Taybis syndrome, Parkinson's disease, muscular dystrophy, spinal muscular atrophy, Rett's syndrome, and the like; a cardiovascular disease, including heart failure, cardiac hypertrophy, thrombosis, and the like; an autoimmune disease, including Lupus, atherosclerosis, scleroderma, and the like; an inflammatory disorder, including arthritis and arthritic conditions (e.g., osteoarthritis, rheumatoid arthritis, and the like), and other chronic inflammatory disorders (e.g., chronic asthma, arterial or post-transplantational atherosclerosis, endometriosis, and the like); an undesired immunological process; stroke; and an fungal infection. In some embodiments, the undesired proliferative condition includes a cancer (e.g., brain cancer, kidney cancer, liver cancer, adrenal gland cancer, bladder cancer, breast tumor, stomach cancer including gastric tumors, esophagus cancer, ovarian cancer, colon cancer, rectum cancer, prostate cancer, pancrea cancer, lung cancer including small cell lung cancer, vagina cancer, thyroid cancer, sarcoma, glioblastomas, multiple myeloma, gastrointestinal cancer, lung cancer, colon cancer, breast cancer, ovarian cancer, bladder cancer), a tumor, a fibrosis, and the like; a neoplasia, including mammary carcinoma, leukemia, and the like; and an epidermal hyperproliferation, including psoriasis, prostate hyperplasia, and the like. In some embodiments, the present teachings can provide methods of preventing these pathological conditions and disorders using the compounds described herein. In certain embodiments, the methods can include identifying a mammal that could potentially have a pathological condition or disorder mediated by deacetylases, and providing to the mammal a therapeutically effective amount of a compound as described herein. In some embodiments, the method can include administering to a mammal a pharmaceutical composition that can include a compound disclosed herein in combination or association with a pharmaceutically acceptable carrier.

Cardiac hypertrophy in response to an increased workload imposed on the heart is a fundamental adaptive mechanism. It is a specialized process reflecting a quantitative increase in cell size and mass (rather than cell number) as the result of any or a combination of neural, endocrine or mechanical stimuli. Hypertension, another factor involved in cardiac hypertrophy, is a frequent precursor of congestive heart failure. When heart failure occurs, the left ventricle usually is hypertrophied and dilated and indices of systolic function, such as ejection fraction, are reduced. Clearly, the cardiac hypertrophic response is a complex syndrome and the elucidation of the pathways leading to cardiac hypertrophy will be beneficial in the treatment of heart disease resulting from a various stimuli.

In an embodiment, there is provided a method of preventing pathologic cardiac hypertrophy and heart failure with the compounds of the present invention. The method includes administering to the patient a histone deacetylase inhibitor. Administration may comprise intravenous, oral, transdermal, sustained release, suppository, or sublingual administration. The patient at risk may exhibit one or more of long standing uncontrolled hypertension, uncorrected valvular disease, chronic angina and/or recent myocardial infarction.

In one embodiment of the present invention, methods for the treatment of cardiac hypertrophy utilizing HDAC inhibitors are provided. For the purposes of the present application, treatment comprises reducing one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of HDAC inhibitors may prevent cardiac hypertrophy and its associated symptoms from arising.

Treatment regimens would vary depending on the clinical situation. However, long term maintenance would appear to be appropriate in most circumstances. It also may be desirable treat hypertrophy with HDAC inhibitors intermittently, such as within brief window during disease progression. At present, testing indicates that the optimal dosage for an HDAC inhibitor will be the maximal dose before significant toxicity occurs.

In another embodiment, it is envisioned to use an HDAC inhibition in combination with other therapeutic modalities. Thus, in addition to the therapies described above, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors.

In an embodiment, the cardiovascular indications for which the HDAC inhibitors may be used include: diastolic dysfunction, myocardial Infarction (systolic dysfunction), inhibition of overall cardiac remodeling in both acute and chronic heart failure conditions, adriamycin induced cardiotoxicity, inducing cardioprotection from ischemic events, and for the use of hemorrhagic shock and resuscitation.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders, tablet-disintegrating agents, or encapsulating materials. The compounds can be formulated in conventional manner, for example, in a manner similar to that used for known HDAC inhibitors. Oral formulations containing an active compound disclosed herein can include any conventionally used oral form, including tablets, capsules, buccal forms, troches, lozenges and oral liquids, suspensions, and solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) optionally with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending agents, or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also consist of administering an active compound in water or fruit juice, containing appropriate solubilizers or emulsifiers as needed.

Liquid carriers can be used in preparing solutions, suspensions, emulsions, syrups, and elixirs. An active compound described herein can be dissolved or suspended in a pharmaceutically acceptable liquid carrier such as water, an organic solvent, a mixture thereof, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal, or subcutaneous injection. Sterile solutions can also be administered intravenously. Compositions for oral administration can be in either liquid or solid form.

The pharmaceutical composition can be in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts, hydrates, and esters thereof, in lotions, creams, foams, patches, suspensions, solutions, and suppositories (rectal and vaginal).

When administered for the treatment or inhibition of a particular pathologic condition or disorder, it is understood that an effective dosage can vary depending upon the particular compound utilized, the mode of administration, and/or severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. In preventive applications, a compound of the present teachings can be provided to a patient that can suffer from a disease in an amount sufficient to prevent or at least delay the symptoms of the disease and its complications. The dosage to be used in the treatment of a specific individual typically must be subjectively determined by the attending physician. The variables involved include the specific condition and its state as well as the size, age and response pattern of the patient.

In some cases, for example, those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these active compounds or pharmaceutically acceptable salts, hydrates, or esters thereof can be prepared in water mixed with a suitable surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds of the present teachings can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal). Topical formulations that deliver active compound(s) through the epidermis can be useful for localized treatment of a pathologic condition or disorder.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams, ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices known in the literature are also contemplated.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can also be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound disclosed herein with other agents effective in the treatment of the target disease. For proliferative diseases, other active compounds (i.e., other active ingredients or agents) effective in their treatment, and particularly in the treatment of cancers and tumors, can be administered with active compounds of the present teachings. The other agents can be administered at the same time or at different times than the compounds disclosed herein.

The compounds of the present teachings can be prepared in accordance with the procedures outlined in the scheme below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the preparation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatograpy (HPLC), gas chromatograph (GC), or thin layer chromatography.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, the entire disclosure of which is incorporated by reference herein for all purposes.

The reactions described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

### Example 1

### Preparation of (S,E)-3-(4-((2-((1H-indol-3-yl)methyl)pyrrolidin-1-ylmethyl)phenyl)-N-hydroxyacrylamide (1)

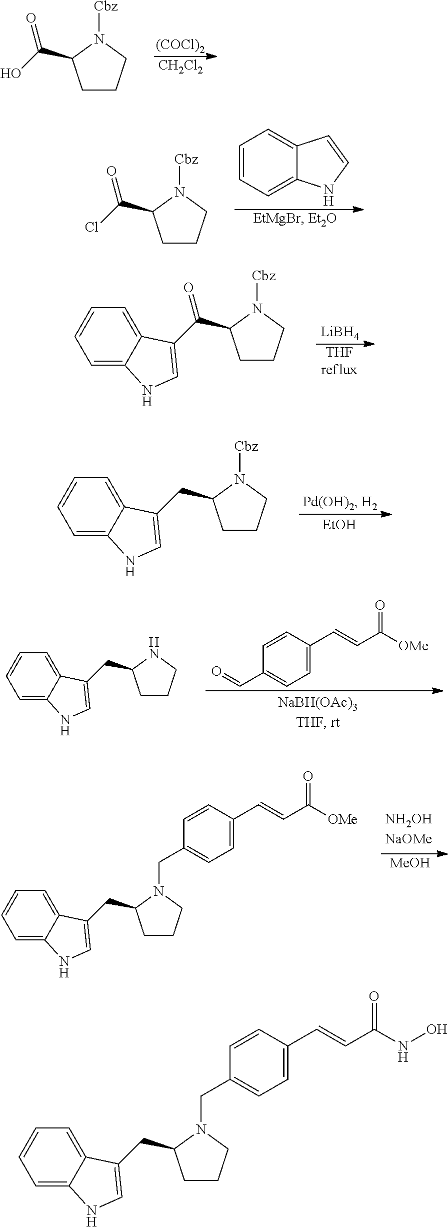

### Step a: Preparation of (S)-2-chlorocarbonyl-pyrrolidine-1-carboxylic acid benzyl ester

To a solution of (S)-pyrrolidine-1,2-dicarboxylic acid 1-benzyl ester (18.9 g, 75.8 mmol) in dichloromethane (42 mL) is added a few drops of N,N-dimethylformamide and oxalyl chloride (14.5 g, 114 mmol) slowly. The reaction mixture is stirred for 1.5 h and monitored by LC-MS. The solvent is removed under reduced pressure and the crude product is dried in vacuo and used in the subsequent step without further purification.

### Step b: Preparation of (S)-2-(1H-indole-3-carbonyl)-pyrrolidine-1-carboxylic acid benzyl ester

To a well-stirred solution of 1H-indole (8.9 g, 75.8 mmol) in anhydrous diethyl ether (303 mL) is added ethyl magnesium bromide (24.2 mL, 75.8 mmol, 3.13 M in diethyl ether) dropwise. The reaction is refluxed for 1.5 h and cooled to room temperature. A solution of (S)-2-chlorocarbonyl-pyrrolidine-1-carboxylic acid benzyl ester (75.8 mmol) in diethyl ether (19 mL) is added slowly. The reaction mixture is stirred for another hour at room temperature under nitrogen, quenched by addition of a saturated solution of sodium bicarbonate (150 mL), and extracted three times with 150 mL of ethyl acetate. The organic layers are combined, washed with a saturated solution of sodium chloride (150 mL), dried with anhydrous magnesium sulfate, filtered, and concentrated in vacuo. The residue is purified via a silica gel column chromatography (20-100% ethyl acetate/heptanes) to provide (S)-2-(1H-indole-3-carbonyl)-pyrrolidine-1-carboxylic acid benzyl ester as a white solid (4.57 g, 17%).

### Step c: Preparation of (S)-2-(1H-indol-3-ylmethyl)-pyrrolidine-1-carboxylic acid benzyl ester

To a solution of (S)-2-(1H-indole-3-carbonyl)-pyrrolidine-1-carboxylic acid benzyl ester (2.0 g, 5.74 mmol) in anhydrous tetrahydrofuran (44.2 mL) is added a lithium borohydride solution (11.5 mL, 23 mmol, 2.0 M in tetrahydrofuran) slowly. The resulting reaction is refluxed under nitrogen for 4 hours, cooled to 0° C., and quenched with methanol (9 mL) slowly. The resulting mixture is stirred for another hour and a saturated solution of sodium bicarbonate (25 mL) is added. The mixture is extracted three times with ethyl acetate (60 mL) and the organic layers are combined, washed with a saturated solution of sodium chloride (150 mL), dried with anhydrous magnesium sulfate, filtered, and concentrated in vacuo. The residue is purified via silica gel chromatography (20-100% ethyl acetate/heptanes) to provide (S)-2-(1H-indol-3-ylmethyl)-pyrrolidine-1-carboxylic acid benzyl ester as a white sticky powder (1.03 g, 54%).

### Step d: Preparation of 3-(S)-1-pyrrolidin-2-ylmethyl-1H-indole

A solution of (S)-2-(1H-indol-3-ylmethyl)-pyrrolidine-1-carboxylic acid benzyl ester (1.03 g, 3.1 mmol) in ethyl alcohol (5.13 mL) is stirred under hydrogen at atmospheric pressure for 12 h in the presence of palladium hydroxide (0.1 weight equivalent) and monitored by LC-MS. The reaction mixture is filtered through Celite and the solvent is removed under reduced pressure to give the title compound as a tan stick solid (599 mg, 97%).

### Step e: Preparation of (E)-3-{4-[(S)-2-(1H-indol-3-ylmethyl)-pyrrolidin-1-ylmethyl]-phenyl}-acrylic acid methyl ester

A solution of 3-(S)-1-pyrrolidin-2-ylmethyl-1H-indole (242 mg, 1.21 mmol) and (E)-3-(4-formyl-phenyl)-acrylic acid methyl ester (192 mg, 1.0 mmol) in tetrahydrofuran (3.4 mL) is stirred for 1 h and sodium triacetoxyborohydride (278 mg, 1.31 mmol) is added. The resulting reaction is stirred 4 hours, quenched by addition of a saturated solution of sodium bicarbonate (10 mL), and extracted three times with 30 mL of ethyl acetate. The organic layers are combined, washed with a saturated solution of sodium chloride (20 mL), dried with magnesium sulfate, filtered, and concentrated in vacuo. The residue is purified via silica gel chromatography (20-100% ethyl acetate/heptanes) to provide (E)-3-{4-[(S)-2-(1H-indol-3-ylmethyl)-pyrrolidin-1-ylmethyl]-phenyl}-acrylic acid methyl ester as a white powder (198 mg, 53% yield).

### Step f: Preparation of (E)-N-hydroxy-3-{4-[(S)-2-(1H-indol-3-ylmethyl)-pyrrolidin-1-ylmethyl]-phenyl}-acrylamide (1)

To a cooled (0° C.) solution of (E)-3-{4-[(S)-2-(1H-indol-3-ylmethyl)-pyrrolidin-1-ylmethyl]-phenyl}-acrylic acid methyl ester (101 mg, 0.27 mmol) in methanol (0.5 mL) are added hydroxyamine (178 uL, 2.7 mmol, 50% in water) and sodium methoxide (292 uL, 1.35 mmol, 25% in methanol) and the mixture is stirred for 15 minutes and neutralized to pH 8 by addition of 1N hydrochloric acid. The precipitate is collected by filtration, washed with water, and dried in vacuum oven overnight to give (E)-N-hydroxy-3-{4-[(S)-2-(1H-indol-3-ylmethyl)-pyrrolidin-1-ylmethyl]-phenyl}-acrylamide (1) as a white powder (50 mg, 50%). HRMS: 376.2025.

Following procedures analogous to those described in Example 1, the following compounds are prepared:

| Cpd | Name | MS |
|---|---|---|
| 2 | (E)-N-Hydroxy-3-{4-[(R)-2-(1H-indol-3-ylmethyl)-pyrrolidin-1-ylmethyl]- | 376.2016 |
| | phenyl}-acrylamide | |
| 4 | (E)-N-Hydroxy-3-{4-[(R)-2-(2-methyl-1H-indol-3-ylmethyl)-pyrrolidin-1- | 390.2180 |
| | ylmethyl]-phenyl}-acrylamide | |
| 5 | (E)-N-Hydroxy-3-[4-(2-isobutyl-pyrrolidin-1-ylmethyl)-phenyl]-acrylamide | 303.4 |
| 6 | (E)-N-Hydroxy-3-[4-(2-pyridin-3-ylmethyl-pyrrolidin-1-ylmethyl)-phenyl]- | 338.1864 |
| | acrylamide | |
| 7 | (E)-3-[4-(2-Benzyl-pyrrolidin-1-ylmethyl)-phenyl]-N-hydroxy-acrylamide | 337.4 |

### Example 2

### Preparation of (E)-N-hydroxy-3-{4-[(2R,3aR,6aR)-2-(2-methyl-1H-indol-3-ylmethyl)-hexahydro-cyclopenta[b]pyrrol-1-ylmethyl]-phenyl}-acrylamide (3)

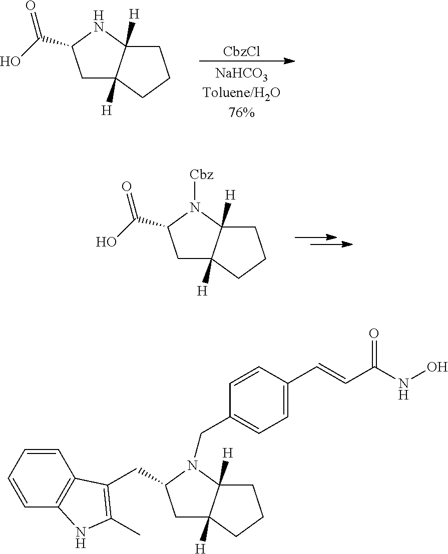

(2R,3aR,6aR)-Octahydro-cyclopenta[b]pyrrole-2-carboxylic acid (2.37 g, 15.3 mmol) and sodium bicarbonate (3.2 g, 38 mmol) are dissolved in water (33 mL) and a solution of benzyl chloroformate (2.5 mL, 3.0 g, 17.6 mmol) in toluene (8 mL) is added over a period of 15 minutes. The resulting mixture is stirred at room temperature for 16 hours and the organic phase is separated from the aqueous layer, which is extracted with ether (4×50 mL), cooled in an ice bath, and acidified to pH 2 with concentrated hydrochloric acid. The resulting oily product is extracted into ethyl acetate (5×50 mL) and the combined organic extracts are dried over magnesium sulfate and concentrated to provide (2R,3aR,6aR)-1-(benzyloxycarbonyl)-octahydrocyclopenta[b]pyrrole-2-carboxylic acid (3.37 g, 76%) as a viscous oil. LCMS: 290.1.

Following procedures analogous to those described in Example 1, (E)-N-hydroxy-3-{4-[(2R,3aR,6aR)-2-(2-methyl-1H-indol-3-ylmethyl)-hexahydro-cyclopenta[b]pyrrol-1-ylmethyl]-phenyl}-acrylamide is prepared. FIRMS: 430.2503.

### Example 3

### Preparation of (E)-3-(3-Fluoro-4-formyl-phenyl)-acrylic acid methyl ester

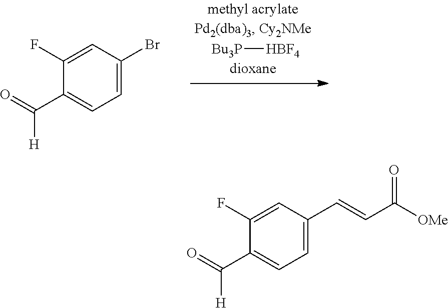

A mixture of 4-bromo-2-fluoro-benzaldehyde (2.50 g, 12.3 mmol), N-methyldicyclohexylamine (3.1 mL, 14.7 mmol), tri-(tert-butyl)phosphoine-tetrafluoroborate (140 mg, 0.48 mmol) and Pd₂(dba)₃ (110 mg, 0.12 mmol) in 1,4-dioxane (8 mL) is sealed in a dry microwave vial and stirred for 30 minutes under N₂. Methyl acrylate (2.2 mL, 24.6 mmol) is added into the vial and the reaction is heated at 100° C. for 30 minutes in a microwave reactor, cooled to room temperature and filtered through a Celite pad, which is rinsed with ethyl acetate. The filtrate and washes are combined and concentrated and the residue is purified by a silica gel column chromatography (ethyl acetate/heptane) to give (E)-3-(3-fluoro-4-formyl-phenyl)-acrylic acid methyl ester (4.0 g, 80% yield). LCMS 209 (M+1).

Following procedures analogous to those described in Example 1, the following compounds are prepared:

| Cpd | Name | MS |
|---|---|---|
| 8 | (E)-3-{3-Fluoro-4-[(S)-2-(1H-indol-3-ylmethyl)-pyrrolidin-1-ylmethyl]- | 394.1919 |
| | phenyl}-N-hydroxy-acrylamide | |
| 9 | (E)-3-{3-Fluoro-4-[(R)-2-(1H-indol-3-ylmethyl)-pyrrolidin-1-ylmethyl]- | 394.1903 |
| | phenyl}-N-hydroxy-acrylamide | |
| 10 | (E)-3-{3-Fluoro-4-[(R)-2-(2-methyl-1H-indol-3-ylmethyl)-pyrrolidin-1- | 408.2079 |
| | ylmethyl]-phenyl}-N-hydroxy-acrylamide | |
| 11 | (E)-N-Hydroxy-3-{4-[(S)-2-(1H-indole-3-carbonyl)-pyrrolidin-1-ylmethyl]- | 390.1804 |
| | phenyl}-acrylamide | |
| 12 | (E)-N-Hydroxy-3-{4-[(R)-2-(1H-indole-3-carbonyl)-pyrrolidin-1-ylmethyl]- | 390.1818 |
| | phenyl}-acrylamide | |

### Example 4

### Preparation of (Z)-2-fluoro-N-hydroxy-3-{4-[(R)-2-(1H-indol-3-ylmethyl)-pyrrolidin-1-ylmethyl]-phenyl}-acrylamide (13)

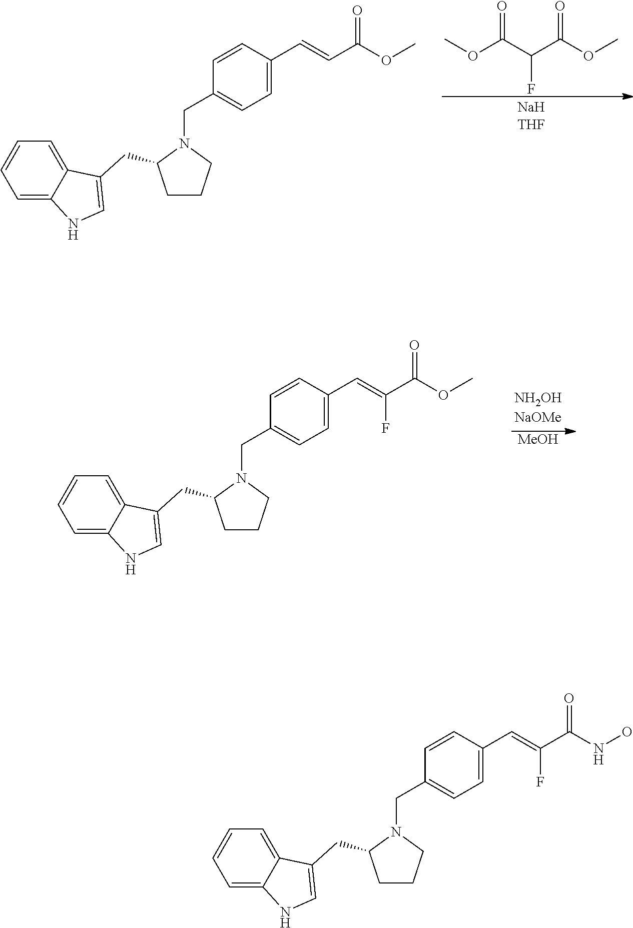

### Step a: Preparation of (Z)-2-fluoro-3-{4-[(R)-2-(1H-indol-3-ylmethyl)-pyrrolidin-1-ylmethyl]-phenyl}-acrylic acid methyl ester

Following procedures analogous to those described in Example 1, Steps (a)-(e), (E)-3-{4-[(R)-2-(1H-indol-3-ylmethyl)-pyrrolidin-1-ylmethyl]-phenyl}-acrylic acid methyl ester is prepared as a white solid. LC-MS: 374.

To a mixture of sodium hydride (38 mg, 0.96 mmol) and tetrahydrofuran (1 mL) at 0° C. under a nitrogen atmosphere is added dimethyl 2-fluoromalonate (144 mg, 0.96 mmol) in dried tetrahydrofuran (1 mL) and the mixture is stirred for 30 minutes. (E)-3-{4-[(R)-2-(1H-Indol-3-ylmethyl)-pyrrolidin-1-ylmethyl]-phenyl}-acrylic acid methyl ester (300 mg, 0.8 mmol) is added and the resulting reaction is refluxed for 12 hours under nitrogen atmosphere. The reaction is quenched with ice water and extracted three times with diethyl ether (15 mL). The combined organic layers are washed with brine (20 mL), dried over magnesium sulfate and concentrated under reduced pressure. The crude product is purified by a silica gel column chromatography (12-100% ethyl acetate/heptanes) to provide (Z)-2-fluoro-3-{4-[(R)-2-(1H-indol-3-ylmethyl)-pyrrolidin-1-ylmethyl]-phenyl}-acrylic acid methyl ester (90 mg, 28.6%) as a light yellow oil. LC-MS: 393.

### Step b: Preparation of (Z)-2-fluoro-N-hydroxy-3-{4-[(R)-2-(1H-indol-3-ylmethyl)-pyrrolidin-1-ylmethyl]-phenyl}-acrylamide

Following procedures analogous to those described in Example 1, Step (1), (Z)-2-fluoro-N-hydroxy-3-{4-[(R)-2-(1H-indol-3-ylmethyl)-pyrrolidin-1-ylmethyl]-phenyl}-acrylamide (24 mg, 0.061 mmol, 26.5% yield) is prepared as a white solid. HRMS: 394.1937.

### Example 5

### Preparation of (E)-N-hydroxy-3-(4-{(R)-1-[(R)-2-(2-methyl-1H-indol-3-ylmethyl)-pyrrolidin-1-yl]-ethyl}-phenyl)-acrylamide (17) and (E)-N-hydroxy-3-(4-{(S)-1-[(R)-2-(2-methyl-1H-indol-3-ylmethyl)-pyrrolidin-1-yl]-ethyl}-phenyl)-acrylamide (14)

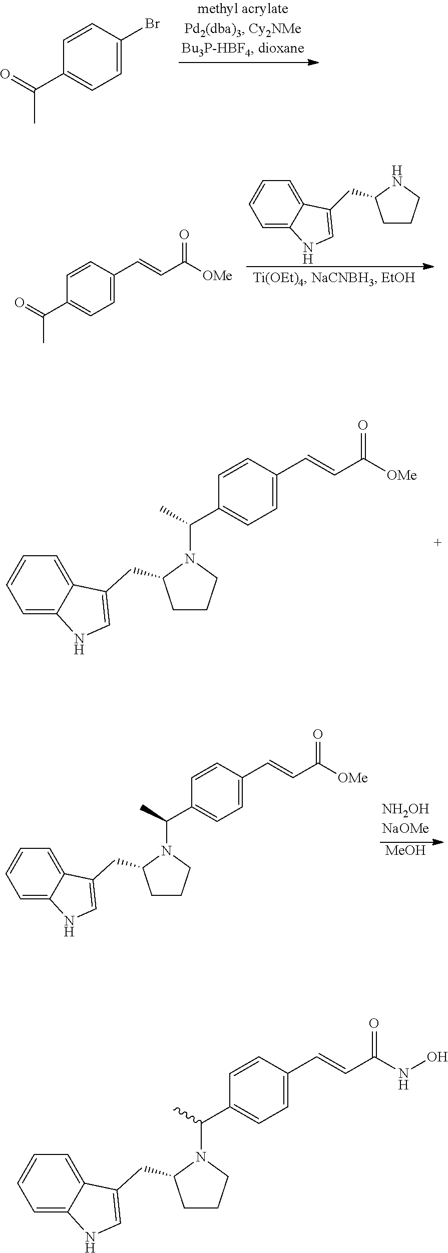

### Step a: Preparation of (E)-3-(4-acetyl-phenyl)-acrylic acid methyl ester

A mixture of 1-(4-bromo-phenyl)-ethanone (48.0 g, 241 mmol), methyl acrylate (43.0 mL, 48 mmol), N-methyldicyclohexylamine (61 mL, 288 mmol), tri-(tert-butyl)phosphine-tetrafluoroborate (2.78 g, 9.6 mmol) and Pd₂(dba)₃ (2.2 g, 2.4 mmol) in 1,4-dioxane (160 mL) is flushed with N₂ and heated at 100° C. for 2 hours. The reaction mixture is cooled to room temperature and filtered through a Celite pad, which is rinsed with ethyl acetate. The filtrate and the washes are combined and concentrated. The residue is purified by a silica gel column chromatography (ethyl acetate/heptane) to give (E)-3-(4-acetyl-phenyl)-acrylic acid methyl ester (17 g, 34% yield). LCMS 205.

### Step b: Preparation of (E)-3-(4-{(R)-1-[(R)-2-(1H-indol-3-ylmethyl)-pyrrolidin-1-yl]-ethyl}-phenyl)-acrylic acid methyl ester and (E)-3-(4-{(S)-1-[(R)-2-(1H-indol-3-ylmethyl)-pyrrolidin-1-yl]-ethyl}-phenyl)-acrylic acid methyl ester

To a solution of (E)-3-(4-acetyl-phenyl)-acrylic acid methyl ester (1.02 g, 5.0 mmol) in ethanol (25 mL) is added 3-(R)-1-pyrrolidin-2-ylmethyl-1H-indole (1.07 g, 5.0 mmol) at room temperature. The resulting solution is stirred for 30 minutes, titanium ethoxide (1.25 g, 5.5 mmol) is added, and the resulting mixture is stirred for 30 minutes. Sodium cyanoborohydride (630 mg, 10 mmol) is added and the resulting mixture is stirred at room temperature for 12 hours and concentrated under reduced pressure. The residue is diluted with ethyl acetate (30 mL) and a saturated solution of sodium bicarbonate (20 mL) is added. The resulting mixture is extracted three times with 30 mL of ethyl acetate and the organic layers are combined, washed with a saturated solution of sodium chloride (20 mL), dried over magnesium sulfate, filtered, and concentrated in vacuo. The residue is purified by a silica gel column chromatography (20-100% ethyl acetate/heptanes) to provide (E)-3-(4-{(R)-1-[(R)-2-(1H-indol-3-ylmethyl)-pyrrolidin-1-yl]-ethyl}-phenyl)-acrylic acid methyl ester and (E)-3-(4-{(S)-1-[(R)-2-(1H-indol-3-ylmethyl)-pyrrolidin-1-yl]-ethyl}-phenyl)-acrylic acid methyl ester (1.52 g, 75% combined yield, stereochemistry at benzyl position is not identified) as white powders.

Following procedures analogous to those described in Example 1, Step (I), (E)-N-hydroxy-3-(4-{(R)-1-[(R)-2-(2-methyl-1H-indol-3-ylmethyl)-pyrrolidin-1-yl]-ethyl}-phenyl)-acrylamide (15, HRMS: 404.2338) and (E)-N-hydroxy-3-(4-{(S)-1-[(R)-2-(2-methyl-1H-indol-3-ylmethyl)-pyrrolidin-1-yl]-ethyl}-phenyl)-acrylamide (14, HRMS: 404.2346) are prepared.

Following procedures analogous to those described in Example 1, Steps (e) and (f), (E)-N-hydroxy-3-{4-[1-((S)-2-pyrrolidin-1-ylmethyl-pyrrolidin-1-yl)-ethyl]-phenyl}-acrylamide (16) is prepared. HRMS: 344.2341 (M+1).

### Example 6

### Preparation of (E)-3-{4-[(S)-2-(2,3-dihydro-indol-1-ylmethyl)-pyrrolidin-1-ylmethyl]-phenyl}-N-hydroxy-acrylamide (19)

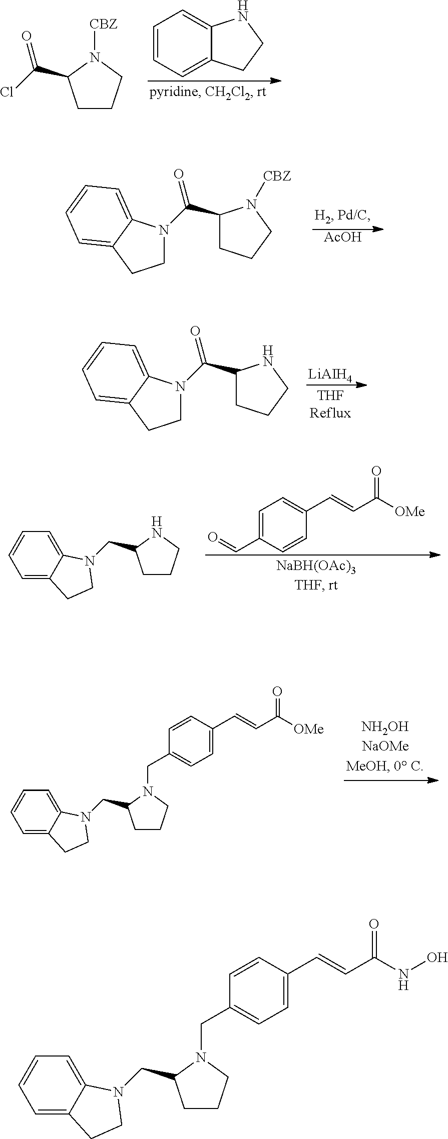

### Step a: Preparation of (S)-2-(2,3-dihydro-indole-1-carbonyl)-pyrrolidine-1-carboxylic acid benzyl ester

To a solution of (S)-2-chlorocarbonyl-pyrrolidine-1-carboxylic acid benzyl ester (2.68 g, 10 mmol) in dichloromethane is added a solution of indoline (1.20 g, 10.0 mmol) and pyridine (1.4 mL, 17.1 mmol) in dichloromethane (15 mL) at 0° C. The mixture is stirred for 4 hours at room temperature and washed with water, a saturated aqueous sodium bicarbonate solution, hydrochloric acid (1 N) and brine. The solvent is removed under reduced pressure to provide the crude product as a tan solid (3.0 g, 86%).

### Step b: Preparation of (2,3-dihydro-indol-1-yl)-(S)-pyrrolidin-2-yl-methanone

To a flask charged with palladium on active carbon (930 mg) is added solution of (S)-2-(2,3-dihydro-indole-1-carbonyl)-pyrrolidine-1-carboxylic acid benzyl ester (3.0 g, 8.56 mmol) in acetic acid (36 mL). The mixture is degassed and refilled with hydrogen via a balloon and this process is repeated five times. The mixture is stirred overnight under hydrogen atmosphere and the solid is filtered via a pad of Celite. The filtrate is diluted with dichloromethane (40 mL) and washed with a saturated sodium bicarbonate solution (100 mL). The aqueous washes are combined and extracted three times with dichloromethane (120 mL). The combined organic layers are washed with a saturated sodium chloride solution, dried over magnesium sulfate, filtered, and concentrated under reduced pressure to provide (2,3-dihydro-indol-1-yl)-(S)-pyrrolidin-2-yl-methanone (441 mg, 24%) as a dark sticky oil.

### Step c: Preparation of 1-(S)-1-pyrrolidin-2-ylmethyl-2,3-dihydro-1H-indole

To a solution of lithium aluminum hydride (106 mg, 2.78 mmol) in tetrahydrofuran (3.6 mL) is add a solution of (2,3-dihydro-indol-1-yl)-(S)-pyrrolidin-2-yl-methanone (200 mg, 0.93 mmol) in tetrahydrofuran (3.1 mL) at 0° C. The reaction mixture is refluxed for 12 hours and cooled to 0° C. While stirring vigorously, a mixture of sodium sulfate pentahydrate (1.0 g) and Celite (300 mg) is added in portions. The mixture is filtered and the solid rinsed with methanol and ethyl acetate. The filtrate and washes are concentrated under reduced pressure to provide 1-(S)-1-pyrrolidin-2-ylmethyl-2,3-dihydro-1H-indole (98 mg, 52%) as an amber liquid.

### Step d: Preparation of (E)-3-{4-[(S)-2-(2,3-dihydro-indol-1-ylmethyl)-pyrrolidin-1-ylmethyl]-phenyl}-N-hydroxy-acrylamide (19)

Following procedures analogous to those described in Example 1, Steps (e) and (f), (E)-3-{4-[(S)-2-(2,3-dihydro-indol-1-ylmethyl)-pyrrolidin-1-ylmethyl]-phenyl}-N-hydroxy-acrylamide (21, 8% yield) is prepared as a white solid. FIR-MS: 378.2181.

Following procedures analogous to those described in Examples 6, Steps (a) and (b), and 1, Steps (e) and (1), (E)-3-{4-[(R)-2-(2,3-dihydro-indole-1-carbonyl)-pyrrolidin-1-ylmethyl]-phenyl}-N-hydroxy-acrylamide (17, HR-MS: 392.1974) and (E)-3-{4-[(S)-2-(2,3-dihydro-indole-1-carbonyl)-pyrrolidin-1-ylmethyl]-phenyl}-N-hydroxy-acrylamide (18, HRMS: 392.1961) are prepared.

### Example 7

### Preparation of (E)-N-hydroxy-3-{4-[(2S,4S)-4-hydroxy-2-(2-methyl-1H-indol-3-ylmethyl)-pyrrolidin-1-ylmethyl]-phenyl}-acrylamide (21)

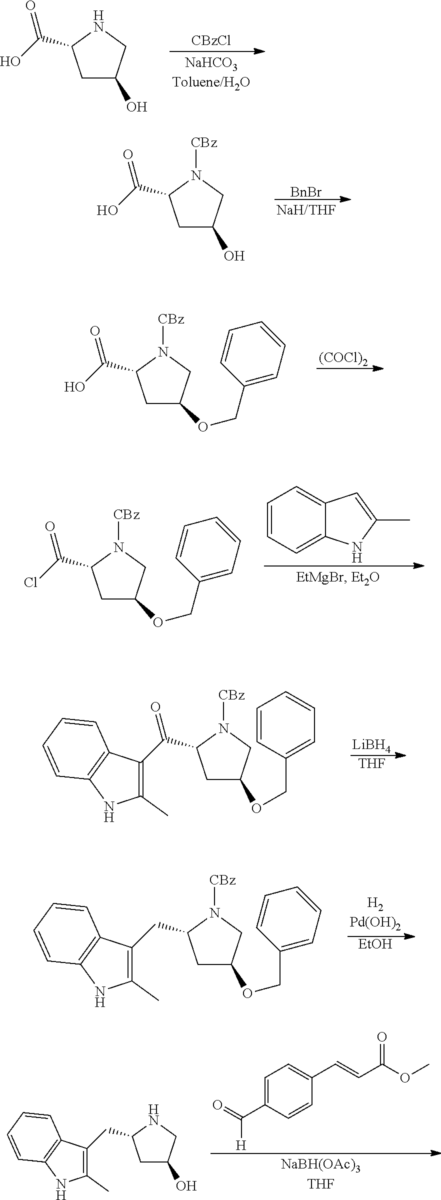

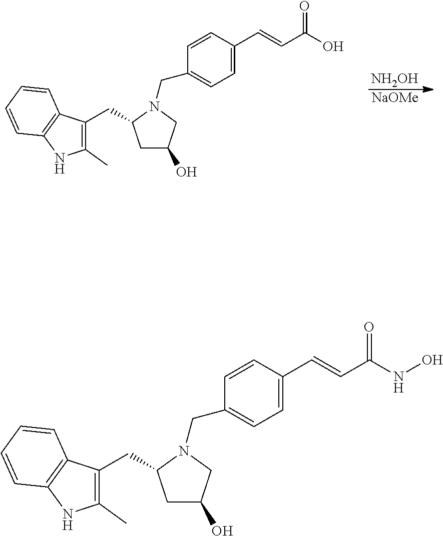

### Step a: Preparation of (2R,4R)-4-hydroxy-pyrrolidine-1,2-dicarboxylic acid 1-benzyl ester

To a solution of cis-4-hydroxy-L-proline (10.0 g, 76.3 mmol) and sodium bicarbonate ((16.0 g, 190 mmol) in water (165 mL) is added a solution of benzyl chloroformate (12.5 mL, 15.0 g, 87.7 mmol) in toluene (40 mL) over a period of 15 minutes and the resulting solution is stirred at room temperature for 16 hours. The two phases are separated and the aqueous phase is extracted with ether (4×50 mL), cooled in an ice bath, acidified to pH 2 with concentrated hydrochloric acid, and extracted with ethyl acetate (5×50 mL). The organic extracts are combined, dried over magnesium sulfate, and concentrated to provide (2R,4R)-4-hydroxy-pyrrolidine-1,2-dicarboxylic acid 1-benzyl ester (18.9 g, 93%) as a viscous oil.

### Step b: Preparation of (2R,4R)-4-benzyloxy-pyrrolidine-1,2-dicarboxylic acid 1-benzyl ester

A solution of (2R,4R)-4-hydroxy-pyrrolidine-1,2-dicarboxylic acid 1-benzyl ester (7.44 g, 28 mmol) in anhydrous tetrahydrofuran (270 mL) is treated with sodium hydride (60% in oil, 2.36 g, 59 mmol). The reaction mixture is stirred at room temperature for 1 hour and treated with benzyl bromide (9.58 g, 56 mmol). The resulting mixture is heated under reflux for 5 hours, cooled to room temperature, quenched with ice water, and extracted with heptane. The aqueous solution is acidified with 1N hydrochloride acid and extracted three times with ethyl acetate (300 mL). The organic layers are combined, washed with a saturated sodium chloride solution, dried over magnesium sulfate, and concentrated under reduced pressure to provide (2R,4R)-4-benzyloxy-pyrrolidine-1,2-dicarboxylic acid 1-benzyl ester (8.1 g, 81%) as a colorless oil. LC-MS: 356.

Following procedures analogous to those described in Example 1 (in Example 1, Step (d), one equivalent of palladium hydroxide on carbon is used to remove benzyl protection group), the following compounds are prepared:

| Cpd | Name | MS |
|---|---|---|
| 20 | (E)-N-Hydroxy-3-{4-[(2S,4R)-4-hydroxy-2-(2-methyl-1H-indol-3-ylmethyl)- | 406.2137 |
| | pyrrolidin-1-ylmethyl]-phenyl}-acrylamide | |
| 21 | (E)-N-Hydroxy-3-{4-[(2S,4S)-4-hydroxy-2-(2-methyl-1H-indol-3-ylmethyl)- | 406.2131 |
| | pyrrolidin-1-ylmethyl]-phenyl}-acrylamide | |
| 22 | (E)-N-Hydroxy-3-{4-[(2S,4S)-4-hydroxy-2-(1H-indol-3-ylmethyl)- | 392.1791 |
| | pyrrolidin-1-ylmethyl]-phenyl}-acrylamide | |
| 23 | (E)-N-Hydroxy-3-{4-[(2S,4R)-4-hydroxy-2-(1H-indol-3-ylmethyl)- | 392.1 |
| | pyrrolidin-1-ylmethyl]-phenyl}-acrylamide | |

Following procedures analogous to those described in Examples 7, Steps (a) and (b) (benzyl bromide is replaced with methyl iodide), and 1, Steps (a)-(d), the following compounds are prepared.

| Cpd | Name | MS |
|---|---|---|
| 24 | (E)-N-hydroxy-3-{4-[(2S,4R)-4-methoxy-2-(2-methyl-1H-indol-3-ylmethyl)- | 420.2300 |
| | pyrrolidin-1-ylmethyl]-phenyl}-acrylamide | |
| 25 | (E)-N-hydroxy-3-{4-[(2S,4S)-4-methoxy-2-(2-methyl-1H-indol-3-ylmethyl)- | 420.2280 |
| | pyrrolidin-1-ylmethyl]-phenyl}-acrylamide | |

### Example 8

### Preparation of (E)-N-hydroxy-3-{6-[(R)-2-(2-methyl-1,1-indol-3-ylmethyl)-pyrrolidin-1-ylmethyl]-pyridin-3-yl}-acrylamide (26)

### Step a: Preparation of (E)-3-(6-formyl-pyridin-3-yl)-acrylic acid methyl ester

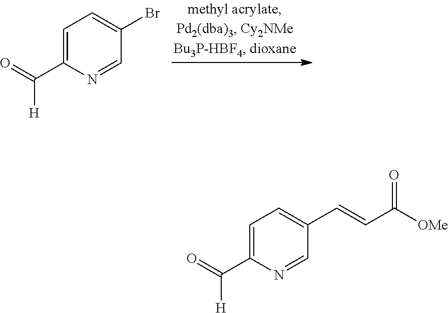 [SMILES: O=Cc1ccc(Br)cn1 ; COC(=O)/C=C/c1ccc(C=O)nc1]

A mixture of 5-bromo-pyridine-2-carbaldehyde (1 g, 5.4 mmol), N-methyldicyclohexylamine (1.37 mL, 6.45 mmol), tri-(tert-butyl)phosphoine-tetrafluoroborate (62.4 mg, 0.215 mmol) and Pd₂(dba)₃ (49.2 mg, 0.054 mmol) in 1,4-dioxane (5 mL) is charged in a sealed dry microwave vial and stirred for 30 minutes under N₂. Methyl acrylate (2.2 mL, 24.6 mmol) is added and the resulting reaction is heated at 100° C. for 30 minutes in microwave reactor, cooled to room temperature, and filtered through a Celite pad, which is rinsed with ethyl acetate. The filtrate and washes are combined and concentrated and the residue is purified by a silica gel column chromatography (ethyl acetate/heptane) to give (E)-3-(6-formyl-pyridin-3-yl)-acrylic acid methyl ester (0.768 g, 75% yield). LCMS: 192.2.

Following procedures analogous to those described in Example 1, (E)-N-hydroxy-3-{6-[(R)-2-(2-methyl-1H-indol-3-ylmethyl)-pyrrolidin-1-ylmethyl]-pyridin-3-yl}-acrylamide (26) is prepared. LCMS: 391.2117.

Following procedures analogous to those described in Examples 7, Steps (a) and (b) (benzyl bromide is replaced with methyl iodide), and 1, Steps (a)-(1), (E)-N-hydroxy-3-{6-[(2S,4S)-4-hydroxy-2-(2-methyl-1H-indol-3-ylmethyl)-pyrrolidin-1-ylmethyl]-pyridin-3-yl}-acrylamide (28, LCMS: 407.2068) and (E)-N-Hydroxy-3-{6-[(2S,4R)-4-hydroxy-2-(2-methyl-1H-indol-3-ylmethyl)-pyrrolidin-1-ylmethyl]-pyridin-3-yl}-acrylamide (27, HRMS: 407.2083) are prepared.

### Example 9

### Preparation of (E)-3-{4-[(2S,4S)-4-amino-2-(2-methyl-1H-indol-3-ylmethyl)-pyrrolidin-1-ylmethyl]-phenyl}-N-hydroxy-acrylamide (29)

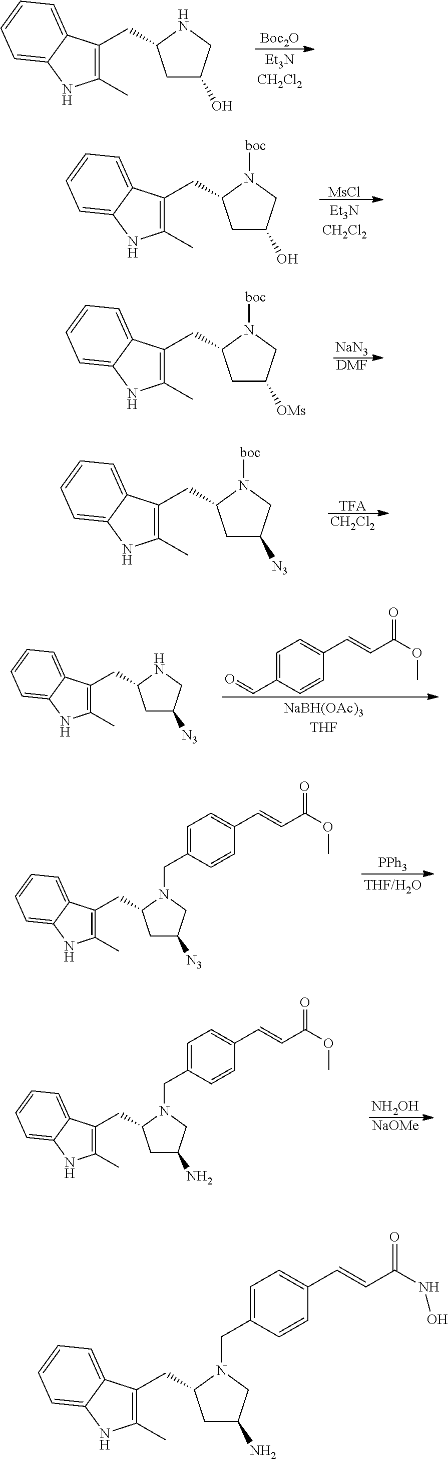

### Step a: Preparation of (2S,4R)-4-hydroxy-2-(2-methyl-1H-indol-3-ylmethyl)-pyrrolidine-1-carboxylic acid tert-butyl ester

Di-tert-butyl carbonate (1.14 g, 5.2 mmol) is added to a stirred solution of (3R,5S)-5-(2-methyl-1H-indol-3-ylmethyl)-pyrrolidin-3-ol (1.0 g, 3.0 mmol) and triethyl amine (1.21 mL, 8.7 mmol) in dichloromethane (8 mL) at 0° C. After 20 minutes, the cold bath is removed and stirring is continued for 12 h. The reaction mixture is diluted with dichloromethane (20 mL), washed with water, a saturated aqueous sodium bicarbonate solution, and a saturated solution of sodium chloride, dried over sodium sulfate, and concentrated. The crude product is purified by a silica gel column chromatography (0-10%, methanol/dichloromethane) to give (2S,4R)-4-hydroxy-2-(2-methyl-1H-indol-3-ylmethyl)-pyrrolidine-1-carboxylic acid tert-butyl ester as a light brown solid (734 mg, 74% yield). LC-MS: 329.

### Step b: Preparation of (2S,4R)-4-methanesulfonyloxy-2-(2-methyl-1H-indol-3-ylmethyl)-pyrrolidine-1-carboxylic acid tert-butyl ester

To a solution of (2S,4R)-4-hydroxy-2-(2-methyl-1H-indol-3-ylmethyl)-pyrrolidine-1-carboxylic acid tert-butyl ester (734 mg, 2.2 mmol) and triethylamine (620 uL, 4.4 mmol) in dichloromethane (10 mL) is added methanesulfonyl chloride (260 uL, 3.3 mmol) at 0° C. The mixture is stirred for 3 hours and poured into water, and the resulting mixture is extracted with ethyl acetate. The organic layers are combined, washed with a saturated solution of sodium chloride, dried over magnesium sulfate, and concentrated in vacuo to afford (2S,4R)-4-methanesulfonyloxy-2-(2-methyl-1H-indol-3-ylmethyl)-pyrrolidine-1-carboxylic acid tert-butyl ester (915 mg, quantitative yield) as a light brown solid. The crude product is used in the subsequent step without further purification. LC-MS: 409.

### Step c: Preparation of (2S,4S)-4-azido-2-(2-methyl-1H-indol-3-ylmethyl)-pyrrolidine-1-carboxylic acid tert-butyl ester

Sodium azide (730 mg, 11.2 mmol) is added to a stirred solution of (2S,4R)-4-methanesulfonyloxy-2-(2-methyl-1H-indol-3-ylmethyl)-pyrrolidine-1-carboxylic acid tert-butyl ester (915 mg, 2.24 mmol) in dry N,N-dimethylformamide (11 mL) at room temperature. The reaction mixture is stirred at 90° C. for 4 hours and concentrated. The residue is partitioned between a mixture of saturated sodium bicarbonate solution (15 mL) and ethyl acetate (15 mL). The combined organic phases are washed with brine (20 mL), dried over sodium sulfate, filtered, and concentrated. The residue is purified by a silica gel column chromatography (12-100%, ethyl acetate/heptanes) to give (2S,4S)-4-azido-2-(2-methyl-1H-indol-3-ylmethyl)-pyrrolidine-1-carboxylic acid tert-butyl ester (450 mg, 56% yield) as a white solid. LC-MS: 356.3.

### Step d: Preparation of 3-((2S,4S)-4-azido-pyrrolidin-2-ylmethyl)-2-methyl-1H-indole

(2S,4S)-4-Azido-2-(2-methyl-1H-indol-3-ylmethyl)-pyrrolidine-1-carboxylic acid tert-butyl ester (300 mg, 0.85 mmol) is dissolved in 3 mL, of dichloromethane and the solution is cooled to −78° C. Trifluoroacetic acid (3 mL) is added, and the solution is warmed to room temperature slowly and stirred for 1 hour. The reaction mixture is concentrated and diluted with dichloromethane, and the resulting mixture is washed with a saturated sodium bicarbonate solution, a saturated solution of sodium chloride, dried over sodium sulfate, filtered, and concentrated. The crude product is used in the subsequent step without further purification. LC-MS 256.3.

### Step e: Preparation of (E)-3-{4-[(2S,4S)-4-azido-2-(2-methyl-1H-indol-3-ylmethyl)-pyrrolidin-1-ylmethyl]-phenyl}-acrylic acid methyl ester

Following procedures analogous to those described in Example 1, Step (e), (E)-3-{4-[(2S,4S)-4-azido-2-(2-methyl-1H-indol-3-ylmethyl)-pyrrolidin-1-ylmethyl]-phenyl}-acrylic acid methyl ester is prepared and carried out in the subsequent step with some minor impurities. LC-MS: 430.3.

### Step f: Preparation of (E)-3-{4-[(2S,4S)-4-amino-2-(2-methyl-1H-indol-3-ylmethyl)-pyrrolidin-1-ylmethyl]-phenyl}-acrylic acid methyl ester

To a stirred solution of (E)-3-{4-[(2S,4S)-4-azido-2-(2-methyl-1H-indol-3-ylmethyl)-pyrrolidin-1-ylmethyl]-phenyl}-acrylic acid methyl ester (0.85 mmol) in tetrahydrofuran (7 mL) is added triphenylphosphine (445 mg, 1.7 mmol) at 0° C. The reaction mixture is stirred for 30 minutes and ammonium hydroxide/water solution (2/0.4 mL) is added. The resulting solution is stirred at room temperature overnight and 1N hydrochloride acid solution is added. The mixture is washed with diethyl ether, basified to pH>10, and extracted ethyl acetate. The organic layers are combined and concentration to give (E)-3-{4-[(2S,4S)-4-amino-2-(2-methyl-1H-indol-3-ylmethyl)-pyrrolidin-1-ylmethyl]-phenyl}-acrylic acid methyl ester (80 mg), which is used in the subsequent step without further purification. LC-MS: 404.3.

Following procedures analogous to those described in Example 1, Step (f), (E)-3-{4-[(2S,4S)-4-amino-2-(2-methyl-1H-indol-3-ylmethyl)-pyrrolidin-1-ylmethyl]-phenyl}-N-hydroxy-acrylamide (31) is prepared after HPLC purification. HRMS: 405.2289.

### Example 10

### Preparation of (E)-3-{4-[(S)-4-fluoro-2-(2-methyl-1H-indol-3-ylmethyl)-pyrrolidin-1-ylmethyl]-phenyl}-N-hydroxy-acrylamide (30)

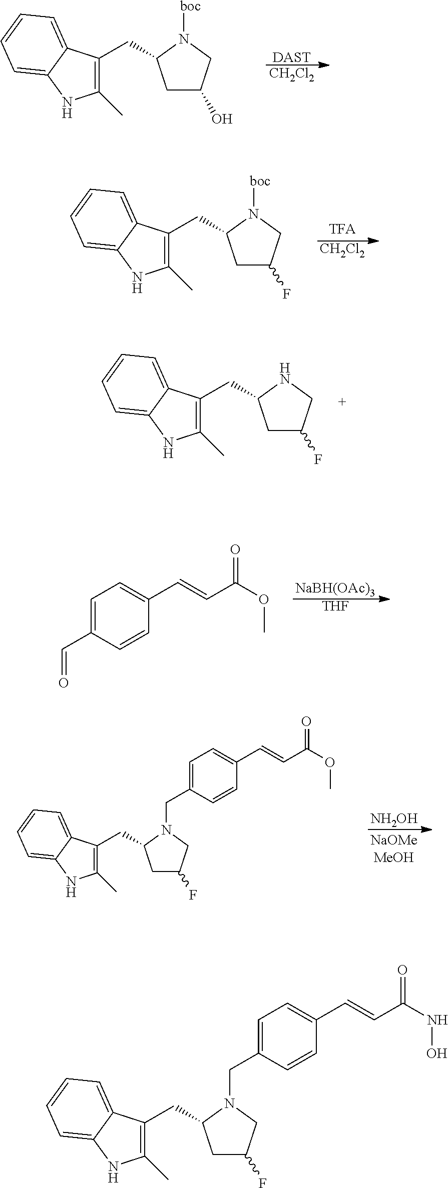

### Step a: Preparation of (S)-4-fluoro-2-(2-methyl-1H-indol-3-ylmethyl)-pyrrolidine-1-carboxylic acid tert-butyl ester

To a solution of (diethylamino)sulfur trifluoride (113 uL, 0.86 mmol) in ethyl acetate (1.2 mL) at −78° C. is added a solution of (2S,4R)-4-hydroxy-2-(2-methyl-1H-indol-3-ylmethyl)-pyrrolidine-1-carboxylic acid tert-butyl ester (284 mg, 0.86 mmol) in ethyl acetate (0.6 mL). The reaction mixture is stirred at −78° C. for 2 hours, slowly warmed to room temperature, and stirred for 10 hours. The reaction mixture is quenched with a saturated sodium bicarbonate solution and a small portion of magnesium sulfate added. The solution is separated, the aqueous layer is extracted with ethyl acetate (15 mL), and the organic layers are combined, washed with brine, dried over sodium sulfate, filtered, and concentrated. The residue is purified via a silica gel column chromatography (0-10%, methanol/dichloromethane) to give (S)-4-fluoro-2-(2-methyl-1H-indol-3-ylmethyl)-pyrrolidine-1-carboxylic acid tert-butyl ester (53 mg, 18% yield). LC-MS: 331.

### Step b: Preparation of 3-((S)-4-fluoro-pyrrolidin-2-ylmethyl)-2-methyl-1H-indole

(S)-4-Fluoro-2-(2-methyl-1H-indol-3-ylmethyl)-pyrrolidine-1-carboxylic acid tert-butyl ester (100 mg, 0.3 mmol) is dissolved in 1 mL of dichloromethane and the solution is cooled to −78° C. Trifluoroacetic acid (1 mL) is added and the solution is warmed to room temperature slowly and stirred for 1 h at room temperature. The reaction mixture is concentrated and the residue is diluted with dichloromethane. The resulting solution is washed with a saturated sodium bicarbonate solution, a saturated solution of sodium chloride, dried over sodium sulfate, filtered and concentrated. The crude product is used in the subsequent step without further purification. LC-MS 233.1.

### Step c: Preparation of (E)-3-{4-[(S)-4-fluoro-2-(2-methyl-1H-indol-3-ylmethyl)-pyrrolidin-1-ylmethyl]-phenyl}-acrylic acid methyl ester

Following procedures analogous to those described in Example 1, Step (e), (E)-3-{4-[(S)-4-fluoro-2-(2-methyl-1H-indol-3-ylmethyl)-pyrrolidin-1-ylmethyl]-phenyl}-acrylic acid methyl ester is prepared with some minor impurities. The mixture is used in the subsequent reaction without further purification. LC-MS: 407.3.

Following procedures analogous to those described in Example 1, Step (f), (E)-3-{4-[(S)-4-fluoro-2-(2-methyl-1H-indol-3-ylmethyl)-pyrrolidin-1-ylmethyl]-phenyl}-N-hydroxy-acrylamide (31) is prepared after HPLC purification. HRMS: 408.2069.

### Example 11

### Preparation of (E)-N-hydroxy-3-{4-[(2S,4S)-4-hydroxy-2-(1,3,5-trimethyl-1H-pyrazol-4-ylmethyl)-pyrrolidin-1-ylmethyl]-phenyl}-acrylamide (31)

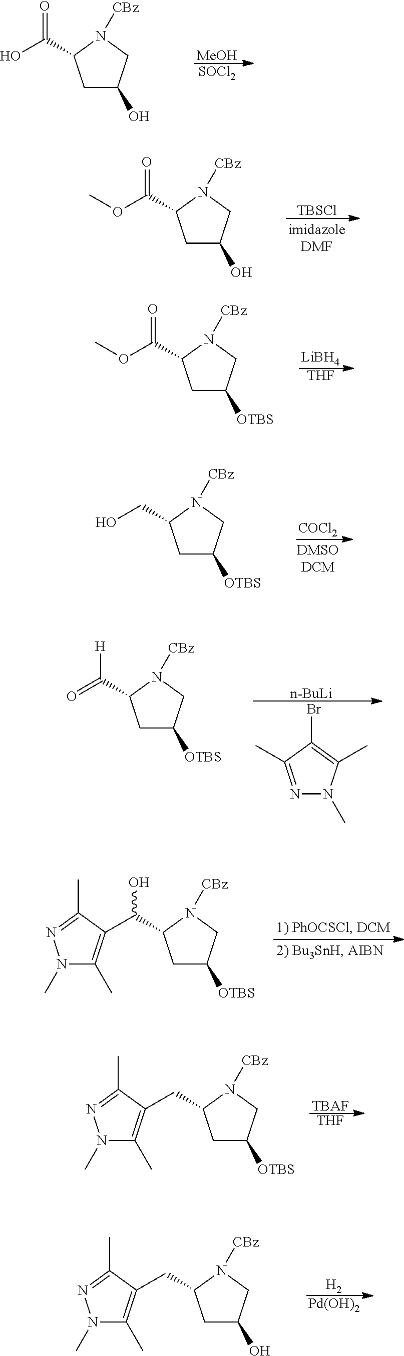

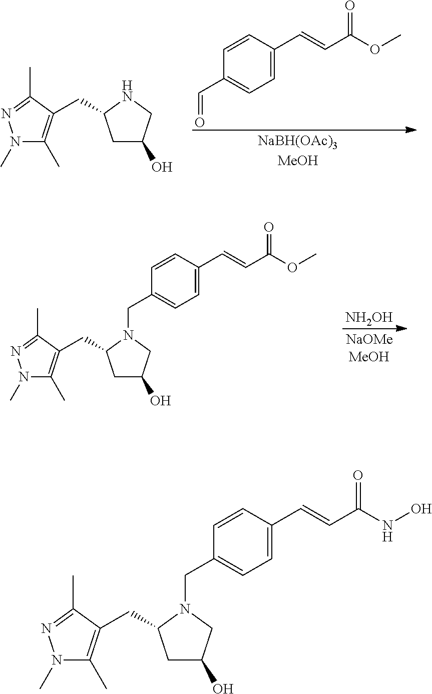

### Step a: Preparation of (2R,4S)-4-hydroxy-pyrrolidine-1,2-dicarboxylic acid 1-benzyl ester 2-methyl ester

A solution of (2R,4S)-4-hydroxy-pyrrolidine-1,2-dicarboxylic acid 1-benzyl ester (9.36 g, 0.034 mol) in methanol (60 mL) is added to a solution of thionyl chloride (7.8 mL, 98 mmol) in methanol (100 mL) at 0° C. and the resulting mixture is stirred at room temperature for 12 hours. The reaction mixture is concentrated under reduced pressure to give (2R,4S)-4-hydroxy-pyrrolidine-1,2-dicarboxylic acid 1-benzyl ester 2-methyl ester (in quantitative yield) as a yellow oil. The product is used in the subsequent reaction without further purification. LC-MS: 280.

### Step b: Preparation of (2R,4S)-4-(tert-butyl-dimethyl-silanyloxy)-pyrrolidine-1,2-dicarboxylic acid 1-benzyl ester 2-methyl ester

Imidazol (5.37 g, 79 mmol), N,N-diisopropylethylamine (82 mL, 54 mmol), and tert-butyl-diphenylsilyl chloride (8.10 g, 54 mmol) are added to a stirred solution of (2R,4S)-4-hydroxy-pyrrolidine-1,2-dicarboxylic acid 1-benzyl ester 2-methyl ester (10.04 g, 36 mmol) in dry N,N-dimethylformamide (80 mL). The reaction mixture is stirred overnight and concentrated, and the residue is partitioned between a mixture of saturated sodium bicarbonate solution (200 mL) and ethyl acetate (200 mL). The organic phase is washed with a saturated sodium chloride solution (50 mL), dried over magnesium sulfate, filtered, and concentrated. The residue is purified via a silica gel column chromatography (12-100% ethyl acetate/heptanes) to give (2R,4S)-4-(tert-butyl-dimethyl-silanyloxy)-pyrrolidine-1,2-dicarboxylic acid 1-benzyl ester 2-methyl ester (8.49 g, 60% yield) as a colorless oil. LC-MS: 394.

### Step c: Preparation of (2R,4S)-4-(tert-butyl-dimethyl-silanyloxy)-2-hydroxymethyl-pyrrolidine-1-carboxylic acid benzyl ester

Lithium borohydride (14.0 mL, 2.0 M in tetrahydrofuran) is slowly added to a stirred solution of (2R,4S)-4-(tert-butyl-dimethyl-silanyloxy)-pyrrolidine-1,2-dicarboxylic acid 1-benzyl ester 2-methyl ester (8.49 g, 21.6 mmol) in dry tetrahydrofuran (60 mL) at 0° C. The reaction mixture is stirred 12 h and cooled to 0° C. Water (100 mL) is added followed by the slow addition of 1 N hydrochloride acid solution (50 mL). The acidic solution is extracted three times with ethyl acetate (300 mL). The combined organic phases are washed with a saturated sodium chloride solution (100 mL), a saturated sodium bicarbonate solution (100 mL), a saturated sodium chloride solution (100 mL), dried over magnesium sulfate, and concentrated. The residue is purified by a silica gel column chromatography (12-100% ethyl acetate/heptanes) to give (2R,4S)-4-(tert-butyl-dimethyl-silanyloxy)-2-hydroxymethyl-pyrrolidine-1-carboxylic acid benzyl ester (7.12 g, 90%) as a colorless oil.

### Step d: Preparation of (2R,4S)-4-(tert-butyl-dimethyl-silanyloxy)-2-formyl-pyrrolidine-1-carboxylic acid benzyl ester

To a solution of dimethyl sulfoxide (1.1 mL, 16.0 mmol) in dichloromethane (50 mL) is added oxalyl chloride (670 uL, 8.0 mmol) at −78° C. and the resulting mixture is stirred for 15 minutes. (2R,4S)-4-(Tert-butyl-dimethyl-silanyloxy)-2-hydroxymethyl-pyrrolidine-1-carboxylic acid benzyl ester (1.46 g, 4.0 mmol) is added slowly and the resulting mixture is stirred at −78° C. for 1 hour. Triethylamine (3.3 mL, 24.0 mmol) is added, and the solution is allowed to warm slowly to room temperature, quenched with a saturated sodium bicarbonate solution, washed with a saturated sodium chloride solution, dried over magnesium sulfate, filtered, and concentrated under reduced pressure. The crude material is used in the subsequent step immediately without further purification.

### Step e: Preparation of (2R,4S)-4-(tert-butyl-dimethyl-silanyloxy)-2-[hydroxy-(1,3,5-trimethyl-1H-pyrazol-4-yl)-methyl]-pyrrolidine-1-carboxylic acid benzyl ester

To a solution of 4-bromo-1,3,5-trimethyl-1H-pyrazole (950 mg, 5.0 mmol) in tetrahydrofuran (25 mL) is added n-butyllithium (2.1 mL, 2.5 M in hexane) at −78° C. and the resulting solution is stirred for 30 minutes. A solution of (2R,4S)-4-(tert-butyl-dimethyl-silanyloxy)-2-formyl-pyrrolidine-1-carboxylic acid benzyl ester (4 mmol) in tetrahydrofuran (5 mL) is added and the reaction mixture is slowly warmed to 0° C. over 30 minutes, stirred for 30 minutes, and quenched with ice water (40 mL). The organic layer is separated and the aqueous layer is extracted three times with ethyl acetate (60 mL). The combined organic phases are washed with a saturated sodium chloride solution, dried over magnesium sulfate, filtered and concentrated. The crude material is purified with a silica gel column chromatography (40-100% ethyl acetate/heptanes) to provide (2R,4S)-4-(tert-butyl-dimethyl-silanyloxy)-2-[hydroxy-(1,3,5-trimethyl-1H-pyrazol-4-yl)-methyl]-pyrrolidine-1-carboxylic acid benzyl ester (280 mg, 12%) as a light yellow oil. LC-MS: 474.1 (M+1).

### Step f: Preparation of (2S,4S)-4-(tert-butyl-dimethyl-silanyloxy)-2-(1,3,5-trimethyl-1H-pyrazol-4-ylmethyl)-pyrrolidine-1-carboxylic acid benzyl ester

To a solution of (2R,4S)-4-(tert-butyl-dimethyl-silanyloxy)-2-[hydroxy-(1,3,5-trimethyl-1H-pyrazol-4-yl)-methyl]-pyrrolidine-1-carboxylic acid benzyl ester (280 mg, 0.59 mmol) in dichloromethane (3 mL) at 0° C. is added pyridine (157 uL, 1.95 mmol) and phenyl chlorothionoformate (107 mg, 0.62 mmol). The mixture is stirred at 0° C. for 30 minutes and at room temperature for 8 hours. The solution is quenched with a saturated sodium bicarbonate solution and the aqueous phase is extracted three times with ethyl acetate (15 mL). The combined organic phases are washed with a saturated sodium chloride solution, dried with magnesium sulfate, filtered, and concentrated. The residue is purified with a silica gel column chromatography to give (2R,4S)-4-(tert-butyl-dimethyl-silanyloxy)-2-[phenoxythiocarbonyloxy-(1,3,5-trimethyl-1H-pyrazol-4-yl)-methyl]-pyrrolidine-1-carboxylic acid benzyl ester (210 mg, 58%) as an oil. LC-MS: 610.0.

To a solution of (2R,4S)-4-(tert-butyl-dimethyl-silanyloxy)-2-[phenoxythiocarbonyloxy-(1,3,5-trimethyl-1H-pyrazol-4-yl)-methyl]-pyrrolidine-1-carboxylic acid benzyl ester (210 mg, 0.34 mmol) in toluene (3 mL) is added tributyl tin hydride (198 mg, 0.68 mmol) and 2,2′-azobis(2-methylpropionitrile) (28 mg, 0.17 mmol) at room temperature. The mixture is refluxed for 12 hours and, after cooled to room temperature, the mixture is concentrated under reduced pressure and purified with a silica gel column chromatography (12-100% ethyl acetate/heptane) to give (2S,4S)-4-(tert-butyl-dimethyl-silanyloxy)-2-(1,3,5-trimethyl-1H-pyrazol-4-ylmethyl)-pyrrolidine-1-carboxylic acid benzyl ester (120 mg, 77%) as a colorless oil. LC-MS: 457.9.

### Step g: Preparation of (2S,4S)-4-hydroxy-2-(1,3,5-trimethyl-1H-pyrazol-4-ylmethyl)-pyrrolidine-1-carboxylic acid benzyl ester

To a solution of (2S,4S)-4-(tert-butyl-dimethyl-silanyloxy)-2-(1,3,5-trimethyl-1H-pyrazol-4-ylmethyl)-pyrrolidine-1-carboxylic acid benzyl ester (945 mg, 2.1 mmol) in tetrahydrofuran (10 mL) is added tetrabutylammonium fluoride (4.2 mL, 1.0 M in tetrahydrofuran) slowly. The reaction mixture is warmed to room temperature and stirred for 1 hour. A saturated sodium bicarbonate solution is and the layers separated. The aqueous phase is extracted three times with ethyl acetate (15 mL) and the organic phases are combined, washed with a saturated sodium chloride solution, dried with magnesium sulfate, filtered, and concentrated. The residue is purified with a silica gel column chromatography to give (2S,4S)-4-hydroxy-2-(1,3,5-trimethyl-1H-pyrazol-4-ylmethyl)-pyrrolidine-1-carboxylic acid benzyl ester (478 mg, 67%) as a colorless sticky oil. LC-MS: 344.1.

Following procedures analogous to those described in Example, Steps 1(c) and (f) the following compounds are prepared:

| Cpd | Name | MS |
|---|---|---|
| 31 | (E)-N-Hydroxy-3-{4-[(2S,4S)-4-hydroxy-2-(1,3,5-trimethyl-1H-pyrazol-4- | 385.2233 |
| | ylmethyl)-pyrrolidin-1-ylmethyl]-phenyl}-acrylamide | |
| 33 | (E)-3-{4-[(2S,4S)-2-(3,5-Dimethyl-1-phenyl-1H-pyrazol-4-ylmethyl)-4- | 447.2384 |
| | hydroxy-pyrrolidin-1-ylmethyl]-phenyl}-N-hydroxy-acrylamide | |
| 34 | (E)-3-(4-{(2R,4S)-2-[(3,5-Dimethyl-1-phenyl-1H-pyrazol-4-yl)-hydroxy- | 463.2351 |
| | methyl]-4-hydroxy-pyrrolidin-1-ylmethyl}-phenyl)-N-hydroxy-acrylamide | |

### Example 12

### Preparation of (E)-N-hydroxy-3-{4-[(R)-2-(1,3,5-trimethyl-1H-pyrazol-4-ylmethyl)-pyrrolidin-1-ylmethyl]-phenyl}-acrylamide (32)

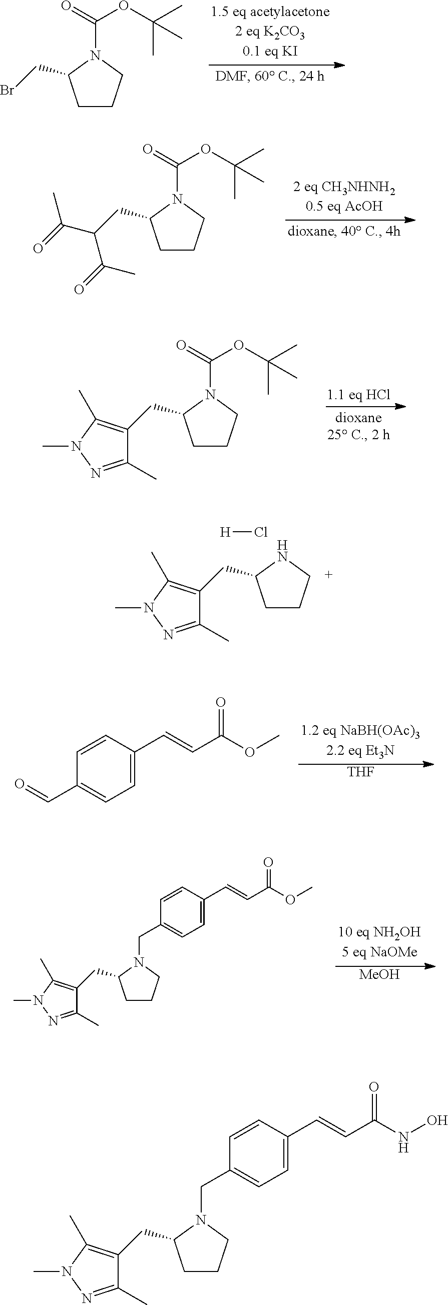

Following literature procedures (J. Med. Chem., 1992, 35:2610-2617, J. Org. Chem., 1983, 48(22):4058-4067, and Tet. Lett., 2006, 8069-8076), (R)-2-(1,3,5-trimethyl-1H-pyrazol-4-ylmethyl)-pyrrolidine-1-carboxylic acid tert-butyl ester is prepared.

To a stirred solution of (R)-2-(1,3,5-trimethyl-1H-pyrazol-4-ylmethyl)-pyrrolidine-1-carboxylic acid tert-butyl ester (0.55 g, 1.87 mmol) in dioxane (2 mL) is added 0.5 mL of 6 N hydrochloric acid in dioxane (1.1 eq., 2.06 mmol). The reaction is stirred overnight, diluted with diethyl ether, and filtered to yield 1,3,5-trimethyl-4-(R)-1-pyrrolidin-2-ylmethyl-1H-pyrazole hydrochloride (430 mg) as a white solid.

Following procedures analogous to those described in Example 1, Steps (e) and (f), (E)-N-hydroxy-3-{4-[(R)-2-(1,3,5-trimethyl-1H -pyrazol-4-ylmethyl)-pyrrolidin-1-ylmethyl]-phenyl}-acrylamide (32) is prepared. LCMS: 369.1.

### Example 13

### Preparation of (E)-3-{6-[(2S,4R)-4-fluoro-2-(1,3,5-trimethyl-1H-pyrazol-4-ylmethyl)-pyrrolidin-1-ylmethyl]-pyridin-3-yl}-N-hydroxy-acrylamide (35)

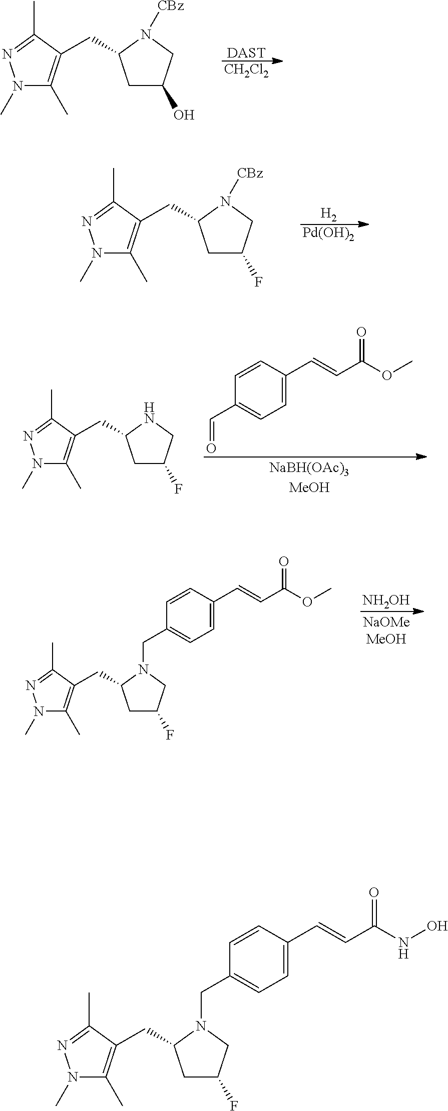

To a solution of (diethylamino)sulfur trifluoride (145 mg, 0.9 mmol) in methylene chloride (1.2 mL) at −78° C. is added a solution of (2S,4S)-4-hydroxy-2-(1,3,5-trimethyl-1H-pyrazol-4-ylmethyl)-pyrrolidine-1-carboxylic acid benzyl ester (206 mg, 0.6 mmol) in dichloromethane (0.6 mL). The reaction mixture is stirred at −78° C. for 12 hours, warmed to room temperature, and stirred for 3 hours. The reaction mixture is quenched with a saturated sodium bicarbonate solution and magnesium sulfate in small portions is added. The solution is separated and the aqueous solution is extracted with dichloromethane (15 mL). The organic phases are combined, washed with a saturated solution of sodium chloride, dried over sodium sulfate, filtered, and concentrated. The residue is purified with a silica gel column chromatography (0-15% methanol/dichloromethane) to give (2S,4R)-4-fluoro-2-(1,3,5-trimethyl-1H-pyrazol-4-ylmethyl)-pyrrolidine-1-carboxylic acid benzyl ester (147 mg, 71% yield). LC-MS: 346.4.

Following procedures analogous to those described in Example 1, Steps OHO, (E)-3-{6-[(2S,4R)-4-fluoro-2-(1,3,5-trimethyl-1H-pyrazol-4-ylmethyl)-pyrrolidin-1-ylmethyl]-pyridin-3-yl}-N-hydroxy-acrylamide (37) is prepared. FIRMS: 387.2203.

### Example 14

### Preparation of (E)-N-hydroxy-3-{4-[3-(2-methyl-1H-indol-3-yl)-pyrrolidin-1-ylmethyl]-phenyl}-acrylamide

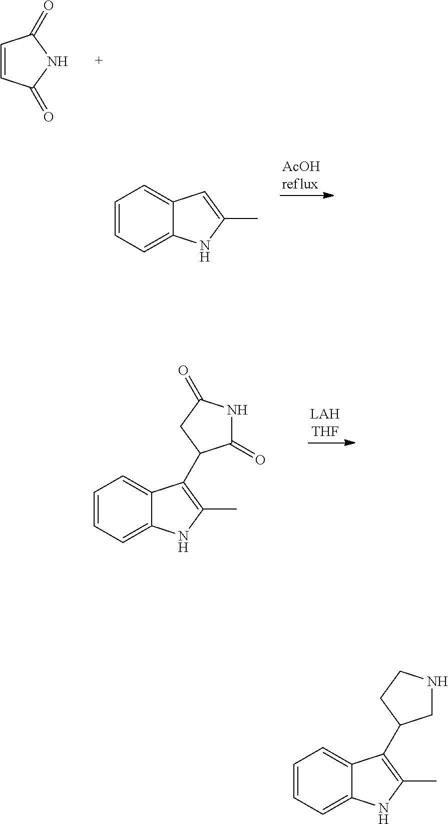

A solution of 2-methylindole (5.64 g, 56.9 mmol) and maleimide (7.78 g, 58.2 mmol) in acetic acid (50 mL) is heated to reflux under nitrogen atmosphere. The reaction mixture is concentrated in vacuo and diluted with ethyl acetate (300 mL). The organic phase is washed with water (2×100 mL), a saturated aqueous sodium bicarbonate solution (3×150 mL), dried over magnesium sulfate, filtered, and concentrated in vacuo. The residue is purified by a silica gel column chromatography to provide 3-(2-methyl-1H-indol-3-yl)-pyrrolidine-2,5-dione (3.6 g, 31% yield).

To a suspension of lithium aluminium hydride (3.48 g, 88.9 mmol) in tetrahydrofuran (25 mL, cooled with ice-bath during suspension formation) is added a solution of 3-(2-methyl-1H-indol-3-yl)-pyrrolidine-2,5-dione (1.82 g, 7.97 mmol) in tetrahydrofuran (50 mL) slowly. The reaction mixture is heated to reflux under nitrogen for 8 hours, cooled to 0° C., and treated with ethyl acetate (7 mL) and water (3.5 mL). The resulting mixture is stirred at room temperature, treated with an aqueous sodium hydroxide (6.6 mL, 1N), heated to reflux, treated with water (11 mL), stirred for 1 hour, cooled to room temperature, and filtered. The filtrate is concentrated in vacuo to give 2-methyl-3-pyrrolidin-3-yl-1H-indole, which is used for the subsequent reaction without purification.

Following procedures analogous to those described in Example 1, Steps (e) and (f), (E)-N-hydroxy-3-{4-[3-(2-methyl-1H-indol-3-yl)-pyrrolidin-1-ylmethyl]-phenyl}-acrylamide (36) is prepared. LCMS: 375.91.

(E)-N-Hydroxy-3-{4-[3-(2-methyl-1H-indol-3-yl)-pyrrolidin-1-ylmethyl]-phenyl}-acrylamide (36) is subjected to a chiral HPLC separation (Chiralpak AD-H column (5 uM, 250×4.6 mm), n-hexanes:isopropylalcohol 55:45 (volume)) to provide (E)-N-hydroxy-3-{4-[3-(2-methyl-1H-indol-3-yl)-pyrrolidin-1-ylmethyl]-phenyl}-acrylamide in enantiomerically pure forms (37 and 38).

### Example 15

### Preparation of (E)-N-hydroxy-3-{4-[(R)-2-(3-phenyl-[1,2,4]oxadiazol-5-ylmethyl)-pyrrolidin-1-ylmethyl]-phenyl}-acrylamide (39)

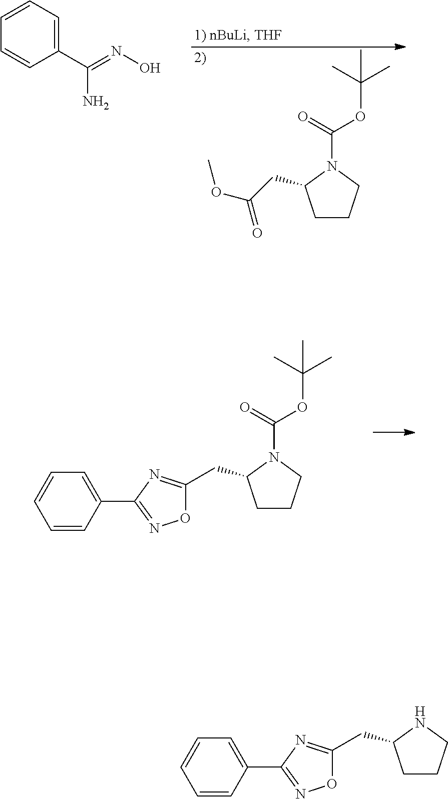 [SMILES: N/C(=N\O)c1ccccc1 ; COC(=O)C[C@H]1CCCN1C(=O)OC(C)(C)C ; CC(C)(C)OC(=O)N1CCC[C@@H]1Cc1nc(-c2ccccc2)no1 ; c1ccc(-c2noc(C[C@H]3CCCN3)n2)cc1]

To a solution of benxamidoxime (603 mg, 4.43 mmol) in tetrahydrofuran (8 mL) is added a solution of n-butyl lithium in hexanes (3.5 mL, 2.5 M, 8.8 mmol) at 0° C. and the solution is stirred for 1 h. A solution of (R)-2-methoxycarbonylmethyl-pyrrolidine-1-carboxylic acid tert-butyl ester (0.450 g, 1.85 mmol) in tetrahydrofuran (1.2 mL) is added and the resulting mixture is warmed to room temperature. The reaction mixture is treated with water (50 mL) and extracted with ethyl acetate (3×70 mL). The organic layers are combined, dried over magnesium sulfate, filtered, and concentrated in vacuo. The residue is purified by a silica gel column chromatography to give (R)-2-(3-phenyl-[1,2,4]oxadiazol-5-ylmethyl)-pyrrolidine-1-carboxylic acid tert-butyl ester (328 mg, 54% yield). IC-MS: 330.1.

A solution of (R)-2-(3-phenyl-[1,2,4]oxadiazol-5-ylmethyl)-pyrrolidine-1-carboxylic acid tert-butyl ester (328 mg, 0.995 mmol) in dioxane (3 mL) is treated with a solution of hydrochloric acid in dioxane (1.5 mL, 4 M, 6.0 mmol) and the resulting mixture is stirred at room temperature. After the reaction is deemed complete, it is concentrated and treated with diethyl ether. The solid is collected to provide 3-phenyl-5-(R)-1-pyrrolidin-2-ylmethyl-[1,2,4]oxadiazole (223 mg, 84% yield).

Following procedures analogous to those described in Example 1, Steps (e) and (f), (E)-N-hydroxy-3-{4-[(R)-2-(3-phenyl-[1,2,4]oxadiazol-5-ylmethyl)-pyrrolidin-1-ylmethyl]-phenyl}-acrylamide (39) is prepared. LC-MS 404.3 (M+1)

### Example 16

### Preparation of (E)-N-hydroxy-3-{4-[(2R,4R)-4-hydroxy-2-(4-pyridin-3-yl-[1,2,3]triazol-1-ylmethyl)-pyrrolidin-1-ylmethyl]-phenyl}-acrylamide (41)

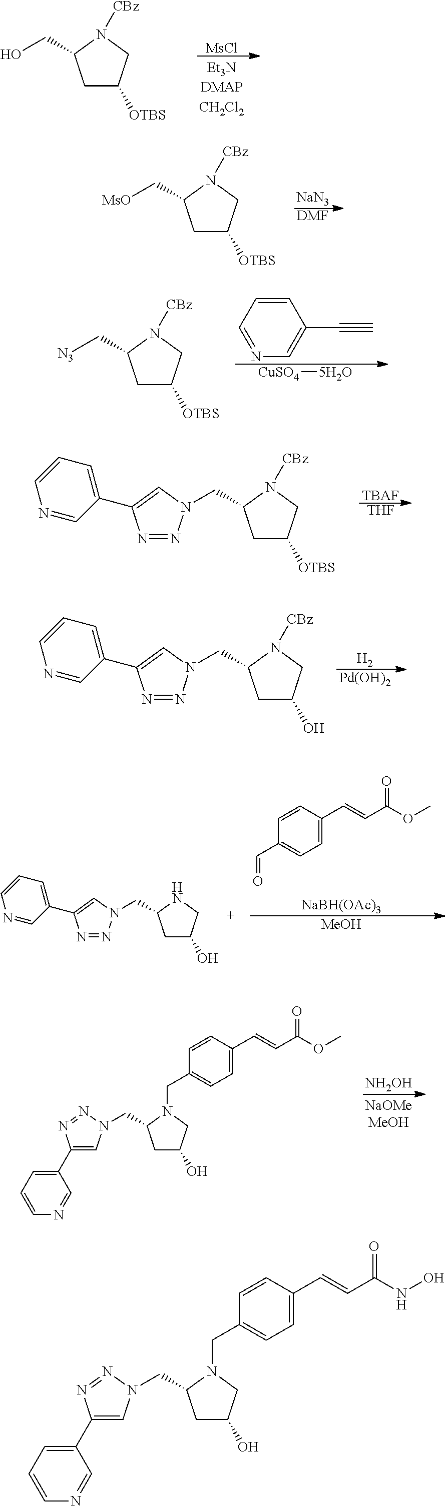

### Step a: Preparation of (2R,4R)-4-(tert-butyl-dimethyl-silanyloxy)-2-methanesulfonyloxymethyl-pyrrolidine-1-carboxylic acid benzyl ester

To a solution of (2R,4R)-4-(tert-butyl-dimethyl-silanyloxy)-2-hydroxymethyl-pyrrolidine-1-carboxylic acid benzyl ester (15 g, 41 mmol) and triethylamine (11.4 mL, 82 mmol) in dichloromethane (160 mL) is added methanesulfonyl chloride (4.7 mL, 61 mmol) at 0° C. The resulting mixture is stirred for 3 hours and poured into water and the mixture is extracted with ethyl acetate. The organic layers are combined, washed with a saturated solution of sodium chloride, dried over magnesium sulfate, filtered, and concentrated in vacuo to afford (2R,4R)-4-(tert-butyl-dimethyl-silanyloxy)-2-methanesulfonyloxymethyl-pyrrolidine-1-carboxylic acid benzyl ester in quantitative yield. The crude product is used in the subsequent step without further purification. LC-MS: 444.2.

### Step b: Preparation of (2R,4R)-2-azidomethyl-4-(tert-butyl-dimethyl-silanyloxy)-pyrrolidine-1-carboxylic acid benzyl ester

Sodium azide (1.3 g, 20 mmol) is added to a stirred solution of (2R,4R)-4-(tert-butyl-dimethyl-silanyloxy)-2-methanesulfonyloxymethyl-pyrrolidine-1-carboxylic acid benzyl ester (1.8 g, 4 mmol) in dry N,N-dimethylformamide (20 mL) at room temperature. The reaction is stirred at 90° C. for 4 hours and concentrated. The residue is partitioned between a mixture of saturated sodium bicarbonate solution (15 mL) and ethyl acetate (15 mL). The aqueous phase is extracted three times with ethyl acetate (60 mL). The organic phases are combined, washed with brine, dried over magnesium sulfate, filtered, and concentrated. The residue is purified with a silica gel column chromatography (12-100% ethyl acetate/heptane) to provide (2R,4R)-2-azidomethyl-4-(tert-butyl-dimethyl-silanyloxy)-pyrrolidine-1-carboxylic acid benzyl ester (1.35 g, 87%) as a colorless oil. LC-MS: 391.2.

### Step c: Preparation of (2R,4R)-4-(tert-butyl-dimethyl-silanyloxy)-2-(4-pyridin-3-yl-[1,2,3]triazol-1-ylmethyl)-pyrrolidine-1-carboxylic acid ethyl ester

(2R,4R)-2-Azidomethyl-4-(tert-butyl-dimethyl-silanyloxy)-pyrrolidine-1-carboxylic acid benzyl ester (675 mg, 1.7 mmol) and 3-ethynyl-pyridine (180 mg, 1.7 mmol) are suspended in a mixture of water and tert-butanol (8 mL, 1:1). Sodium ascorbate (0.17 mmol, 170 uL of freshly prepared 1 M solution in water) is added, followed by copper(II) sulfate pentahydrate (4.3 mg, 0.017 mmol, in 100 uL of water). The mixture is stirred vigorously overnight, diluted with water (50 mL), and extracted three times with ethyl acetate (90 mL). The organic phases are combined, washed with a saturated sodium chloride solution, dried over magnesium sulfate, filtered, and concentrated. The residue is purified with a silica gel column chromatography (12-100% ethyl acetate/heptanes) to provide (2R,4R)-4-(tert-butyl-dimethyl-silanyloxy)-2-(4-pyridin-3-yl-[1,2,3]triazol-1-ylmethyl)-pyrrolidine-1-carboxylic acid ethyl ester (230 mg, 23%) as a colorless oil. LC-MS: 493.5.

Following procedures analogous to those described in Examples 11, Step (g), and 1, Steps (d)-(f), (E)-N-hydroxy-3-{4-[(2R,4R)-4-hydroxy-2-(4-pyridin-3-yl-[1,2,3]triazol-1-ylmethyl)-pyrrolidin-1-ylmethyl]-phenyl}-acrylamide (43, HRMS: 421.2004) and (E)-N-hydroxy-3-{4-[(2R,4R)-4-hydroxy-2-(4-phenyl-[1,2,3]triazol-1-ylmethyl)-pyrrolidin-1-ylmethyl]phenyl}-acrylamide (42, HRMS: 420.2036) are prepared.

### Example 17

### Preparation of (E)-3-{4-[(R)-2-(4-benzyl-[1,2,3]triazol-1-ylmethyl)-pyrrolidin-1-ylmethyl]-phenyl}-N-hydroxy-acrylamide (43)

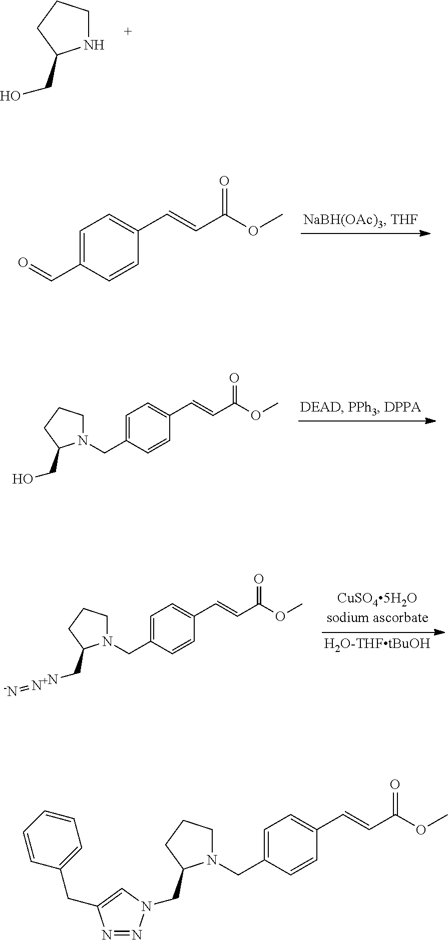

A mixture of (R)-1-pyrrolidin-2-yl-methanol (5 g, 49.4 mmol) and (E)-3-(4-formyl-phenyl)-acrylic acid methyl ester (9108 g, 48.46 mmol) in tetrahydrofuran (250 mL) is treated with sodium triactoxyborohydride (16.69 g, 79.09 mmol) and the resulting mixture is stirred at room temperature overnight. A saturated solution of ammonium chloride is added and the resulting mixture is extracted with ethyl acetate. The organic layers are combined, washed with water, dried over sodium sulfate, filtered and concentrated. The residue is purified by a silica gel column chromatography to give (E)-3-[4-((R)-2-hydroxymethyl-pyrrolidin-1-ylmethyl)-phenyl]-acrylic acid methyl ester (8.77 g, 65% yield).

To a solution of triphenylphosphine (16.1 g, 60.8 mmol) in tetrahydrofuran (50 mL) is added diethyl azodicarboxylate (11.22 g, 64.4 mmol) at 0° C. and the resulting solution is stirred at room temperature for 15 minutes. A solution of (E)-3-[4-((R)-2-hydroxymethyl-pyrrolidin-1-ylmethyl)-phenyl]-acrylic acid methyl ester (4.625 g, 16.8 mmol) in tetrahydrofuran (50 mL) is added and the resulting mixture is stirred for 20 minutes. Diphenylphosphoryl azide (14.4 mL, 64.6 mmol) is added and the resulting solution is stirred for 23 hours, treated with water (1.5 mL), and concentrated in vacuo. The residue is purified with a silica gel column chromatography to provide (E)-3-[4-((R)-2-azidomethyl-pyrrolidin-1-ylmethyl)-phenyl]-acrylic acid methyl ester (1.26 g, 25% yield).

To a solution of (E)-3-[4-((R)-2-azidomethyl-pyrrolidin-1-ylmethyl)-phenyl]-acrylic acid methyl ester (110 mg, 0.366 mmol) and prop-2-ynyl-benzene (42.5 mg, 0.366 mmol) in water-tetrahydrofuran-t-BuOH (v:v:v=1:1:1, 3 mL) are added sodium ascorbate (1.5 mL, 1 M solution in water) and copper (II) sulfate pentahydrate. The resulting mixture is stirred for 8 hours and treated with a polymer-bound copper scavenger (˜15 mg) overnight. The mixture is filtered, and the filtrate is concentrated and purified with a silica gel column chromatography to give (E)-3-{4-[(R)-2-(4-benzyl-[1,2,3]triazol-1-ylmethyl)-pyrrolidin-1-ylmethyl]-phenyl}-N-hydroxy-acrylamide (48 mg, 31.5% yield).

Following procedures analogous to those described in Examples 17 and 1, Step (f), the following compounds are prepared:

| Cpd | Name | MS |
|---|---|---|
| 42 | (E)-3-{4-[(R)-2-(4-Cyclohexylmethyl-[1,2,3]triazol-1-ylmethyl)-pyrrolidin- | 424.5 |
| | 1-ylmethyl]-phenyl}-N-hydroxy-acrylamide | |
| 43 | (E)-3-{4-[(R)-2-(4-Benzyl-[1,2,3]triazol-1-ylmethyl)-pyrrolidin-1- | 417.2252 |
| | ylmethyl]-phenyl}-N-hydroxy-acrylamide | |
| 44 | (E)-N-Hydroxy-3-(4-{(R)-2-[4-(1-hydroxy-1-methyl-ethyl)-[1,2,3]triazol-1- | 386.5 |
| | ylmethyl]-pyrrolidin-1-ylmethyl}-phenyl)-acrylamide | |
| 45 | (E)-N-Hydroxy-3-(4-{(R)-2-[4-(4-hydroxy-tetrahydro-pyran-4-yl)- | 428.5 |
| | [1,2,3]triazol-1-ylmethyl]-pyrrolidin-1-ylmethyl}-phenyl)-acrylamide | |
| 46 | (E)-N-Hydroxy-3-{4-[(R)-2-(4-hydroxymethyl-[1,2,3]triazol-1-ylmethyl)- | 358.4 |
| | pyrrolidin-1-ylmethyl]-phenyl}-acrylamide | |

### Example 18

### Preparation of (E)-N-hydroxy-3-[4-((R)-2-pyrazol-1-ylmethyl-pyrrolidin-1-ylmethyl)-phenyl]-acrylamide (49)

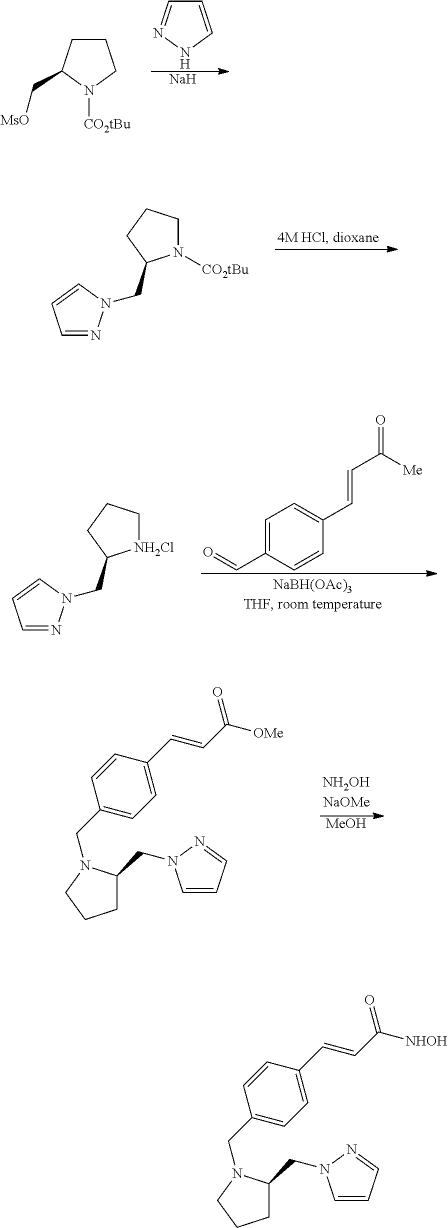

To a stirred suspension of sodium hydride (107 mg, 2.69 mmol, 1.5 eq.) in 3.5 mL of N,N-dimethylformamide is added pyrazole (182 mg, 2.69 mmol, 1.5 equivalents). (R)-2-Methanesulfonyloxymethyl-pyrrolidine-1-carboxylic acid tert-butyl ester (500 mg, 1.79 mmol, 1.0 equivalent) in dimethylformamide (2.5 mL) is added. The solution is heated at 70° C. for 3 hours. After cooling to room temperature, water is added and the reaction mixture is extracted with ethyl acetate. The organic layers are combined, washed with brine, dried with sodium sulfate, filtered, and concentrated under reduced pressure. The residue is purified by a silica gel column chromatography (0-100% ethyl acetate/heptanes gradient) to provide (R)-2-pyrazol-1-ylmethyl-pyrrolidine-1-carboxylic acid tert-butyl ester (340 mg, 75% yield) as a clear oil.

To a solution of (R)-2-pyrazol-1-ylmethyl-pyrrolidine-1-carboxylic acid tert-butyl ester (340 mg) in diethyl ether is added hydrochloric acid (2 mL, 2 M) in diethyl ether. The solution is stirred for 2 hours and the precipitate is collected by filtration and washed with diethyl ether to provide 1-(R)-1-pyrrolidin-2-ylmethyl-1H-pyrazole hydrogen chloride salt (240 mg, 94% yield) as a white solid.

Following procedures analogous to those in Examples 18 and 1, Steps (e) and (0, the following compounds are prepared:

| Cpd | Name | MS |
|---|---|---|
| 47 | (E)-N-Hydroxy-3-[4-((R)-2-indazol-1-ylmethyl-pyrrolidin-1-ylmethyl)- | 377.1980 |
| | phenyl]-acrylamide | |
| 48 | (E)-N-Hydroxy-3-[4-((R)-2-indazol-2-ylmethyl-pyrrolidin-1-ylmethyl)- | 377.1980 |
| | phenyl]-acrylamide | |
| 49 | (E)-N-Hydroxy-3-[4-((R)-2-pyrazol-1-ylmethyl-pyrrolidin-1-ylmethyl)- | 327.1819 |
| | phenyl]-acrylamide | |
| 50 | (E)-3-{4-[(R)-2-(3,5-Dimethyl-pyrazol-1-ylmethyl)-pyrrolidin-1-ylmethyl]- | 355.2131 |
| | phenyl}-N-hydroxy-acrylamide | |
| 51 | (E)-3-{4-[(R)-2-(3,5-Bis-trifluoromethyl-pyrazol-1-ylmethyl)-pyrrolidin-1- | 463.1568 |
| | ylmethyl]-phenyl}-N-hydroxy-acrylamide | |
| 52 | (E)-3-{4-[(2R,4R)-2-(3,5-Bis-trifluoromethyl-pyrazol-1-ylmethyl)-4-hydroxy- | 479.1530 |
| | pyrrolidin-1-ylmethyl]-phenyl}-N-hydroxy-acrylamide | |
| 53 | (E)-3-{4-[(2R,4R)-2-(3,5-Dimethyl-pyrazol-1-ylmethyl)-4-hydroxy- | 371.4 |
| | pyrrolidin-1-ylmethyl]-phenyl}-N-hydroxy-acrylamide | |

### Example 19

### HDAC Inhibition Assay

The baculovirus donor vector pFB-GSTX3 is used to generate a recombinant baculovirus that expresses the HDAC polypeptide. Transfer vectors containing the HDAC coding region are transfected into the DH10Bac cell line (GIBCO) and plated on selective agar plates. Colonies without insertion of the fusion sequence into the viral genome (carried by the bacteria) are blue. Single, white colonies are picked and viral DNAs (bacmid) are isolated from the bacteria by standard plasmid purification procedures. Sf9 cells or Sf21 (American Type Culture Collection) cells are then transfected in 25 cm³ flasks with the viral DNA using Cellfectin reagent.

### Determination of Small Scale Protein Expression in Sf9 Cells

Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (at an approximately MOI of 5). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

### Purification of GST-Tagged Proteins

The centrifuged cell lysate is loaded onto a 2 mL glutathione-sepharose column (Pharmacia) and is washed thrice with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

### Enzyme Activity Measurement

HDAC assays with purified GST-HDAC protein are carried out in a final volume of 30 μL containing 15 ng of GST-HDAC protein, 20 mM tris-HCl, pH 7.5, 1 mM MnCl₂, 10 mM MgCl₂, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors. The assay is carried out in 96-well plates at ambient temperature for 15 minutes under conditions described below and terminated by the addition of 20 μL of 125 mM EDTA. Subsequently, 40 μL of the reaction mixture are transferred onto EMMOBILON-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, a vacuum is connected and each well-rinsed with 200 μL 0.5% H₃PO₄. Membranes are removed and washed four times on a shaker with 1.0% H₃ PO₄, once with ethanol. Membranes are counted after drying at ambient temperature, mounting in Packard TopCount 96-well frame, and addition of 10 μL/well of MICROSCINT™ (Packard). IC50 values are calculated by linear regression analysis of the percentage inhibition of each compound in duplicate, at 4 concentrations (usually 0.01, 0.1, 1 and 10 μM).

### IC₅₀ Calculations

Input: 3×4 μL stopped assay on IMMOBILON membrane, not washed
Background (3 wells): assay with H₂O instead of enzyme
Positive control (4 wells): 3% DMSO instead of compound
Bath control (1 well): no reaction mix

IC50 values are calculated by logarithmic regression analysis of the percentage inhibition of each compound at 4 concentrations (usually 3- or 10-fold dilution series starting at 10 μM). In each experiment, the actual inhibition by reference compound is used for normalization of IC₅₀ values to the basis of an average value of the reference inhibitor:

Normalized IC50=measured IC50 average ref. IC50/measured ref. IC50

Example: Reference inhibitor in experiment 0.4 μM, average 0.3 μM,

Test compound in experiment 1.0 μM, normalization: 0.3/0.4=0.75 μM

For example, known HDAC inhibitors or a synthetic derivative thereof may be used as reference compounds.

Using this protocol, the compounds of the present teachings are found to show IC50 values for HDAC inhibition in the range from about 0.0004 μM to about 100 μM, or about 0.0004 μM to about 50 μM, including, for example, the range from about 0.0004 μM to about 2 μM or less.

Table 2 provides assay results of exemplified compounds.

TABLE 2

| | HDAC-1 | HCT116 |
|---|---|---|
| Cpd No. | IC₅₀ (nM) | IC₅₀ (nM) |
| 1 | 10 | 100 |
| 2 | 0.65 | 3 |
| 3 | 2 | 1.4 |
| 4 | 0.65 | 0.4 |
| 5 | 8 | |
| 6 | 5 | |
| 7 | | |
| 8 | 10 | 83 |
| 9 | 1.3 | 3 |
| 10 | 0.7 | 0.6 |
| 11 | 56 | 560 |
| 12 | 38 | 520 |
| 13 | | |
| 14 | 1.3 | 2.4 |
| 15 | 1 | 3 |
| 16 | 380 | |
| 17 | 210 | 950 |
| 18 | 350 | 7,000 |
| 19 | 36 | 100 |
| 20 | 0.6 | 1 |
| 21 | 0.7 | 1 |
| 22 | 0.45 | 4 |
| 23 | 1.7 | 8 |
| 24 | 1.2 | 0.5 |
| 25 | 1.2 | 0.4 |
| 26 | 1.7 | |
| 27 | | |
| 28 | 1.7 | |
| 29 | | |
| 30 | | |
| 31 | 2 | 16 |
| 32 | | |
| 33 | 0.7 | |
| 34 | 27 | |
| 35 | | |
| 36 | | |
| 37 | | |
| 38 | | |
| 39 | 20 | |
| 40 | | |
| 41 | 170 | |
| 42 | | |
| 43 | | |
| 44 | | |
| 45 | | |
| 46 | | |
| 47 | 1.8 | 1 |
| 48 | 7 | 9 |
| 49 | 15 | 26 |
| 50 | 3 | 4 |
| 51 | 39 | 31 |
| 52 | 37 | 52 |
| 53 | | |

As those skilled in the art will appreciate, numerous changes and modifications can be made to the above-described embodiments of the present teachings without departing from the spirit of the present teachings. It is intended that all such variations fall within the scope of the present teachings.

1. A compound of Formula I: 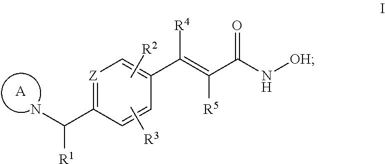
or a pharmaceutically acceptable salt, hydrate, or ester thereof,
wherein:
ring A, including the nitrogen atom (N), is a 5 membered cycloheteroalkyl group optionally substituted with 1-4 —Y—R⁶ groups;
Y, at each occurrence, is a) a divalent C₁₋₁₀ alkyl group, b) a divalent C₂₋₁₀ alkenyl group, c) a divalent C₂₋₁₀ alkynyl group, or d) a covalent bond, wherein each of a)-c) optionally is substituted with 1-4 R⁹;
Z is a) CH or b) N;
R¹ is a) H, b) a C₁₋₁₀ alkyl group, c) a C₂₋₁₀ alkenyl group, d) a C₂₋₁₀ alkynyl group,
e) a C₃₋₁₄ cycloalkyl group, or f) a 3-14 membered cycloheteroalkyl group, wherein each of b)-f) optionally is substituted with 1-4 -L-R⁹ groups;
R², R³, R⁴, and R⁵ independently are a) H or b) halogen;
R⁶, at each occurrence, is a) H, b) halogen, c) —OR⁷, d) —NR⁷R⁸, e) a C₁₋₁₀ alkyl group,
f) a C₂₋₁₀ alkenyl group, g) a C₂₋₁₀ alkynyl group, h) a C₃₋₁₄ cycloalkyl group, i) a C₆₋₁₄ aryl group, j) a 3-14 membered cycloheteroalkyl group, or k) a 5-14 membered heteroaryl group, wherein each of e)-k) optionally is substituted with 1-4 -L-R⁹ groups, or
two —Y—R⁶ groups, taken together with the atom to which each —Y—R⁶ group is attached and any intervening ring atoms, form a) a C₃₋₁₄ cycloalkyl group or b) a 3-14 membered cycloheteroalkyl group, wherein each of a)-b) optionally is substituted with 1-4 R⁹ groups;
R⁷ and R⁸, at each occurrence, independently are a) H, b) —C(O)R¹¹, c) —S(O)ₘ—R¹¹,
d) a C₁₋₁₀ alkyl group, e) a C₂₋₁₀ alkenyl group, f) a C₂₋₁₀ alkynyl group, g) a C₃₋₁₄ cycloalkyl group, h) a C₆₋₁₄ aryl group, i) a 3-14 membered cycloheteroalkyl group, or
j) a 5-14 membered heteroaryl group, wherein each of d)-j) optionally is substituted with 1-4 -L-R⁹ groups;
R⁹, at each occurrence, is a) halogen, b) —CN, c) —NO₂, d) oxo, e) ═N-L-R¹⁰,
f) —O-L-R¹⁰, g) —NR¹⁶-L-R¹⁰, h) a C₁₋₁₀ alkyl group, i) a C₁₋₁₀ haloalkyl group,
j) a C₂₋₁₀ alkenyl group, k) a C₂₋₁₀ alkynyl group, 1) a C₃₋₁₄ cycloalkyl group, m) a C₆₋₁₄ aryl group, n) a 3-14 membered cycloheteroalkyl group, or o) a 5-14 membered heteroaryl group, wherein each of h)-o) optionally is substituted with 1-4 -L-R¹³ groups;
R¹⁰, at each occurrence, is a) H, b) —OR¹¹, c) —NR¹¹R¹², d) —C(O)R¹¹, e) —S(O)ₘ—R¹¹,
f) a C₁₋₁₀ alkyl group, g) a C₂₋₁₀ alkenyl group, h) a C₂₋₁₀ alkynyl group, i) a C₃₋₁₄ cycloalkyl group, j) a C₆₋₁₄ aryl group, k) a 3-14 membered cycloheteroalkyl group, or
l) a 5-14 membered heteroaryl group, wherein each of f)-l) optionally is substituted with 1-4 -L-R¹³ groups;
R¹¹ and R¹², at each occurrence, independently are a) H, b) a C₁₋₁₀ alkyl group, c) a C₂₋₁₀ alkenyl group, d) a C₂₋₁₀ alkynyl group, e) a C₃₋₁₄ cycloalkyl group, f) a C₆₋₁₄ aryl group, g) a 3-14 membered cycloheteroalkyl group, or h) a 5-14 membered heteroaryl group, wherein each of b)-h) optionally is substituted with 1-4 -L-R¹³ groups;
R¹³, at each occurrence, is a) halogen, b) —CN, c) —NO₂, d) oxo, e) —OH, f) —NH₂,
g) —NH(C₁₋₁₀ alkyl), h) —N(C₁₋₁₀ alkyl)₂, i) —CHO, j) —C(O)—C₁₋₁₀ alkyl, k) —C(O)OH,
l) —C(O)—O(C₁₋₁₀ alkyl), m) —C(O)SH, n) —C(O)—SC₁₋₁₀ alkyl, o) —C(O)NH₂, p) —C(O)NH(C₁₋₁₀ alkyl), q) —C(O)N(C₁₋₁₀ alkyl)₂, r) —C(S)H, s) —C(S)—C₁₋₁₀ alkyl,
t) —C(S)NH₂, u) —C(S)NH(C₁₋₁₀ alkyl), v) —C(S)N(C₁₋₁₀ alkyl)₂, w) —C(NH)H, x) —C(NH)(C₁₋₁₀ alkyl), y) —C(NH)NH₂, z) —C(NH)NH(C₁₋₁₀ alkyl),
aa) —C(NH)N(C₁₋₁₀ alkyl)₂, ab) —C(NC₁₋₁₀ alkyl)H, ac) —C(NC₁₋₁₀ alkyl)-C₁₋₁₀ alkyl,
ad) —C(NC₁₋₁₀ alkyl)NH(C₁₋₁₀ alkyl), ae) —C(NC₁₋₁₀ alkyl)N(C₁₋₁₀ alkyl)₂, af) —S(O)ₘ—H, ag) —S(O)ₘ—C₁₋₁₀ alkyl, ah) —S(O)₂OH, ai) —S(O)ₘ—OC₁₋₁₀ alkyl, aj) —S(O)ₘ—NH₂,
ak) —S(O)ₘNH(C₁₋₁₀ alkyl), al) —S(O)—N(C₁₋₁₀ alkyl)₂, am) —Si(C₁₋₁₀ alkyl)₃, an) a C₁₋₁₀ alkyl group, ao) a C₂₋₁₀ alkenyl group, ap) a C₂₋₁₀ alkynyl group, aq) a C₁₋₁₀ alkoxy group, ar) a C₁₋₁₀ haloalkyl group, as) a C₃₋₁₄ cycloalkyl group,
at) a C₆₋₁₄ aryl group, au) a 3-14 membered cycloheteroalkyl group, or av) a 5-14 membered heteroaryl group;
L, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, e) a divalent C₁₋₁₀ alkoxy group, or f) a covalent bond; and
m, at each occurrence, is 0, 1, or 2.

2. The compound of claim 1, or a pharmaceutically acceptable salt, hydrate, or ester thereof, wherein two —Y—R⁶ groups, taken together with the atom to which each —Y—R⁶ group is attached and any intervening ring atoms, form a C₃₋₁₄ cycloalkyl group optionally substituted with 1-4 R⁹ groups wherein R⁹ is as defined in claim 1.

3. The compound of claim 2, or a pharmaceutically acceptable salt, hydrate, or ester thereof, wherein the C₃₋₁₄ cycloalkyl group, taken together with ring A, is an octahydrocyclopenta[b]pyrrolyl group.

4. The compound of claim 1, or a pharmaceutically acceptable salt, hydrate, or ester thereof, the compound having Formula II: 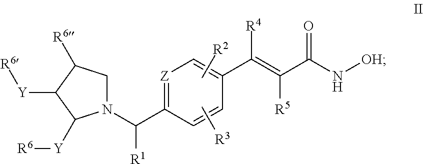
wherein:
R^{6′} and R^{6″} independently are a) H, b) halogen, c) —OR′, d) —NR⁷R⁸, e) a C₁₋₁₀ alkyl group, f) a C₂₋₁₀ alkenyl group, g) a C₂₋₁₀ alkynyl group, h) a C₃₋₁₄ cycloalkyl group,
i) a C₆₋₁₄ aryl group, j) a 3-14 membered cycloheteroalkyl group, or k) a 5-14 membered heteroaryl group, wherein each of e)-k) optionally is substituted with 1-4 -L-R⁹ groups; and
R¹, R², R³, R⁴, R⁵, R⁶, R⁷, R⁸, R⁹, L, Y, and Z are as defined in claim 1.

5. The compound of claim 1, or a pharmaceutically acceptable salt, hydrate, or ester thereof, wherein Y, at each occurrence, is a covalent bond or a divalent C₁₋₃ alkyl group optionally substituted with 1-4 R⁹ groups and R⁹ is as defined in claim 1.

6. The compound of claim 1, or a pharmaceutically acceptable salt, hydrate, or ester thereof, wherein Y, at each occurrence, is selected from —CH₂—, —CH(OH)—, or —C(O)—.

7. The compound of claim 4, or a pharmaceutically acceptable salt, hydrate, or ester thereof, wherein R⁶ and R^{6′} independently are selected from H, a C₁₋₁₀ alkyl group,
a C₃₋₁₄ cycloalkyl group, a C₆₋₁₄ aryl group, a 3-14 membered cycloheteroalkyl group, and a 5-14 membered heteroaryl group, wherein each of the C₁₋₁₀ alkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 -L-R⁹ groups, wherein L and R⁹ are as defined in claim 4.

8. The compound of claim 4, or a pharmaceutically acceptable salt, hydrate, or ester thereof, wherein R⁶ is a propyl group.

9. The compound of claim 4, or a pharmaceutically acceptable salt, hydrate, or ester thereof, wherein R⁶ and R^{6′} independently are selected from a C₆₋₁₄ aryl group, a 3-14 membered cycloheteroalkyl group, and a 5-14 heteroaryl group, each of which optionally is substituted with 1-4 -L-R⁹ groups, wherein L and R⁹ are as defined in claim 4.

10. The compound of claim 4, or a pharmaceutically acceptable salt, hydrate, or ester thereof, wherein R⁶ and R^{6′} independently are selected from a phenyl group, a pyrrolidinyl group, an indolinyl group, a pyrrolyl group, a pyrazolyl group, a triazolyl group, an oxadiazolyl group, a pyridyl group, an indolyl group, and an indazolyl group, each of which optionally is substituted with 1-4 -L-R⁹ groups, wherein L and R⁹ are as defined in claim 4.

11. The compound of claim 10, or a pharmaceutically acceptable salt, hydrate, or ester thereof, wherein R⁹ is selected from —OH, —O(C₁₋₁₀ alkyl), a Cl_{—}10 alkyl group,
a C₁₋₁₀ haloalkyl group, a C₃₋₁₄ cycloalkyl group, a C₆₋₁₄ aryl group, and a 5-14 membered heteroaryl group, wherein each of the C₁₋₁₀ alkyl groups, the C₁₋₁₀ haloalkyl group,
the C₃₋₁₄ cycloalkyl group, the C₆₋₁₄ aryl group, and the 5-14 membered heteroaryl group optionally is substituted with 1-3 R¹³ groups, wherein R¹³ is as defined in claim 1.

12. The compound of claim 4, or a pharmaceutically acceptable salt, hydrate, or ester thereof, wherein R⁶″ is selected from H, halogen, —OR⁷, and —NR⁷R⁸, wherein R⁷ and R⁸ are as defined in claim 4.

13. The compound of claim 4, or a pharmaceutically acceptable salt, hydrate, or ester thereof, wherein R^{6″} is selected from H, F, —OH, —O(C₁₋₆ alkyl), and —NH₂.

14. The compound of claim 1, or a pharmaceutically acceptable salt, hydrate, or ester thereof, the compound having Formula IIa or Formula IIb: 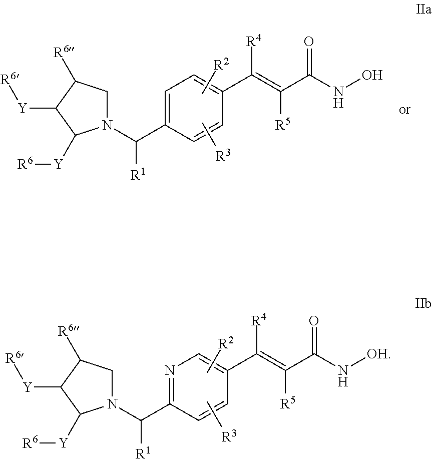
wherein R¹, R², R³, R⁴, R⁵, R⁶, R^{6′}, R^{6″}, and Y are as defined in claim 4.

15. The compound of claim 1, or a pharmaceutically acceptable salt, hydrate, or ester thereof, wherein R⁴ and R⁵ independently are selected from H, F, Cl, and Br.

16. The compound of claim 1, or a pharmaceutically acceptable salt, hydrate, or ester thereof, wherein R⁴ is H and R⁵ is selected from H and F.

17. The compound of claim 1, or a pharmaceutically acceptable salt, hydrate, or ester thereof, wherein R² and R³ independently are selected from H, F, Cl, and Br.

18. The compound of claim 1, or a pharmaceutically acceptable salt, hydrate, or ester thereof, wherein R¹ is H or a C₁₋₁₀ alkyl group optionally substituted with 1-4 R⁹ groups, wherein R⁹ is as defined in claim 1.

19. The compound of claim 1, or a pharmaceutically acceptable salt, hydrate, or ester thereof, wherein R¹ is H or a methyl group.

20. The compound of claim 1, or a pharmaceutically acceptable salt, hydrate, or ester thereof, wherein the compound is in the form of an enantiomer or a diastereomer.

21. A compound, or a pharmaceutically acceptable salt, hydrate, or ester thereof, the compound selected from:
(E)-N-hydroxy-3-{4-[(S)-2-(1H-indol-3-ylmethyl)-pyrrolidin-1-ylmethyl]-phenyl}-acrylamide,
(E)-N-hydroxy-3-{4-[(R)-2-(1H-indol-3-ylmethyl)-pyrrolidin-1-ylmethyl]-phenyl}-acrylamide,
(E)-N-hydroxy-3-{4-[(2R,3 aR,6aR)-2-(2-methyl-1H-indol-3-ylmethyl)-hexahydro-cyclopenta[b]pyrrol-1-ylmethyl]-phenyl}-acrylamide,
(E)-N-hydroxy-3-{4-[(R)-2-(2-methyl-1H-indol-3-ylmethyl)-pyrrolidin-1-ylmethyl]-phenyl}-acrylamide,
(E)-N-hydroxy-3-[4-(2-isobutyl-pyrrolidin-1-ylmethyl)-phenyl]-acrylamide,
(E)-N-hydroxy-3-[4-(2-pyridin-3-ylmethyl-pyrrolidin-1-ylmethyl)-phenyl]-acrylamide,
(E)-3-[4-(2-benzyl-pyrrolidin-1-ylmethyl)-phenyl]-N-hydroxy-acrylamide,
(E)-3-{3-fluoro-4-[(S)-2-(1H-indol-3-ylmethyl)-pyrrolidin-1-ylmethyl]-phenyl}-N-hydroxy-acrylamide,
(E)-3-{3-fluoro-4-[(R)-2-(1H-indol-3-ylmethyl)-pyrrolidin-1-ylmethyl]-phenyl}-N-hydroxy-acrylamide,
(E)-3-{3-fluoro-4-[(R)-2-(2-methyl-1H-indol-3-ylmethyl)-pyrrolidin-1-ylmethyl]-phenyl}-N-hydroxy-acrylamide,
(E)-N-hydroxy-3-{4-[(S)-2-(1H-indole-3-carbonyl)-pyrrolidin-1-ylmethyl]-phenyl}-acrylamide,
(E)-N-hydroxy-3-{4-[(R)-2-(1H-indole-3-carbonyl)-pyrrolidin-1-ylmethyl]-phenyl}-acrylamide,
(Z)-2-fluoro-N-hydroxy-3-{4-[(R)-2-(1H-indol-3-ylmethyl)-pyrrolidin-1-ylmethyl]-phenyl}-acrylamide,
(E)-N-hydroxy-3-(4-{(S)-1-[(R)-2-(2-methyl-1H-indol-3-ylmethyl)-pyrrolidin-1-yl]-ethyl}-phenyl)-acrylamide,
(E)-N-hydroxy-3-(4-{(R)-1-[(R)-2-(2-methyl-1H-indol-3-ylmethyl)-pyrrolidin-1-yl]-ethyl}-phenyl)-acrylamide,
(E)-N-hydroxy-3-{4-[1-((S)-2-pyrrolidin-1-ylmethyl-pyrrolidin-1-yl)-ethyl]-phenyl}-acrylamide,
(E)-3-{4-[(R)-2-(2,3-dihydro-indole-1-carbonyl)-pyrrolidin-1-ylmethyl]-phenyl}-N-hydroxy-acrylamide,
(E)-3-{4-[(S)-2-(2,3-dihydro-indole-1-carbonyl)-pyrrolidin-1-ylmethyl]-phenyl}-N-hydroxy-acrylamide,
(E)-3-{4-[(S)-2-(2,3-dihydro-indol-1-ylmethyl)-pyrrolidin-1-ylmethyl]-phenyl}-N-hydroxy-acrylamide,
(E)-N-hydroxy-3-{4-[(2S,4R)-4-hydroxy-2-(2-methyl-1H-indol-3-ylmethyl)-pyrrolidin-1-ylmethyl]-phenyl}-acrylamide,
(E)-N-hydroxy-3-{4-[(2S,4 S)-4-hydroxy-2-(2-methyl-1H-indol-3-ylmethyl)-pyrrolidin-1-ylmethyl]-phenyl}-acrylamide,
(E)-N-hydroxy-3-{4-[(2S,4S)-4-hydroxy-2-(1H-indol-3-ylmethyl)-pyrrolidin-1-ylmethyl]-phenyl}-acrylamide,
(E)-N-hydroxy-3-{4-[(2S,4R)-4-hydroxy-2-(1H-indol-3-ylmethyl)-pyrrolidin-1-ylmethyl]-phenyl}-acrylamide,
(E)-N-hydroxy-3-{4-[(2S,4R)-4-methoxy-2-(2-methyl-1H-indol-3-ylmethyl)-pyrrolidin-1-ylmethyl]-phenyl}-acrylamide,
(E)-N-hydroxy-3-{4-[(2S,4 S)-4-methoxy-2-(2-methyl-1H-indol-3-ylmethyl)-pyrrolidin-1-ylmethyl]-phenyl}-acrylamide,
(E)-N-hydroxy-3-{6-[(R)-2-(2-methyl-1H-indol-3-ylmethyl)-pyrrolidin-1-ylmethyl]-pyridin-3-yl}-acrylamide,
(E)-N-hydroxy-3-{6-[(2S,4R)-4-hydroxy-2-(2-methyl-1H-indol-3-ylmethyl)-pyrrolidin-1-ylmethyl]-pyridin-3-yl}-acrylamide,
(E)-N-hydroxy-3-{6-[(2S,4 S)-4-hydroxy-2-(2-methyl-1H-indol-3-ylmethyl)-pyrrolidin-1-ylmethyl]-pyridin-3-yl}-acrylamide,
(E)-3-{6-[(2S,4S)-4-amino-2-(2-methyl-1H-indol-3-ylmethyl)-pyrrolidin-1-ylmethyl]-pyridin-3-yl}-N-hydroxy-acrylamide,
(E)-3-{6-[(S)-4-fluoro-2-(2-methyl-1H-indol-3-ylmethyl)-pyrrolidin-1-ylmethyl]-pyridin-3-yl}-N-hydroxy-acrylamide,
(E)-N-hydroxy-3-{4-[(2S,4S)-4-hydroxy-2-(1,3,5-trimethyl-1H-pyrazol-4-ylmethyl)-pyrrolidin-1-ylmethyl]-phenyl}-acrylamide,
(E)-N-hydroxy-3-{4-[(R)-2-(1,3,5-trimethyl-1H-pyrazol-4-ylmethyl)-pyrrolidin-1-ylmethyl]-phenyl}-acrylamide,
(E)-3-{4-[(2S,4 S)-2-(3,5-dimethyl-1-phenyl-1H-pyrazol-4-ylmethyl)-4-hydroxy-pyrrolidin-1-ylmethyl]-phenyl}-N-hydroxy-acrylamide,
(E)-3-(4-{(2R,4 S)-2-[(3,5-dimethyl-1-phenyl-1H-pyrazol-4-yl)-hydroxy-methyl]-4-hydroxy-pyrrolidin-1-ylmethyl}-phenyl)-N-hydroxy-acrylamide,
(E)-3-{6-[(2S,4R)-4-fluoro-2-(1,3,5-trimethyl-1H-pyrazol-4-ylmethyl)-pyrrolidin-1-ylmethyl]-pyridin-3-yl}-N-hydroxy-acrylamide,
racemic (E)-N-hydroxy-3-{4-[3-(2-methyl-1H-indol-3-yl)-pyrrolidin-1-ylmethyl]-phenyl}-acrylamide,
(+)-(E)-N-hydroxy-3-{4-[3-(2-methyl-1H-indol-3-yl)-pyrrolidin-1-ylmethyl]-phenyl}-acrylamide,
(−)-(E)-N-hydroxy-3-{4-[3-(2-methyl-1H-indol-3-yl)-pyrrolidin-1-ylmethyl]-phenyl}-acrylamide,
(E)-N-hydroxy-3-{4-[(R)-2-(3-phenyl-[1,2,4]oxadiazol-5-ylmethyl)-pyrrolidin-1-ylmethyl]-phenyl}-acrylamide,
(E)-N-hydroxy-3-{4-[(2R,4R)-4-hydroxy-2-(4-phenyl-[1,2,3]triazol-1-ylmethyl)-pyrrolidin-1-ylmethyl]-phenyl}-acrylamide,
(E)-N-hydroxy-3-{4-[(2R,4R)-4-hydroxy-2-(4-pyridin-3-yl-[1,2,3]triazol-1-ylmethyl)-pyrrolidin-1-ylmethyl]-phenyl}-acrylamide,
(E)-3-{4-[(R)-2-(4-cyclohexylmethyl-[1,2,3]triazol-1-ylmethyl)-pyrrolidin-1-ylmethyl]-phenyl}-N-hydroxy-acrylamide,
(E)-3-{4-[(R)-2-(4-benzyl-[1,2,3]triazol-1-ylmethyl)-pyrrolidin-1-ylmethyl]-phenyl}-N-hydroxy-acrylamide,
(E)-N-hydroxy-3-(4-{(R)-2-[4-(1-hydroxy-1-methyl-ethyl)-[1,2,3]triazol-1-ylmethyl]-pyrrolidin-1-ylmethyl}-phenyl)-acrylamide,
(E)-N-hydroxy-3-(4-{(R)-2-[4-(4-hydroxy-tetrahydro-pyran-4-yl)-[1,2,3]triazol-1-ylmethyl]-pyrrolidin-1-ylmethyl}-phenyl)-acrylamide,
(E)-N-hydroxy-3-{4-[(R)-2-(4-hydroxymethyl-[1,2,3]triazol-1-ylmethyl)-pyrrolidin-1-ylmethyl]-phenyl}-acrylamide,
(E)-N-hydroxy-3-[4-((R)-2-indazol-1-ylmethyl-pyrrolidin-1-ylmethyl)-phenyl]-acrylamide,
(E)-N-hydroxy-3-[4-((R)-2-indazol-2-ylmethyl-pyrrolidin-1-ylmethyl)-phenyl]-acrylamide,
(E)-N-hydroxy-3-[4-((R)-2-pyrazol-1-ylmethyl-pyrrolidin-1-ylmethyl)-phenyl]-acrylamide,
(E)-3-{4-[(R)-2-(3,5-dimethyl-pyrazol-1-ylmethyl)-pyrrolidin-1-ylmethyl]-phenyl}-N-hydroxy-acrylamide,
(E)-3-{4-[(R)-2-(3,5-bis-trifluoromethyl-pyrazol-1-ylmethyl)-pyrrolidin-1-ylmethyl]-phenyl}-N-hydroxy-acrylamide,
(E)-3-{4-[(2R,4R)-2-(3,5-bis-trifluoromethyl-pyrazol-1-ylmethyl)-4-hydroxy-pyrrolidin-1-ylmethyl]-phenyl}-N-hydroxy-acrylamide, and
(E)-3-{4-[(2R,4R)-2-(3,5-dimethyl-pyrazol-1-ylmethyl)-4-hydroxy-pyrrolidin-1-ylmethyl]-phenyl}-N-hydroxy-acrylamide.

22. A composition comprising a therapeutically effective amount of a compound of claim 1, or a pharmaceutically acceptable salt, hydrate, or ester thereof, and a pharmaceutically acceptable carrier or excipient.

23. A method of inhibiting a deacetylase in a cell, the method comprising contacting a cell with a compound of claim 1, or a pharmaceutically acceptable salt, hydrate, or ester thereof, in an amount sufficient to inhibit a deacetylase.

24. A method of inhibiting a deacetylase in a cell, the method comprising contacting a cell with a composition of claim 22 in an amount sufficient to inhibit a deacetylase.

25. A method of treating a disease, disorder, condition, or undesired process in a mammal, the method comprising administering to a mammal a therapeutically effective amount of a compound of claim 1, or a pharmaceutically acceptable salt, hydrate, or ester thereof.

26. A method of treating a disease, disorder, condition, or undesired process in a mammal, the method comprising administering to a mammal a composition of claim 22.

27. The method of claim 25, wherein the disease, disorder, condition, or undesired process is mediated by a deacetylase.

28. The method of claim 27, wherein the deacetylase is a histone deacetylase.

29. The method of claim 25, wherein the disease, disorder, condition, or undesired process is selected from an undesired proliferative condition, a neurodegenerative disease, a cardiovascular disease, stroke, an autoimmune disease, an inflammatory disorder, an undesired immunological process, and an fungal infection.

30. The method of claim 25, wherein the disease, disorder, condition, or undesired process is selected from a cancer, a tumor, a fibrosis, a neoplasia, psoriasis, prostate hyperplasia, Alzheimer's disease, Huntington's disease, Rubenstein-Taybis syndrome, Parkinson's disease, muscular dystrophy, heart failure, cardiac hypertrophy, thrombosis, spinal muscular atrophy, stroke, Rett's syndrome, Lupus, scleroderma, atherosclerosis, and an arthritis or arthritic condition.

31. The method of claim 30, wherein the cancer is selected from brain cancer, kidney cancer, liver cancer, adrenal gland cancer, bladder cancer, breast tumor, stomach cancer, esophagus cancer, ovarian cancer, colon cancer, rectum cancer, prostate cancer, pancrea cancer, lung cancer, vagina cancer, thyroid cancer, sarcoma, glioblastomas, multiple myeloma, gastrointestinal cancer, lung cancer, colon cancer, breast cancer, ovarian cancer, bladder cancer, and leukemia.

32. The method of claim 25, wherein the mammal is a human.
